(19) United States
(12) Patent Application Publication
(10) Pub. No.: US 2021/0387983 A1
(43) Pub. Date: Dec. 16, 2021

(54) CRYSTALLINE ALK5 INHIBITORS AND USES THEREOF

(71) Applicant: THERAVANCE BIOPHARMA R&D IP, LLC, SOUTH SAN FRANCISCO, CA (US)

(72) Inventors: Alec Brozell, San Francisco, CA (US); Marta Dabros, Foster City, CA (US); Jing Fu, Foster City, CA (US); Miroslav Rapta, San Carlos, CA (US)

(73) Assignee: THERAVANCE BIOPHARMA R&D IP, LLC, SOUTH SAN FRANCISCO, CA (US)

(21) Appl. No.: 17/303,845

(22) Filed: Jun. 9, 2021

Related U.S. Application Data
(60) Provisional application No. 63/202,236, filed on Jun. 2, 2021.
(60) Provisional application No. 63/037,144, filed on Jun. 10, 2020.

(51) Int. Cl. C07D 471/04; A61K 45/06; A61P 35/04

(52) U.S. Cl. CPC C07D 471/04; A61K 45/06; A61K 9/008; C07B 2200/13; A61P 35/04

(57) ABSTRACT

The present disclosure provides crystalline forms of activin receptor-like kinase 5 (ALK5) inhibitors. Also disclosed are pharmaceutical compositions comprising the crystalline forms, methods of using the crystalline forms to modulate the activity of ALK5 and methods of treating disorders mediated by ALK5 using the crystalline forms.

### CROSS-REFERENCE

This application claims the benefit of U.S. Provisional Application No. 63/037,144, filed Jun. 10, 2020; and U.S. Provisional Application No. 63/202,236, filed Jun. 2, 2021, each of which is incorporated herein by reference in its entirety.

### BACKGROUND

Human fibrotic diseases such as systemic sclerosis (SSc), sclerodermatous graft vs. host disease, nephrogenic system fibrosis, and radiation-induced fibrosis, as well as cardiac, pulmonary, skin, liver, bladder and kidney fibrosis, constitute a major health problem. These diseases often progress to organ dysfunction with eventual organ failure and death due to lack of treatment available, mainly because the etiologic mechanisms of runaway fibrosis are complex, heterogeneous, and difficult to decipher. Activated myofibroblasts may be responsible for replacing normal tissues with nonfunctional fibrotic tissue. Therefore, signaling pathways responsible for stimulating profibrotic reactions in myofibroblasts have potential as targets for development of therapies to treat fibrotic diseases.

Normal tissue repair involves fibrotic reactions through homeostatic regulatory mechanisms. Uncontrolled fibrosis, however, may result in excess deposition of the extracellular matrix (ECM) macromolecules in interstitial space that stiffens over time. There are many sites along the molecular pathway leading up to myofibroblast activation, including, but not limited to, transforming growth factor-β (TGF-β) and bone morphogenic protein (BMP) signaling pathways. Of importance in this disclosure is the pathway involving transforming growth factor-β (TGF-β), TGF-β receptor I (TGF-βRI), and TGF-β receptor II (TGF-βRII).

TGF-β signaling is typically initiated by binding of a TGF-β ligand to a TGF-βRII. This in turn may recruit and phosphorylate TGF-βRI, also known as the activin receptor-like kinase 5 (ALK5). Once phosphorylated, ALK5 typically adopts an active conformation and is free to associate with and phosphorylate Smad2 or Smad3. Once phosphorylated, Smad 2 and 3 proteins then may form heterodimeric complexes with Smad4 which can translocate across the nuclear membrane and modulate Smad-mediated gene expression, including, for example, the production of collagen. Smad proteins are intracellular regulators of transcription and therefore may serve as modulators of TGF-β-regulated genes involving, inter alia, cell cycle arrest in epithelial and hematopoietic cells, control of mesenchymal cell proliferation and differentiation, wound healing, extracellular matrix production, immunosuppression and carcinogenesis.

ALK5 is believed to be the most relevant of the activin-like kinases (ALKs) in the fibrotic process (Rosenbloom, et al., Fibrosis: Methods and Protocols, Methods in Molecular Biology, 2017, Vol. 1627, Chapter 1, pp. 1-21). Several small molecules have been developed to inhibit the activity of ALK5 for various therapeutic indications, related mostly to oncology (see Yingling, et al., Nature Reviews: Drug Discovery, December 2004, Vol. 3, pp. 1011-1022).

### SUMMARY OF THE INVENTION

One of the main problems with ALK5 inhibitors developed to date is that these molecules have been associated with ventricular or cardiac remodeling in preclinical safety studies resulting from significant systemic exposure from oral administration. In view of the foregoing, a need exists for small molecules that target ALK5 and for use of such compounds in the treatment of various diseases, such as cancer and fibrosis, while limiting adverse side effects.

While such compounds are often initially evaluated for their activity when dissolved in solution, solid state characteristics such as polymorphism are also important. Polymorphic forms of a drug substance, such as an ALK5 inhibitor, can have different physical properties, including melting point, apparent solubility, dissolution rate, optical and mechanical properties, vapor pressure, and density. These properties can have a direct effect on the ability to process or manufacture a drug substance and the drug product. Moreover, differences in these properties can lead to different pharmacokinetics profiles for different polymorphic forms of a drug. Therefore, polymorphism is often an important factor under regulatory review of the ‘sameness’ of drug products from various manufacturers. Polymorphism can affect the quality, safety, and/or efficacy of a drug product, such as an ALK5 inhibitor. Thus, there remains a need for polymorphs of ALK5 inhibitors.

The present disclosure addresses this need and provides related advantages as well. One objective of the present disclosure is to deliver a potent ALK5 inhibitor locally with minimal systemic exposure to address any unintended and unwanted systemic side effects of ALK5 inhibition during treatment. Therefore, in some aspects, the present disclosure provides inhaled, long-acting and lung-selective ALK5 inhibitors for the treatment of idiopathic pulmonary fibrosis. Compounds, crystalline forms and salts of the present disclosure may be used to treat other diseases, including, but not limited to, pulmonary fibrosis, liver fibrosis, renal glomerulosclerosis, and cancer. Compounds of the present disclosure may be used as a monotherapy or co-dosed with other therapies, whether delivered by inhalation, orally, intravenously, subcutaneously, or topically.

In certain aspects, the present disclosure provides a crystalline form of a compound of Formula I.

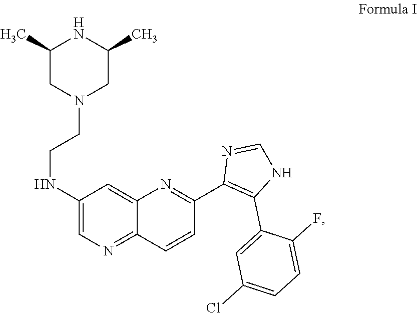 [SMILES: C[C@@H]1CN(CCNc2cnc3ccc(-c4nc[nH]c4-c4cc(Cl)ccc4F)nc3c2)C[C@H](C)N1]

or a pharmaceutically acceptable salt or solvate thereof. In some embodiments, the compound of Formula I is a fumarate salt. In some embodiments, the compound of Formula I is a mono-fumarate salt. In some embodiments, the compound of Formula I is a freebase.

The crystalline form may be polymorph Form I of a fumarate salt of the compound of Formula I. In some embodiments, the crystalline form is characterized by an X-ray powder diffraction pattern comprising peaks at 13.2±0.2, 14.9±0.2 and 22.8±0.2 degrees 2θ. The X-ray powder diffraction pattern may further comprise at least one peak, at least two peaks, or three peaks selected from 6.5±0.2, 8.9±0.2 and 17.5±0.2 degrees 2θ. In some embodiments, the X-ray powder diffraction pattern further comprises at least one peak, at least two peaks, at least three peaks, or four peaks selected from 9.5±0.2, 10.1±0.2, 18.5±0.2 and 19.5±0.2 degrees 2θ. In some embodiments, the crystalline form is characterized by an X-ray powder diffraction pattern comprising peaks at 8.9±0.2, 9.5±0.2 and 10.1±0.2 degrees 2θ. The X-ray powder diffraction pattern may further comprise at least one peak, at least two peaks, or three peaks selected from 6.5±0.2, 13.2±0.2 and 17.5±0.2 degrees 2θ. In some embodiments, the crystalline form is characterized by an X-ray powder diffraction pattern in which the peak positions are substantially in accordance with the peak positions of the pattern shown in FIG. 1.

In some embodiments, the crystalline form is characterized by a differential scanning calorimetry thermogram recorded at a heating rate of 10° C. per minute comprising an endotherm at a temperature between 260° C. and 266° C. In some embodiments, the crystalline form is characterized by a differential scanning calorimetry thermogram recorded at a heating rate of 10° C. per minute which shows a maximum in endothermic heat flow at a temperature of about 263.0±3° C. In some embodiments, the crystalline form is characterized by a differential scanning calorimetry thermogram substantially in accordance with that shown in FIG. 2. The crystalline form may be characterized by a microcrystal electron diffraction having a P2₁/n space group. In some embodiments, a single crystal comprises the unit cell dimensions: a=7.89±0.10 Å; b=18.28±0.10 Å; c=19.96±0.10 Å; α=90±0.1°; β=94.3±0.1°; and γ=90±0.1°.

The crystalline form may be polymorph Form II of a fumarate salt of the compound of Formula I. In some embodiments, the crystalline form is characterized by an X-ray powder diffraction pattern comprising peaks at 5.6±0.2, 11.2±0.2 and 15.5±0.2 degrees 2θ. The X-ray powder diffraction pattern may further comprise at least one peak, at least two peaks, or three peaks selected from 18.8±0.2, 20.6±0.2 and 22.9±0.2 degrees 2θ. In some embodiments, the X-ray powder diffraction pattern further comprises at least one peak, at least two peaks, or three peaks selected from 15.1±0.2, 22.1±0.2 and 24.8±0.2 degrees 2θ. In some embodiments, the crystalline form is characterized by an X-ray powder diffraction pattern comprising peaks at 11.2±0.2, 15.1±0.2 and 24.8±0.2 degrees 2θ. The X-ray powder diffraction pattern may further comprise at least one peak, at least two peaks, or three peaks selected from 5.6±0.2, 18.8±0.2 and 22.1±0.2 degrees 2θ. In some embodiments, the crystalline form is characterized by an X-ray powder diffraction pattern in which the peak positions are substantially in accordance with the peak positions of the pattern shown in FIG. 5.

In some embodiments, the crystalline form is characterized by a differential scanning calorimetry thermogram recorded at a heating rate of 10° C. per minute comprising an endotherm at a temperature between 259° C. and 267° C. In some embodiments, the crystalline form is characterized by a differential scanning calorimetry thermogram recorded at a heating rate of 10° C. per minute which shows a maximum in endothermic heat flow at a temperature of about 263.2±3° C. In some embodiments, the crystalline form is characterized by a differential scanning calorimetry thermogram substantially in accordance with that shown in FIG. 6. The crystalline form may be characterized by a single crystal X-ray diffraction having a P-1 space group. In some embodiments, a single crystal comprises the unit cell dimensions: a=9.42±0.10 Å; b=10.07±0.10 Å; c=16.34±0.10 Å; α=75.5±0.1°; β=87.3±0.1°; and γ=73.8±0.1°.

The crystalline form may be polymorph Form III of the compound of Formula I. In some embodiments, the crystalline form is characterized by an X-ray powder diffraction pattern comprising peaks at 10.5±0.2, 15.8±0.2 and 25.2±0.2 degrees 2θ. The X-ray powder diffraction pattern may further comprise at least one peak, at least two peaks, or three peaks selected from 7.5±0.2, 19.9±0.2 and 20.9±0.2 degrees 2θ. In some embodiments, the X-ray powder diffraction pattern further comprises at least one peak, at least two peaks, at least three peaks, or four peaks selected from 9.0±0.2, 13.2±0.2, 16.8±0.2 and 25.8±0.2 degrees 2θ. In some embodiments, the crystalline form is characterized by an X-ray powder diffraction pattern comprising peaks at 19.9±0.2, 20.9±0.2 and 25.2±0.2 degrees 2θ. The X-ray powder diffraction pattern may further comprise at least one peak, at least two peaks, or three peaks selected from 13.2±0.2, 15.8±0.2 and 25.8±0.2 degrees 2θ. In some embodiments, the crystalline form is characterized by an X-ray powder diffraction pattern in which the peak positions are substantially in accordance with the peak positions of the pattern shown in FIG. 9.

In some embodiments, the crystalline form is characterized by a differential scanning calorimetry thermogram recorded at a heating rate of 10° C. per minute comprising an endotherm at a temperature between 221° C. and 229° C. In some embodiments, the crystalline form is characterized by a differential scanning calorimetry thermogram recorded at a heating rate of 10° C. per minute which shows a maximum in endothermic heat flow at a temperature of about 224.8±3° C. In some embodiments, the crystalline form is characterized by a differential scanning calorimetry thermogram substantially in accordance with that shown in FIG. 10.

In certain aspects, the present disclosure provides a composition comprising a crystalline form of a compound of Formula I, or a pharmaceutically acceptable salt thereof. In some embodiments, the present disclosure provides a composition comprising a crystalline form of a fumarate salt of a compound of Formula I. In some embodiments, greater than about 90%, 95% or 99% by weight of the fumarate salt of the compound of Formula I in the composition is polymorph Form I. In some embodiments, greater than about 90%, 95% or 99% by weight of the fumarate salt of the compound of Formula I in the composition is polymorph Form II. In some embodiments, greater than about 90%, 95% or 99% by weight of the compound of Formula I in the composition is polymorph Form III. In some embodiments, greater than about 90%, 95% or 99% by weight of the compound of Formula I in the composition is a single conformational polymorph. In some embodiments, the composition can be stored at about 40° C. and 75% relative humidity for at least 30 days without significant degradation or change in the crystalline form. In some embodiments, the composition can be stored at about 60° C. and 75% relative humidity for at least 30 days without significant degradation or change in the crystalline form. The crystalline form may be anhydrous, slightly-hygroscopic or both.

In certain aspects, the present disclosure provides a fumarate salt of a compound of Formula I.

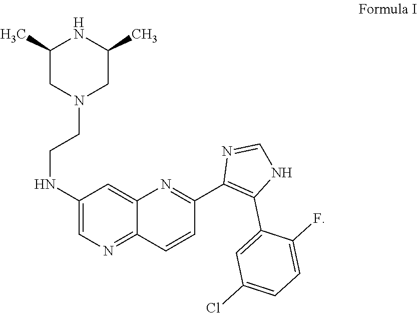 [SMILES: C[C@@H]1CN(CCNc2cnc3ccc(-c4nc[nH]c4-c4cc(Cl)ccc4F)nc3c2)C[C@H](C)N1]

In certain aspects, the present disclosure provides a fumarate salt which is 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric acid. In some embodiments, the compound of Formula I is a mono-fumarate salt. In certain aspects, the present disclosure provides a crystalline form of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric acid, prepared from 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, trihydrochloride.

In certain aspects, the present disclosure provides a pharmaceutical composition comprising a crystalline form, a composition or a fumarate salt disclosed herein, and a pharmaceutically acceptable carrier. In some embodiments, the pharmaceutical composition is formulated for inhalation.

In certain aspects, the present disclosure provides a method of inhibiting ALK5, comprising contacting ALK5 with an effective amount of a crystalline form, a composition or a fumarate salt disclosed herein. In certain aspects, the present disclosure provides a method of treating an ALK5-mediated disease or condition in a subject, comprising administering to the subject a therapeutically effective amount of a crystalline form, a composition or a fumarate salt disclosed herein. The disease or condition may be selected from fibrosis, alopecia and cancer, such as fibrosis. In certain aspects, the present disclosure provides a method of treating fibrosis, comprising administering to a patient a therapeutically effective amount of a crystalline form, a composition or a fumarate salt disclosed herein. The fibrosis may be selected from systemic sclerosis, nephrogenic systemic fibrosis, organ-specific fibrosis, fibrosis associated with cancer, cystic fibrosis, and fibrosis associated with an autoimmune disease. In some embodiments, the organ-specific fibrosis is selected from cardiac fibrosis, kidney fibrosis, pulmonary fibrosis, liver fibrosis, portal vein fibrosis, skin fibrosis, bladder fibrosis, intestinal fibrosis, peritoneal fibrosis, myelofibrosis, oral submucous fibrosis, and retinal fibrosis, such as intestinal fibrosis. In some embodiments, the pulmonary fibrosis is selected from idiopathic pulmonary fibrosis (IPF), familial pulmonary fibrosis (FPF), interstitial lung fibrosis, fibrosis associated with asthma, fibrosis associated with chronic obstructive pulmonary disease (COPD), silica-induced fibrosis, asbestos-induced fibrosis and chemotherapy-induced lung fibrosis, such as idiopathic pulmonary fibrosis (IPF). In some embodiments, the pulmonary fibrosis was induced by a viral infection. In some embodiments, the disease or condition is cancer, optionally wherein the cancer is selected from breast cancer, colon cancer, prostate cancer, lung cancer, hepatocellular carcinoma, glioblastoma, melanoma, and pancreatic cancer. The lung cancer may be non-small cell lung cancer.

In practicing any of the subject methods, the method may further comprise administering a second therapeutic agent, optionally wherein the second therapeutic agent is an immunotherapeutic agent, such as a PD-1 inhibitor or a CTLA-4 inhibitor. In some embodiments, the immunotherapeutic agent is selected from pembrolizumab and durvalumab. A method disclosed herein may further comprise administering an effective amount of radiation. In practicing any of the subject methods, the crystalline form, composition or fumarate salt may be administered by inhalation.

In certain aspects, the present disclosure provides a method of preparing a compound of Formula I, the method comprising:

(a) coupling a compound of Formula 1a:

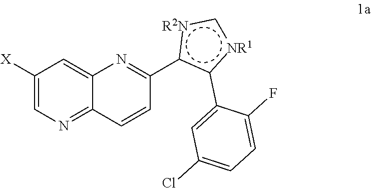

with a compound of Formula 1b:

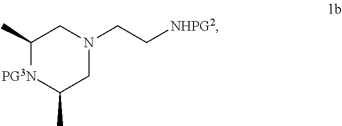

to provide a compound of Formula 1c:

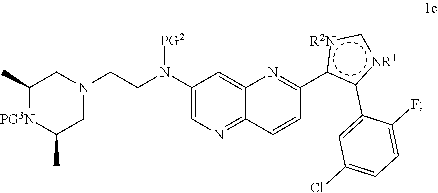

and

(b) deprotecting the compound of Formula 1c to provide the compound of Formula I, or a tautomer thereof, wherein: R¹ is PG¹ and R² is absent, or R¹ is absent and R² is PG¹; and PG¹, PG² and PG³ are each independently hydrogen or a protecting group, wherein at least one of PG¹, PG² and PG³ is a protecting group. PG¹, PG² and PG³ may each independently be hydrogen or a protecting group selected from carbobenzyloxy (Cbz), p-methoxybenzyl carbonyl (Moz), tert-butyloxycarbonyl (Boc), 9-fluorenylmethyloxycarbonyl (Fmoc), acetyl (Ac), benzoyl (Bz), benzyl (Bn), p-methyoxybenzyl (PMB), 3,4-dimethoxybenzyl (DMPM), p-methoxyphenyl (PMP), tosyl (Ts), tetrahydropyran (THP), trichloroethylchloroformate (Troc), and trimethylsilylethoxymethyl (SEM). In some embodiments, PG¹, PG² and PG³ are each independently selected from Boc and SEM. The coupling may comprise a palladium catalyst. In some embodiments, the compound of Formula 1a is 7-bromo-2-(5-(5-chloro-2-fluorophenyl)-1-((2-(trimethylsilyl)ethoxy)methyl)-1H-imidazol-4-yl)-1,5-naphthyridine or 7-bromo-2-(4-(5-chloro-2-fluorophenyl)-1-((2-(trimethylsilyl)ethoxy)methyl)-1H-imidazol-5-yl)-1,5-naphthyridine. In some embodiments, the compound of Formula 1b is tert-butyl (2S,6R)-4-(2-((tert-butoxycarbonyl)amino)ethyl)-2,6-dimethylpiperazine-1-carboxylate. In some embodiments, the compound of Formula 1c is tert-butyl (2S,6R)-4-(2-((tert-butoxycarbonyl)(6-(4-(5-chloro-2-fluorophenyl)-1-((2-(trimethylsilyl)ethoxy)methyl)-1H-imidazol-5-yl)-1,5-naphthyridin-3-yl)amino)ethyl)-2,6-dimethylpiperazine-1-carboxylate or tert-butyl (2S,6R)-4-(2-((tert-butoxycarbonyl)(6-(5-(5-chloro-2-fluorophenyl)-1-((2-(trimethylsilyl)ethoxy)methyl)-1H-imidazol-4-yl)-1,5-naphthyridin-3-yl)amino)ethyl)-2,6-dimethylpiperazine-1-carboxylate.

In certain aspects, the present disclosure provides a method of preparing a crystalline form disclosed herein, the method comprising: (a) combining the compound of Formula I, solvent and fumaric acid, thereby forming a mixture; (b) stirring the mixture; and (c) isolating the crystalline fumarate salt from the mixture. The mixture may be heated, optionally to about 80° C. In some embodiments, the solvent is selected from acetone, acetonitrile, ethyl acetate, methyl ethyl ketone, methanol, ethanol, 2-propanol, isobutanol, t-butanol, dichloromethane, 1,4-dioxane, isopropyl acetate, toluene, methyl t-butyl ether, cyclopentyl methyl ether, hexanes, tetrahydrofuran, water, and combinations thereof. In some embodiments, the solvent is selected from 2-propanol, water, and combinations thereof. In some embodiments, the method further comprises, prior to (a): (a-1) dissolving a trihydrochloride salt of the compound of Formula I in water, thereby forming a salt solution; (a-2) adding to the salt solution a mixture of base and solvent, thereby forming a biphasic mixture comprising an aqueous phase and an organic phase, wherein the organic phase comprises the compound of Formula I; and (a-3) removing the aqueous phase from the biphasic mixture. In some embodiments, the present disclosure provides a crystalline form of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric acid prepared according to a method described herein.

In certain aspects, the present disclosure provides a crystalline form of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric acid, prepared from 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, trihydrochloride. In certain aspects, the present disclosure provides a crystalline form of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric acid, prepared according to a method disclosed herein. In certain aspects, the present disclosure provides a crystalline form of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, freebase, prepared from 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, trihydrochloride. In certain aspects, the present disclosure provides a crystalline form of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, freebase, prepared according to a method disclosed herein.

### INCORPORATION BY REFERENCE

All publications and patent applications mentioned in this specification are herein incorporated by reference to the same extent as if each individual publication or patent application was specifically and individually indicated to be incorporated by reference.

### DESCRIPTION OF THE DRAWINGS

The novel features of the disclosure are set forth with particularity in the appended claims. An understanding of the features and advantages of the present disclosure may be obtained by reference to the following detailed description that sets forth illustrative embodiments, in which the principles of the disclosure are utilized, and the accompanying drawings, of which:

FIG. 1 shows an X-ray powder diffraction (XRPD) pattern of polymorph Form I of a fumarate salt of the compound of Formula I.

FIG. 2 shows an exemplary differential scanning calorimetry (DSC) thermogram of polymorph Form I of a fumarate salt of the compound of Formula I.

FIG. 3 shows a thermal gravimetric analysis (TGA) plot of polymorph Form I of a fumarate salt of the compound of Formula I.

FIG. 4 shows a dynamic moisture sorption (DMS) isotherm of polymorph Form I of a fumarate salt of the compound of Formula I, observed at a temperature of about 25° C.

FIG. 5 shows an X-ray powder diffraction (XRPD) pattern of polymorph Form II of a fumarate salt of the compound of Formula I.

FIG. 6 shows an exemplary differential scanning calorimetry (DSC) thermogram of polymorph Form II of a fumarate salt of the compound of Formula I.

FIG. 7 shows a thermal gravimetric analysis (TGA) plot of polymorph Form II of a fumarate salt of the compound of Formula I.

FIG. 8 shows a dynamic moisture sorption (DMS) isotherm of polymorph Form II of a fumarate salt of the compound of Formula I, observed at a temperature of about 25° C.

FIG. 9 shows an X-ray powder diffraction (XRPD) pattern of polymorph Form III of the compound of Formula I (freebase).

FIG. 10 shows an exemplary differential scanning calorimetry (DSC) thermogram of polymorph Form III of the compound of Formula I (freebase).

FIG. 11 shows a thermal gravimetric analysis (TGA) plot of polymorph Form III of the compound of Formula I (freebase).

FIG. 12 shows a dynamic moisture sorption (DMS) isotherm of polymorph Form III of the compound of Formula I (freebase), observed at a temperature of about 25° C.

### DETAILED DESCRIPTION

Unless defined otherwise, all technical and scientific terms used herein have the same meaning as is commonly understood by one of skill in the art to which this disclosure belongs.

Chemical structures are named herein according to IUPAC conventions as implemented in ChemDraw® software (Perkin Elmer, Inc., Cambridge, Mass.).

As used in the specification and claims, the singular forms “a”, “an” and “the” include plural references unless the context clearly dictates otherwise.

The term “pharmaceutically acceptable” refers to a material that is not biologically or otherwise unacceptable when used in the subject compositions and methods. For example, the term “pharmaceutically acceptable carrier” refers to a material—such as an adjuvant, excipient, glidant, sweetening agent, diluent, preservative, dye, colorant, flavor enhancer, surfactant, wetting agent, dispersing agent, suspending agent, stabilizer, isotonic agent, solvent or emulsifier—that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The terms “salt” and “pharmaceutically acceptable salt” refer to a salt prepared from a base or an acid. Pharmaceutically acceptable salts are suitable for administration to a patient, such as a mammal (for example, salts having acceptable mammalian safety for a given dosage regime). Salts can be formed from inorganic bases, organic bases, inorganic acids and organic acids. In addition, when a compound contains both a basic moiety, such as an amine, pyridine or imidazole, and an acidic moiety, such as a carboxylic acid or tetrazole, zwitterions may be formed and are included within the term “salt” as used herein. Salts derived from inorganic bases include ammonium, calcium, copper, ferric, ferrous, lithium, magnesium, manganic, manganous, potassium, sodium, and zinc salts, and the like. Salts derived from organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Salts derived from inorganic acids include salts of boric, carbonic, hydrohalic (hydrobromic, hydrochloric, hydrofluoric or hydroiodic), nitric, phosphoric, sulfamic and sulfuric acids. Salts derived from organic acids include salts of aliphatic hydroxyl acids (for example, citric, gluconic, glycolic, lactic, lactobionic, malic, and tartaric acids), aliphatic monocarboxylic acids (for example, acetic, butyric, formic, propionic and trifluoroacetic acids), amino acids (for example, aspartic and glutamic acids), aromatic carboxylic acids (for example, benzoic, p-chlorobenzoic, diphenylacetic, gentisic, hippuric, and triphenylacetic acids), aromatic hydroxyl acids (for example, o-hydroxybenzoic, p-hydroxybenzoic, 1-hydroxynaphthalene-2-carboxylic, and 3-hydroxynaphthalene-2-carboxylic acids), ascorbic, dicarboxylic acids (for example, fumaric, maleic, oxalic and succinic acids), glucoronic, mandelic, mucic, nicotinic, orotic, pamoic, pantothenic, sulfonic acids (for example, benzenesulfonic, camphorsulfonic, edisylic, ethanesulfonic, 1,2-ethanedisulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the present disclosure provides a fumarate salt, a 1,2-ethanedisulfonate salt, or a 1-hydroxy-2-naphthoate salt. In some embodiments, the present disclosure provides a fumarate salt, such as a mono-fumarate salt.

The term “therapeutically effective amount” refers to that amount of a compound described herein that is sufficient to affect treatment when administered to a subject in need thereof. For example, a therapeutically effective amount for treating pulmonary fibrosis is an amount of compound needed to, for example, reduce, suppress, eliminate, or prevent the formation of fibrosis in a subject, or to treat the underlying cause of pulmonary fibrosis. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The specific dose will vary depending on the particular compound chosen, the dosing regimen to be followed, whether it is administered in combination with other compounds, timing of administration, the tissue to which it is administered, and the physical delivery system in which it is carried. The term “effective amount” refers to an amount sufficient to obtain a desired result, which may not necessarily be a therapeutic result. For example, an “effective amount” may be the amount needed to inhibit an enzyme.

As used herein, “treating” or “treatment” refers to an approach for obtaining beneficial or desired results with respect to a disease, disorder, or medical condition (such as pulmonary fibrosis) in a subject, including but not limited to the following: (a) preventing the disease or medical condition from occurring, e.g., preventing the reoccurrence of the disease or medical condition or prophylactic treatment of a subject that is pre-disposed to the disease or medical condition; (b) ameliorating the disease or medical condition, e.g., eliminating or causing regression of the disease or medical condition in a subject; (c) suppressing the disease or medical condition, e.g., slowing or arresting the development of the disease or medical condition in a subject; or (d) alleviating symptoms of the disease or medical condition in a subject. For example, “treating pulmonary fibrosis” would include preventing fibrosis from occurring, ameliorating fibrosis, suppressing fibrosis, and alleviating the symptoms of fibrosis (for example, increasing oxygen levels in blood or improved lung function tests). Also, a therapeutic benefit is achieved with the eradication or amelioration of one or more of the physiological symptoms associated with the underlying disorder such that an improvement is observed in the subject, notwithstanding that the subject may still be afflicted with the underlying disorder

A “therapeutic effect”, as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “antagonist” and “inhibitor” are used interchangeably, and they refer to a compound having the ability to inhibit a biological function (e.g., activity, expression, binding, protein-protein interaction) of a target protein (e.g., ALK5). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition.

The term “selective inhibition” or “selectively inhibit” refers to the ability of a biologically active agent to preferentially reduce the target signaling activity as compared to off-target signaling activity, via direct or indirect interaction with the target.

As used herein, the term “antibody” refers to an immunoglobulin molecule that specifically binds to, or is immunologically reactive toward, a specific antigen. An antibody may be, for example, polyclonal, monoclonal, genetically engineered, or an antigen binding fragment thereof, and further may be, for example, murine, chimeric, humanized, heteroconjugate, bispecific, a diabody, a triabody, or a tetrabody. An antigen binding fragment includes an antigen binding domain and may be in the form of, for example, a Fab′, F(ab′)₂, Fab, Fv, rIgG, scFv, hcAbs (heavy chain antibodies), a single domain antibody, VHH, VNAR, sdAb, or nanobody.

The term “antigen binding domain” as used herein refers to a region of a molecule that binds to an antigen. An antigen binding domain may be an antigen-binding portion of an antibody or an antibody fragment. An antigen binding domain may be one or more fragments of an antibody that retain the ability to specifically bind to an antigen. An antigen binding domain can be an antigen binding fragment and may recognize a single antigen, two antigens, three antigens or more. As used herein, “recognize” with regard to antibody interactions refers to the association or binding between an antigen binding domain of an antibody or portion thereof and an antigen.

An “antibody construct” refers to a molecule, e.g., a protein, peptide, antibody or portion thereof, that contains an antigen binding domain and an Fc domain (e.g., an Fc domain from within the Fc region). An antibody construct may recognize, for example, one antigen or multiple antigens.

A “targeting moiety” refers to a structure that has a selective affinity for a target molecule relative to other non-target molecules. The targeting moiety binds to a target molecule. A targeting moiety may include an antibody, a peptide, a ligand, a receptor, or a binding portion thereof. The target biological molecule may be a biological receptor or other structure of a cell, such as a tumor antigen.

The terms “subject” and “patient” refer to an animal, such as a mammal (e.g., a human), that has been or will be the object of treatment, observation or experiment. The methods described herein can be useful in both human therapy and veterinary applications. In some embodiments, the subject is a mammal, and in some embodiments, the subject is human. “Mammal” includes humans and both domestic animals such as laboratory animals and household pets (e.g., cats, dogs, swine, cattle, sheep, goats, horses, rabbits), and non-domestic animals such as wildlife and the like.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates. Also included are solvates formed with one or more crystallization solvents.

“Crystalline form”, “polymorph”, “Form”, and “form” may be used interchangeably herein, and are meant to include all crystalline forms of a compound, including, for example, polymorphs, pseudopolymorphs, salts, solvates, hydrates, unsolvated polymorphs (including anhydrates), and conformational polymorphs of the compounds, as well as mixtures thereof, unless a particular crystalline form is referred to. Compounds of the present disclosure include crystalline forms of those compounds, including, for example, polymorphs, pseudopolymorphs, salts, solvates, hydrates, unsolvated polymorphs (including anhydrates), and conformational polymorphs of the compounds, as well as mixtures thereof.

The term “tautomer”, as used herein, refers to each of two or more isomers of a compound that exist in equilibrium and which readily interconvert. For example, one skilled in the art would understand that 1,2,3-triazole exists in two tautomeric forms:

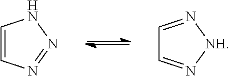 [SMILES: c1c[nH]nn1 ; c1cn[nH]n1]

Unless otherwise specified, chemical entities described herein are intended to include all possible tautomers, even when a structure depicts only one of them. For example, even though a single tautomer of a compound of Formula I may be depicted herein, the disclosure is intended to include all possible tautomers, such as:

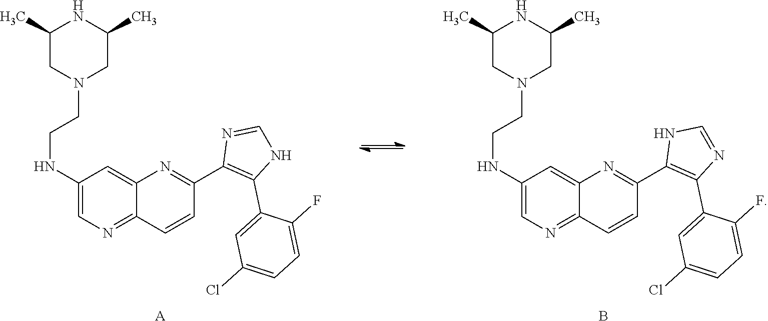

In this example, the two depicted tautomers give rise to different numbering of the imidazole ring under IUPAC naming conventions: 6-(5-(5-chloro-2-fluorophenyl)-1H-imidazol-4-yl)-N-(2-((3R,5S)-3,5-dimethylpiperazin-1-yl)ethyl)-1,5-naphthyridin-3-amine (A) and 6-(4-(5-chloro-2-fluorophenyl)-1H-imidazol-5-yl)-N-(2-((3R,5S)-3,5-dimethylpiperazin-1-yl)ethyl)-1,5-naphthyridin-3-amine (B). Even though tautomer A is typically named and depicted herein, it will be understood that the disclosure also includes tautomer B, unless otherwise specified.

Additionally, chemical entities described herein are intended to include all possible conformational isomers and/or all solid form conformational polymorphs, even when a depicted structure does not provide conformational detail or appears to depict only a single conformer. Even though a single conformer of a compound of Formula I may be depicted herein, the disclosure is intended to include all possible conformers. For example, the compound of Formula I includes both conformers A and C depicted below, wherein five contiguous bonds have been thickened to indicate co-planarity between the napthyridine ring and the imidazole ring:

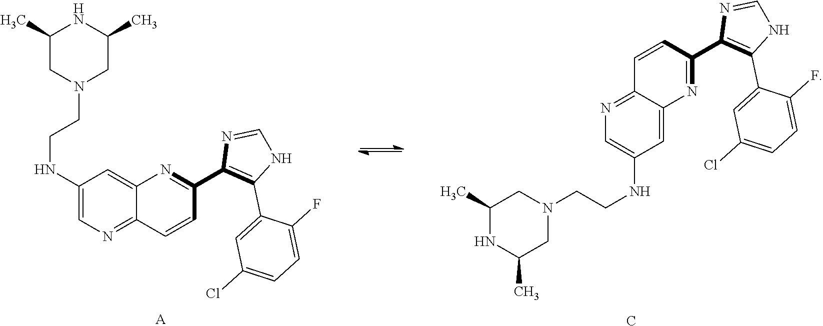

In certain aspects, the present disclosure provides a crystalline form of the compound of Formula I, or a salt thereof, as a substantially pure conformational polymorph. In some embodiments, the conformational polymorph is provided in at least 90% excess, such as at least 95%, 98% or 99% excess. A crystalline form described herein may consist of a single conformer of the compound of Formula I. In some embodiments, more than one conformer is present in the same crystalline form. In some embodiments, the present disclosure provides a conformational polymorph of Formula A. In some embodiments, the present disclosure provides a conformational polymorph of Formula C. In some embodiments, the compound of Formula I is provided as a substantially pure conformational isomer. In some embodiments, the conformational isomer is provided in at least 90% excess.

The discovery of a crystalline form of a compound of Formula I was particularly challenging due to the reduced crystallization tendency of the compound, in both freebase and fumarate salt forms. The present inventors found that the compound of Formula I and its fumarate salt exhibit an unusually wide metastable zone. Additionally, significant yield was lost to the mother liquor, despite low solubility of the crystalline product in the mother liquor. Poor impurity purging was also observed in the crystallization process, and small changes in the purity of the crystalline form were typically found to have dramatic effects on solubility.

Surprising, the present inventors found that crystallization of the freebase and fumarate salt forms is accelerated with increasing temperature, which is opposite the usual trend a skilled person would expect based on typical temperature and solubility relationships. Not wishing to be bound by any particular theory, the present inventors hypothesize that competing conformational polymorphs for both the freebase and fumarate salt forms have the effect of suppressing crystallinity of the compound of Formula I or salt thereof. While the bond rotation depicted above for conformers A and C of the compound of Formula I occurs freely in solution, it is believed to settle into one of the two conformations represented by the depictions above upon crystallization. Polymorph Form I is believed to be the kinetic polymorph conformation, roughly depicted in 2D by conformer A above, while polymorph Form II is believed to be the thermodynamic polymorph conformation, rough depicted in 2D by conformer C above.

The compound of Formula I described herein comprises two asymmetric centers and can thus give rise to diastereomers, the asymmetric centers of which can each be defined, in terms of absolute stereochemistry, as (R)- or (S)-. In some embodiments, in order to optimize the therapeutic activity of the compounds of the disclosure, e.g., to treat fibrosis, it may be desirable that the carbon atoms have a particular configuration (e.g., (R,R), (S,S), or (S,R)/(R,S)) or are enriched in a stereoisomeric form having such configuration. In some embodiments, the compound of Formula I, as shown and named, is in the (3R,5S) configuration, which is equivalent to the (3S,5R) configuration. It will be understood by those skilled in the art that minor amounts of other stereoisomers may be present in the compositions of the disclosure unless otherwise indicated, provided that the utility of the composition as a whole is not eliminated by the presence of such other isomers. In some embodiments, the compound of Formula I is provided as a substantially pure stereoisomer. In some embodiments, the stereoisomer is provided in at least 90% diastereomeric excess.

It is further understood that compounds A and D depicted below represent the same compound due to internal symmetry in the compound, even though the depicted structures may give rise to different IUPAC names (e.g., 6-(5-(5-chloro-2-fluorophenyl)-1H-imidazol-4-yl)-N-(2-((3R,5S)-3,5-dimethylpiperazin-1-yl)ethyl)-1,5-naphthyridin-3-amine (A) and 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine (D).

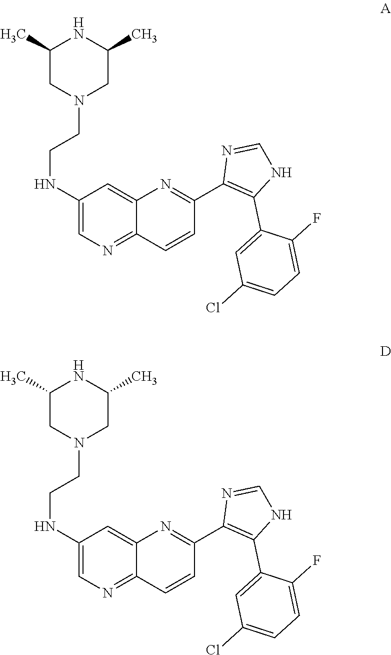 [SMILES: C[C@@H]1CN(CCNc2cnc3ccc(-c4nc[nH]c4-c4cc(Cl)ccc4F)nc3c2)C[C@H](C)N1 ; C[C@@H]1CN(CCNc2cnc3ccc(-c4nc[nH]c4-c4cc(Cl)ccc4F)nc3c2)C[C@H](C)N1]

When ranges are used herein for physical properties, such as molecular weight, or chemical properties, such as chemical formulae, all combinations and sub-combinations of ranges and specific embodiments therein are intended to be included.

The term “about” when referring to a number or a numerical range means the number or numerical range referred to is an approximation within experimental variability (or within statistical experimental error), and thus the number or numerical range may vary from, for example, between 1% and 15% of the stated number or numerical range. Optionally, the term “about” may indicate that the number or numerical range is within ±5% of the specified number or range.

The term “substantially” when referring, for example, to an X-ray powder diffraction pattern or a differential scanning calorimetry thermogram, includes a pattern or thermogram that is not necessarily identical to those depicted herein, but that falls within the limits of experimental error or deviations when considered by one of ordinary skill in the art. For example, the peaks of a first X-ray powder diffraction pattern may be considered substantially in accordance with those of a reference diffraction pattern if at least 90% of the peaks in the first diffraction pattern are positioned within ±0.5 degrees 2θ—such as ±0.4 degrees 2θ, ±0.3 degrees 2θ, or ±0.2 degrees 2θ—of the peak positions of the reference diffraction pattern.

Lung function tests include tests to check how well the lungs work. Spirometry, for example, measures the amount of air the lungs can hold as well as how forcefully one can empty air from the lungs. Forced expiratory volume (FEV) is a measure of the amount of air a person can exhale during a forced breath. FEV1, for example, is the amount of air a person can force from their lungs in one second. Forced vital capacity (FVC) is the total amount of air exhaled during an FEV test. The ratio of FEV1/FVC, also known as Index of Air Flow or Tiffeneau-Pinelli Index, is a measurement used to assess the health of a patient's lung function. A ratio of <70% indicates an obstructive defect is present in the lungs, such as chronic obstructive pulmonary disease (COPD). A ratio of >80% indicates a restrictive defect is present in the lungs, such as pulmonary fibrosis. The ratio of >80% in restrictive lung disease results from both FEV1 and FVC being reduced but that the decline in FVC is more than that of FEV1, resulting in a higher than 80% value.

The term “transforming growth factor-β” may also be referred to as TGF-β, transforming growth factor beta-1, or TGF-beta-1. It is also cleaved into latency-associated peptide (LAP).

The term “TGF-β receptor II” may also be referred to as TβRII, type II TGF-β receptor, TGF-βRII, TGF-beta receptor type-2, TGFR-2, TGF-beta type II receptor, transforming growth factor-beta receptor type II, TGF-beta receptor type II or TbetaR-II.

The term “TGF-β receptor I” may also be referred to as TORI, type I TGF-β receptor, TGF-βRI, TGF-beta receptor type-1, TGFR-1, activin A receptor type II-like protein kinase of 53 kD, activin receptor-like kinase 5, ALK-5, ALK5, serine/threonine-protein kinase receptor R⁴, SKR4, TGF-beta type I receptor, transforming growth factor-beta receptor type I, TGF-beta receptor type I, transforming growth factor beta receptor I, TGF-beta receptor 1, or TbetaR-I.

The present disclosure provides compounds that are capable of selectively binding to and/or modulating ALK5. In some embodiments, the compounds modulate ALK5 by binding to or interacting with one or more amino acids and/or one or more metal ions. The binding of these compounds may disrupt ALK5 downstream signaling.

The polymorphs disclosed herein may be characterized by any appropriate methodology according to the art. For example, a polymorph made according to the present disclosure may be characterized by X-ray powder diffraction (XRPD), differential scanning calorimetry (DSC), thermogravimetric analysis (TGA), hot-stage microscopy, and/or spectroscopy (e.g., Raman, solid state nuclear magnetic resonance (ssNMR), and infrared (IR)).

XRPD: A polymorph of the present disclosure may be characterized by XRPD. The relative intensities of XRPD peaks can vary, depending upon the particle size, the sample preparation technique, the sample mounting procedure and the particular instrument employed. Moreover, instrument variation and other factors can affect the 20 values. Therefore, the XRPD peak assignments can vary, for example, by ±0.5 degrees 2θ, such as ±0.4, ±0.3 or ±0.2 degrees 2θ.

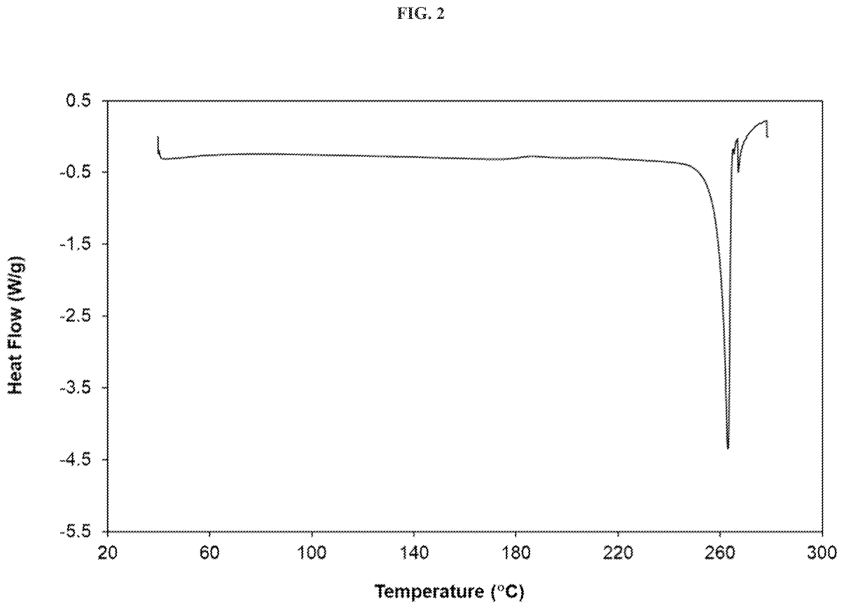

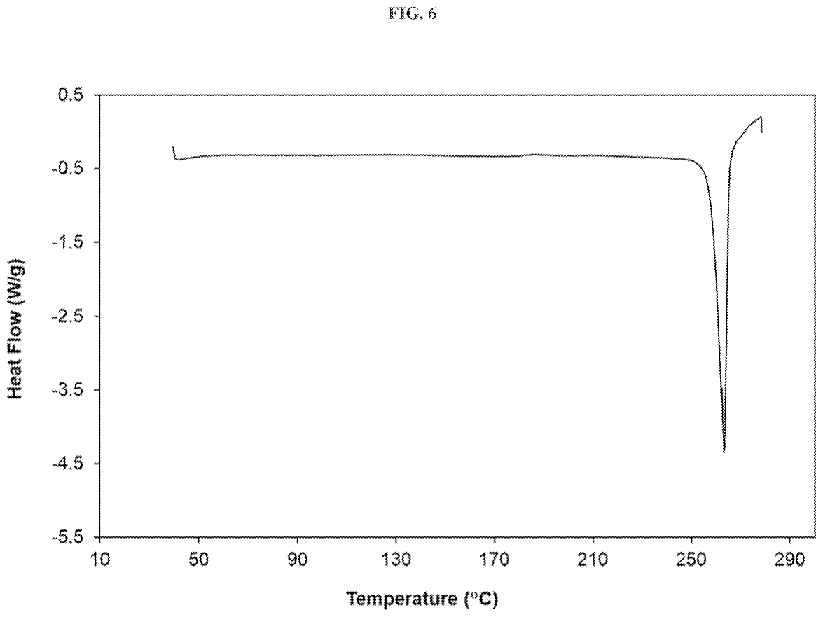

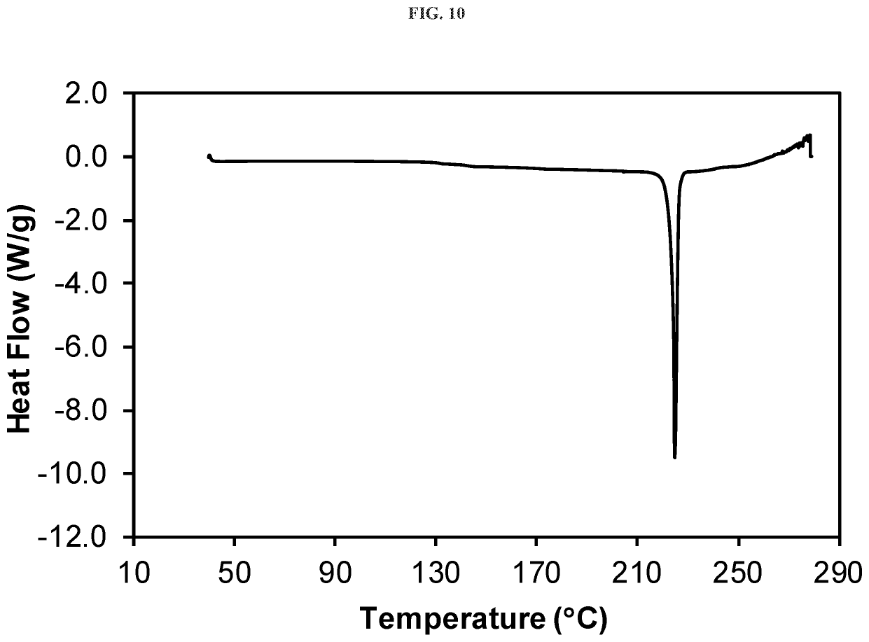

DSC: A polymorph of the present disclosure can also be identified by its characteristic DSC thermogram, such as the thermograms depicted in FIG. 2, FIG. 6 or FIG. 10. The temperatures observed in a DSC thermogram may depend upon the rate of temperature change, as well as the sample preparation technique and the particular instrument employed. Thus, the values reported herein relating to DSC thermograms can vary, for example, by ±6° C., such as ±5 or ±4° C.

TGA: A polymorph of the present disclosure may also give rise to thermal behavior different from that of the amorphous material or another solid form. Thermal behavior may be assessed in the laboratory by thermogravimetric analysis (TGA), which may be used to distinguish some polymorphic forms from others. A polymorph described herein may be characterized by thermogravimetric analysis.

General Synthetic Procedures

Polymorphs of a compound of Formula I or salt thereof can be synthesized from readily available starting materials as described below and in the Examples. Materials used herein are either commercially available or prepared by synthetic methods generally known in the art. The examples below are not limited to the compounds listed or by any particular substituents, which are employed for illustrative purposes. Although various steps are described and depicted, the steps in some cases may be performed in a different order than shown in the examples below. Various modifications to these synthetic reaction schemes may be made and will be suggested to one skilled in the art having referred to this disclosure. For example, where typical or preferred process conditions (e.g., reaction temperatures, crystallization temperatures, reaction times, mole ratios of reactants, solvents, pressures, etc.) are given, other process conditions may also be used unless otherwise stated. In some instances, reactions or crystallizations were conducted at room temperature and no actual temperature measurement was taken. It is understood that room temperature means a temperature within the range commonly associated with the ambient temperature in a laboratory environment, and will typically be in the range of about 15° C. to about 30° C., such as about 20° C. to about 25° C.

In general, the compound of Formula I may be prepared according to the following reaction scheme:

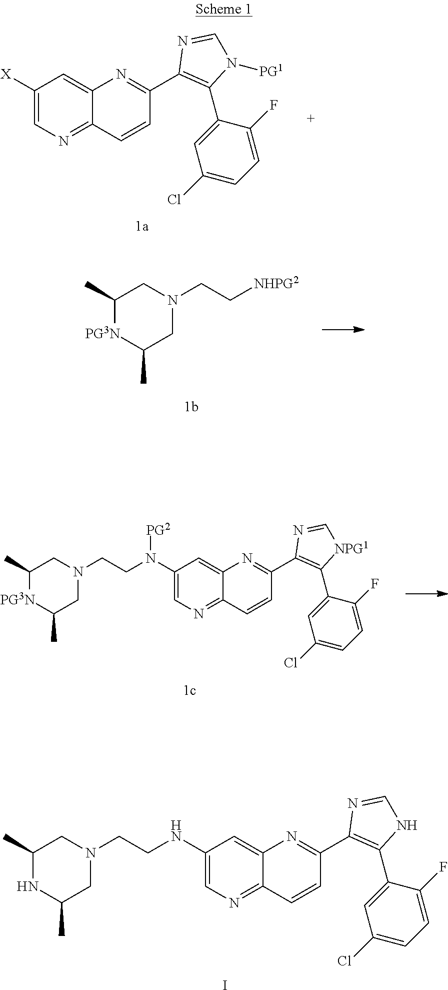

In some embodiments, the compound of Formula I may be prepared according to Scheme 1. For example, heteroaryl halide 1a can be subjected to a C—N coupling reaction—optionally a Pd-catalyzed coupling reaction such as a Buchwald-Hartwig amination—with protected amine 1b to provide a heteroaryl amine of Formula 1c. Deprotection of 1c may reveal the compound of Formula I.

In certain aspects, the present disclosure provides a method of preparing a compound of Formula I, the method comprising forming a C—N bond, optionally via a Buchwald-Hartwig amination. In some embodiments, the method further comprises removing one or more protecting groups, such as an amino protecting group.

In certain aspects, the present disclosure provides a method of preparing a compound of Formula I, the method comprising:

(a) coupling a compound of Formula 1a:

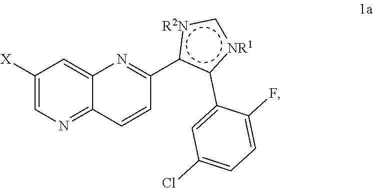

with a compound of Formula 1b:

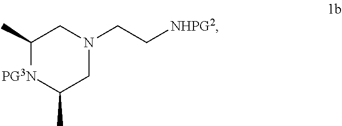

to provide a compound of Formula 1c:

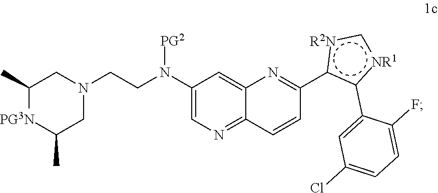

and

(b) deprotecting the compound of Formula 1c to provide the compound of Formula I:

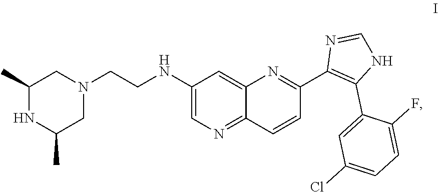 [SMILES: C[C@@H]1CN(CCNc2cnc3ccc(-c4nc[nH]c4-c4cc(Cl)ccc4F)nc3c2)C[C@H](C)N1]

or a tautomer thereof,
wherein: R¹ is PG¹ and R² is absent, or RI is absent and R² is PG¹; and PG¹, PG² and PG³ are each independently hydrogen or a protecting group. In some embodiments, PG¹, PG² and PG³, each together with the nitrogen atom to which it is attached, independently form a carbamate, an acetamide, a phthalimide, a benzylamine, a tritylamine, a benzylideneamine, or a sulfonamide. In some embodiments, PG¹, PG² and PG³ are each independently hydrogen or a protecting group selected from carbobenzyloxy (Cbz), p-methoxybenzyl carbonyl (Moz), tert-butyloxycarbonyl (Boc), 9-fluorenylmethyloxycarbonyl (Fmoc), acetyl (Ac), benzoyl (Bz), benzyl (Bn), p-methyoxybenzyl (PMB), 3,4-dimethoxybenzyl (DMPM), p-methoxyphenyl (PMP), tosyl (Ts), tetrahydropyran (THP), trichloroethylchloroformate (Troc), and trimethylsilylethoxymethyl (SEM). In some embodiments, PG¹, PG² and PG³ are each independently selected from Boc and SEM. In some embodiments, PG¹, PG² and PG³ are each independently selected from Boc, THP and SEM. In some embodiments, PG¹ is SEM. In some embodiments, PG¹ is THP. In some embodiments, PG² and PG³ are each Boc. In some embodiments, PG¹ is SEM and PG² and PG³ are each Boc. In some embodiments, PG¹ is hydrogen. In some embodiments, PG² is hydrogen. In some embodiments, PG³ is hydrogen. In some embodiments, PG¹ and PG² are each hydrogen, and PG³ is a protecting group, such as Boc, THP or SEM. In some embodiments, PG¹ is hydrogen and PG² and PG³ are each independently a protecting group, such as Boc, THP or SEM. In some embodiments, PG¹ and PG² are each independently hydrogen or a protecting group, and PG³ is a protecting group. In some embodiments, the molar ratio of 1a to 1b is between 1:1 and 1:1.5, such as 1:1.2.

In some embodiments, the coupling comprises a palladium catalyst, such as Pd₂dba₃. In some embodiments, the coupling comprises a ligand. The ligand may be a phosphine ligand, optionally a bidentate phosphine ligand, such as XantPhos. In some embodiments, the coupling comprises a base, such as t-BuONa. In some embodiments, the coupling comprises a palladium catalyst, a ligand and a base. In some embodiments, the coupling comprises Pd₂dba₃, XantPhos and t-BuONa. In some embodiments, the coupling is a Buchwald-Hartwig amination. The coupling may comprise a solvent, such as toluene.

In some embodiments, the deprotecting comprises an acid, such as TFA or HCl. If a salt is formed in the deprotecting, the process may further comprise freebasing the salt, for example, by addition of a base, such as NaOH or NH₄OH.

In some embodiments, the compound of Formula 1a is 7-bromo-2-(5-(5-chloro-2-fluorophenyl)-1-((2-(trimethylsilyl)ethoxy)methyl)-1H-imidazol-4-yl)-1,5-naphthyridine or 7-bromo-2-(4-(5-chloro-2-fluorophenyl)-1-((2-(trimethylsilyl)ethoxy)methyl)-1H-imidazol-5-yl)-1,5-naphthyridine. In some embodiments, the compound of Formula 1b is tert-butyl (2S,6R)-4-(2-((tert-butoxycarbonyl)amino)ethyl)-2,6-dimethylpiperazine-1-carboxylate. In some embodiments, the compound of Formula 1c is tert-butyl (2S,6R)-4-(2-((tert-butoxycarbonyl)(6-(4-(5-chloro-2-fluorophenyl)-1-((2-(trimethylsilyl)ethoxy)methyl)-1H-imidazol-5-yl)-1,5-naphthyridin-3-yl)amino)ethyl)-2,6-dimethylpiperazine-1-carboxylate or tert-butyl (2S,6R)-4-(2-((tert-butoxycarbonyl)(6-(5-(5-chloro-2-fluorophenyl)-1-((2-(trimethylsilyl)ethoxy)methyl)-1H-imidazol-4-yl)-1,5-naphthyridin-3-yl)amino)ethyl)-2,6-dimethylpiperazine-1-carboxylate. In some embodiments, the compound of Formula 1a is 7-bromo-2-(5-(5-chloro-2-fluorophenyl)-1-((2-(trimethylsilyl)ethoxy)methyl)-1H-imidazol-4-yl)-1,5-naphthyridine, the compound of Formula 1b is tert-butyl (2S,6R)-4-(2-((tert-butoxycarbonyl)amino)ethyl)-2,6-dimethylpiperazine-1-carboxylate, and the compound of Formula 1c is tert-butyl (2S,6R)-4-(2-((tert-butoxycarbonyl)(6-(5-(5-chloro-2-fluorophenyl)-1-((2-(trimethylsilyl)ethoxy)methyl)-1H-imidazol-4-yl)-1,5-naphthyridin-3-yl)amino)ethyl)-2,6-dimethylpiperazine-1-carboxylate.

In certain aspects, the present disclosure provides a method of making a crystalline form of a fumarate salt of the compound of Formula I, the method comprising: (a) combining the compound of Formula I, solvent and fumaric acid, thereby forming a mixture; (b) stirring the mixture; and (c) isolating the crystalline form from the mixture. In some embodiments, the molar ratio of the compound of Formula I to fumaric acid is between 1:1 and 1:1.5, such as 1:1.1. In some embodiments, fumaric acid is added to a slurry of the compound of Formula I in the solvent, optionally as a solution of fumaric acid in the solvent. In some embodiments, the mixture is heated, optionally to about 80° C. The mixture may be held at about 80° C., optionally for at least one hour or at least two hours. In some embodiments, water is added to the mixture before or after the stirring. The volume of the mixture may be reduced during the stirring, optionally by vacuum distillation. In some embodiments, after the stirring, the mixture is held between 0 and 60° C. for at least 8 hours, such as 8 to 72 hours. In some embodiments, the mixture is held at room temperature for 8 to 24 hours prior to the isolating. In some embodiments, the mixture is held at 50° C. for about 72 hours prior to the isolating. The crystalline compound may be isolated from the mixture by any conventional means, such as precipitation, filtration, concentration, centrifugation, dried in vacuo, and the like. Preferably, the crystalline compound is isolated from the mixture by filtration. In some embodiments, the solvent is selected from acetone, acetonitrile, ethyl acetate, methyl ethyl ketone, methanol, ethanol, 2-propanol, isobutanol, t-butanol, dichloromethane, 1,4-dioxane, isopropyl acetate, toluene, methyl t-butyl ether, cyclopentyl methyl ether, hexanes, water, and combinations thereof. In some embodiments, the solvent is a mixture selected from acetone with water, acetonitrile with water, ethanol with ethyl acetate, ethyl acetate with hexanes, and lower alcohols (e.g., C₁₋₆alkyl-OH) with water. In some embodiments, the solvent is a mixture of 2-propanol and water. In some embodiments, the solvent is 2-propanol. In some embodiments, the solvent is THF.

In certain aspects, the present disclosure provides a method of making a crystalline form of a fumarate salt of the compound of Formula I, the method comprising: (a) forming a slurry of the compound of Formula I, solvent and fumaric acid, optionally wherein the solvent is 2-propanol; (b) heating the slurry to at least 50° C., optionally at least 80° C.; (c) adding water to the slurry; (d) cooling the slurry to a reduced temperature, optionally to about 50° C., and holding the slurry at the reduced temperature for at least 12 hours, such as at least 24 hours, at least 36 hours, at least 48 hours, at least 60 hours, or at least 72 hours; and (e) isolating the crystalline form from the slurry, optionally by filtration. In some embodiments, the molar ratio of the compound of Formula I to fumaric acid is between 1:1 and 1:1.5, such as 1:1. In some embodiments, fumaric acid is added to a slurry of the compound of Formula I in the solvent. The crystalline compound may be isolated from the mixture by any conventional means, such as precipitation, filtration, concentration, centrifugation, dried in vacuo, and the like. In some embodiments, the solvent is selected from acetone, acetonitrile, ethyl acetate, methyl ethyl ketone, methanol, ethanol, 2-propanol, isobutanol, t-butanol, dichloromethane, 1,4-dioxane, isopropyl acetate, toluene, methyl t-butyl ether, cyclopentyl methyl ether, hexanes, water, and combinations thereof. In some embodiments, the solvent is a mixture selected from acetone with water, acetonitrile with water, ethanol with ethyl acetate, ethyl acetate with hexanes, and lower alcohols (e.g., C₁₋₆alkyl-OH) with water. In some embodiments, the solvent is a mixture of 2-propanol and water. In some embodiments, the solvent is 2-propanol.

In certain aspects, the present disclosure provides a method of making a crystalline form of a fumarate salt of the compound of Formula I, the method comprising: (a) dissolving fumaric acid in a mixture of solvent and water, optionally wherein the solvent is 2-propanol, thereby forming a fumaric acid solution; (b) adding the fumaric acid solution to a slurry of the compound of Formula I in solvent, optionally wherein the solvent is 2-propanol, thereby forming a crystallization slurry; (c) heating the crystallization slurry to an elevated temperature, optionally to at least 60° C., at least 70° C., or at least 80° C., thereby forming a crystallization solution; (d) removing a portion of the solvent while holding the crystallization solution at the elevated temperature±30° C., optionally via vacuum distillation, thereby forming a second slurry; (e) optionally, holding the second slurry at the elevated temperature for at least 10 minutes, at least 20 minutes, at least 30 minutes, at least 40 minutes, at least 50 minutes, or at least 60 minutes; (f) cooling the second slurry to about room temperature, optionally over at least 1 hour, at least 2 hours, at least 3 hours, at least 4 hours, or at least 5 hours; (g) holding the second slurry at about room temperature for at least 1 hour, at least 2 hours, at least 4 hours, at least 6 hours, at least 8 hours, at least 10 hours, or at least 12 hours; (h) isolating the crystalline form from the second slurry, optionally via filtration; (i) optionally, rinsing the crystalline form with solvent, optionally wherein the solvent is 2-propanol; and (j) optionally, drying the crystalline form. In some embodiments, the molar ratio of the compound of Formula I to fumaric acid is between 1:1 and 1:1.5, such as 1:1.1. The crystalline compound may be isolated from the mixture by any conventional means, such as precipitation, filtration, concentration, centrifugation, dried in vacuo, and the like. Preferably, the crystalline compound is isolated from the mixture by filtration. In some embodiments, the solvent is selected from acetone, acetonitrile, ethyl acetate, methyl ethyl ketone, methanol, ethanol, 2-propanol, isobutanol, t-butanol, dichloromethane, 1,4-dioxane, isopropyl acetate, toluene, methyl t-butyl ether, cyclopentyl methyl ether, hexanes, water, and combinations thereof. In some embodiments, the solvent is a mixture selected from acetone with water, acetonitrile with water, ethanol with ethyl acetate, ethyl acetate with hexanes, and lower alcohols (e.g., C₁₋₆alkyl-OH) with water. In some embodiments, the solvent is a mixture of 2-propanol and water. In some embodiments, the solvent is 2-propanol.

In some embodiments, the method further comprises, prior to (a): (a-1) dissolving a trihydrochloride salt of the compound of Formula I in water, thereby forming a salt solution; (a-2) adding to the salt solution a mixture of base and solvent, such as a mixture of aqueous NH₄OH and 2-methyltetrahydrofuran, optionally at about room temperature over at least 10 minutes, at least 20 minutes, at least 30 minutes, at least 40 minutes, at least 50 minutes, or at least 60 minutes, thereby forming a suspension; (a-3) optionally, heating the suspension to at least 30° C., at least 40° C., or at least 50° C.; (a-4) allowing the suspension to separate into two phases; (a-5) removing the aqueous phase; (a-6) optionally, adding additional solvent, such as 2-methyltetrahydrofuran, and reducing the volume of the resulting solution, optionally by vacuum distillation; (a-7) optionally, adding a metal scavenger, such as Silicycle siliametS thiol, to the solution and stirring for at least 30 minutes, at least 60 minutes, at least 90 minutes, at least 120 minutes, or at least 150 minutes, then removing the metal scavenger, optionally via filtration; (a-8) reducing the volume of the solution, optionally via vacuum distillation, thereby forming a concentrated solution; (a-9) diluting the concentrated solution with a second solvent, optionally wherein the second solvent is 2-propanol; and (a-10) reducing the volume of the solution, optionally via vacuum filtration, thereby forming the slurry of the compound of Formula I in solvent.

In certain aspects, the present disclosure provides a method of making a crystalline form of a fumarate salt of the compound of Formula I, the method comprising: (a) suspending the compound of Formula I and fumaric acid in a solvent, optionally wherein the solvent is THF; (b) stirring the suspension, optionally at about room temperature for at least 4 hours, at least 8 hours, at least 12 hours, at least 16 hours, at least 20 hours, or at least 24 hours; (c) isolating the crystalline form from the suspension, optionally via filtration; (d) optionally, rinsing the crystalline form with solvent, optionally wherein the solvent is THF; and (e) optionally, drying the crystalline form. In some embodiments, the molar ratio of the compound of Formula I to fumaric acid is between 1:1 and 1:1.5, such as 1:1.1. In some embodiments, fumaric acid is added to a slurry of the compound of Formula I in the solvent, optionally as a solution of fumaric acid in the solvent. The crystalline compound may be isolated from the mixture by any conventional means, such as precipitation, filtration, concentration, centrifugation, dried in vacuo, and the like. Preferably, the crystalline compound is isolated from the mixture by filtration. In some embodiments, the solvent is selected from acetone, acetonitrile, ethyl acetate, methyl ethyl ketone, methanol, ethanol, 2-propanol, isobutanol, t-butanol, dichloromethane, 1,4-dioxane, isopropyl acetate, toluene, methyl t-butyl ether, cyclopentyl methyl ether, hexanes, water, and combinations thereof. In some embodiments, the solvent is a mixture selected from acetone with water, acetonitrile with water, ethanol with ethyl acetate, ethyl acetate with hexanes, and lower alcohols (e.g., C₁₋₆alkyl-OH) with water. In some embodiments, the solvent is THF.

In certain aspects, the present disclosure provides a method of making a crystalline form of the compound of Formula I, the method comprising: (a) dissolving a salt of the compound of Formula I in water, thereby forming a salt solution, optionally wherein the salt is a trihydrochloride salt; (b) adding to the salt solution a mixture of base and solvent, such as a mixture of aqueous NH₄OH and 2-methyltetrahydrofuran, optionally at about room temperature over at least 1 minute, at least 2 minutes, at least 3 minutes, at least 4 minutes, at least 5 minutes, or at least 10 minutes, thereby forming a suspension; (c) optionally, heating the suspension to at least 30° C., at least 40° C., or at least 50° C. for at least 5 minutes, such as for at least 10 minutes; (d) allowing the suspension to separate into two phases; (e) removing the aqueous phase; (f) optionally, reducing the volume of the organic phase, optionally by vacuum distillation; (g) optionally, adding a second solvent, such as isopropanol, and reducing the volume of the resulting solution, optionally by vacuum distillation; (h) optionally, adding additional second solvent, such as isopropanol; (i) heating the solution to at least 30° C., at least 35° C., or at least 40° C. for at least 4 hours, at least 6 hours, at least 8 hours, at least 10 hours, or at least 12 hours, thereby forming a slurry; (j) optionally, cooling the slurry to room temperature and stirring for at least 12 hours, at least 24 hours, at least 36 hours, at least 48 hours, at least 60 hours, or at least 72 hours; and (k) isolating the crystalline form from the slurry, optionally via filtration; (l) optionally, rinsing the crystalline form with the second solvent, optionally wherein the second solvent is isopropanol; and (m) optionally, drying the crystalline form. In some embodiments, the molar ratio of the salt of the compound of Formula I to aqueous NH₄OH is between 1:1 and 1:5, such as about 1:3.5. The crystalline compound may be isolated from the mixture by any conventional means, such as precipitation, filtration, concentration, centrifugation, dried in vacuo, and the like. Preferably, the crystalline compound is isolated from the mixture by filtration. In some embodiments, the solvent and the second solvent are each independently selected from acetone, acetonitrile, ethyl acetate, methyl ethyl ketone, methanol, ethanol, isopropanol, isobutanol, t-butanol, dichloromethane, 1,4-dioxane, isopropyl acetate, toluene, methyl t-butyl ether, cyclopentyl methyl ether, hexanes, 2-methyltetrahydrofuran, water, and combinations thereof. In some embodiments, the solvent is 2-methyltetrahydrofuran. In some embodiments, the second solvent is isopropanol.

The polymorphs described herein are not limited by the starting materials used to produce the compound of Formula I. In some embodiments, a polymorph of a compound of Formula I or salt thereof is selected from Form I, Form II, Form III and mixtures thereof. In certain aspects, the present disclosure provides a crystalline form of a fumarate salt of the compound of Formula I, prepared according to a method described herein. In certain aspects, the present disclosure provides a crystalline form of a freebase of the compound of Formula I, prepared according to a method described herein. In certain aspects, the present disclosure provides a crystalline form prepared according to a method described herein.

Isolation and purification of the chemical entities and intermediates described herein can be effected, if desired, by any suitable separation or purification procedure, such as filtration, extraction, crystallization, column chromatography, thin-layer chromatography, thick-layer chromatography, high performance liquid chromatography, or a combination thereof. Specific illustrations of suitable separation and isolation procedures can be had by reference to the examples below. However, other equivalent separation or isolation procedures can also be used. Prior to crystallization, the compound of Formula I or salt thereof may be isolated in at least 50% chemical purity, such as at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% chemical purity. In some embodiments, the crystalline forms described herein are obtained by crystallizing a compound of Formula I or salt thereof having a chemical purity of less than about 99%, such as less than about 95%, less than about 90%, less than about 85%, less than about 80%, less than about 75%, or less than about 70%.

In some embodiments, a polymorph described herein, such as polymorph Form I, Form II or Form III, is stable at room temperature. In some embodiments, a polymorph described herein can be stored at room temperature for an extended period without significant chemical degradation or change in the crystalline form. In some embodiments, a polymorph described herein can be stored at room temperature for a period of at least 10 days, such as at least 30 days, at least 60 days, at least 90 days, or at least 120 days. A polymorph described herein may be stable at elevated temperature and/or high relative humidity (RH). For example, a polymorph described herein, such as polymorph Form I, Form II or Form III, can be stored at about 40° C. and 75% RH for at least 10 days-such as at least 30 days, at least 60 days, at least 90 days, or at least 120 days-without significant chemical degradation or change in the crystalline form. Preferably, after storage, the chemical purity of the compound of Formula I or salt thereof is at least 95%, such as at least 96%, at least 97%, at least 98%, or at least 99%. In some embodiments, at least 95%, such as at least 96%, at least 97%, at least 98%, or at least 99% of the compound of Formula I or salt thereof is in the same crystalline form as it was prior to storage.

Polymorph Form I

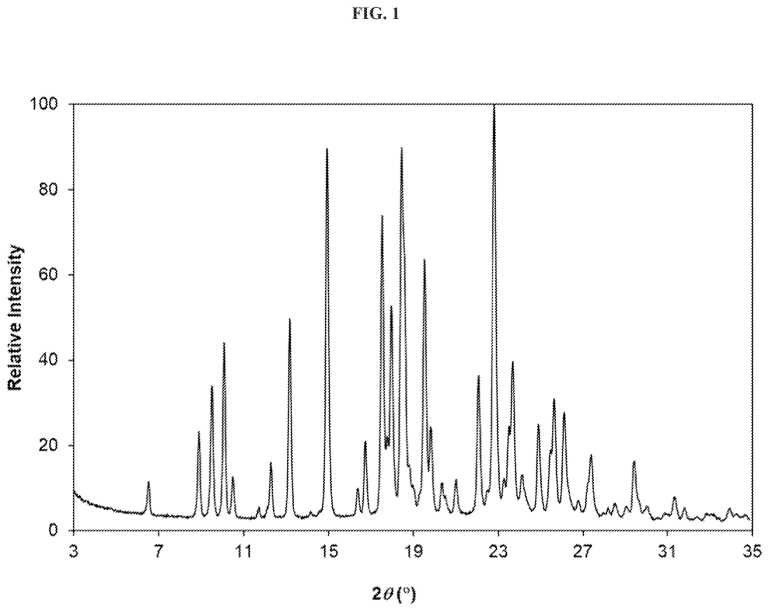

In certain aspects, the crystalline form of the compound of Formula I or salt thereof is polymorph Form I. Polymorph Form I is a mono-fumarate salt of the compound of Formula I. Polymorph Form I may be characterized by an X-ray powder diffraction pattern in which the peak positions are substantially in accordance with those shown in FIG. 1. Peaks with relative intensities greater than 1% in area are listed in Table 1. This pattern shows sharp diffraction peaks in the range of 5-35 degrees 2θ. These and other peaks in the diffraction pattern may be used to identify this form.

| Degrees 2θ | d(Å) | Area | Area % |
|---|---|---|---|
| 6.51 | 13.57 | 0.5 | 4.8 |
| 8.88 | 9.95 | 1.2 | 11.6 |
| 9.49 | 9.31 | 2 | 19.1 |
| 10.06 | 8.78 | 2.4 | 23.3 |
| 10.48 | 8.44 | 0.5 | 4.7 |
| 12.27 | 7.21 | 0.9 | 8.4 |
| 13.16 | 6.72 | 3 | 29.1 |
| 14.92 | 5.93 | 6.3 | 61.5 |
| 16.72 | 5.30 | 1 | 10.0 |
| 17.53 | 5.05 | 5 | 48.8 |
| 17.96 | 4.93 | 4.4 | 43.1 |
| 18.45 | 4.80 | 10.3 | 100.0 |
| 18.98 | 4.67 | 2 | 19.2 |
| 19.53 | 4.54 | 4.7 | 46.1 |
| 19.82 | 4.48 | 1.6 | 15.4 |
| 20.36 | 4.36 | 0.6 | 6.1 |
| 20.50 | 4.33 | 0.5 | 4.8 |
| 21.02 | 4.22 | 0.6 | 5.4 |
| 22.06 | 4.03 | 2.6 | 25.1 |
| 22.47 | 3.95 | 1.3 | 12.7 |
| 22.82 | 3.89 | 9 | 87.7 |
| 23.27 | 3.82 | 0.7 | 6.5 |
| 23.51 | 3.78 | 3.8 | 36.9 |
| 23.69 | 3.75 | 4.4 | 42.9 |
| 24.14 | 3.68 | 1.2 | 11.4 |
| 24.90 | 3.57 | 1.8 | 17.0 |
| 25.47 | 3.49 | 1.8 | 17.4 |
| 25.63 | 3.47 | 2.7 | 26.0 |
| 26.11 | 3.41 | 1.9 | 18.6 |

In some embodiments, polymorph Form I is characterized by an X-ray powder diffraction pattern comprising peaks at 13.2±0.2, 14.9±0.2 and 22.8±0.2 degrees 2θ. The X-ray powder diffraction pattern may further comprise at least one peak selected from 6.5±0.2, 8.9±0.2 and 17.5±0.2 degrees 2θ. In some embodiments, the X-ray powder diffraction pattern further comprises peaks at 6.5±0.2, 8.9±0.2 and 17.5±0.2 degrees 2θ. In some embodiments, the X-ray powder diffraction pattern further comprises at least one peak selected from 9.5±0.2, 10.1±0.2, 18.5±0.2 and 19.5±0.2 degrees 2θ. The X-ray powder diffraction pattern may further comprise peaks at 9.5±0.2, 10.1±0.2, 18.5±0.2 and 19.5±0.2 degrees 2θ. In some embodiments, polymorph Form I is characterized by an X-ray powder diffraction pattern comprising at least three peaks selected from 6.5±0.2, 8.9±0.2, 9.5±0.2, 10.1±0.2, 13.2±0.2, 14.9±0.2, 17.5±0.2, 18.5±0.2, 19.5±0.2 and 22.8±0.2 degrees 2θ. In some embodiments, polymorph Form I is characterized by an X-ray powder diffraction pattern comprising peaks at 6.5±0.2, 8.9±0.2, 9.5±0.2, 10.1±0.2, 13.2±0.2, 14.9±0.2, 17.5±0.2, 18.5±0.2, 19.5±0.2 and 22.8±0.2 degrees 2θ. In some embodiments, the X-ray powder diffraction pattern further comprises at least one peak selected from 10.5±0.2, 12.3±0.2, 16.7±0.2, 18.0±0.2, 19.0±0.2, 19.8±0.2, 20.4±0.2, 20.5±0.2, 21.0±0.2, 22.1±0.2, 22.5±0.2, 23.3±0.2, 23.5±0.2, 23.7±0.2, 24.1±0.2, 24.9±0.2, 25.5±0.2, 25.6±0.2 and 26.1±0.2 degrees 2θ.

In some embodiments, polymorph Form I is characterized by an X-ray powder diffraction pattern comprising peaks at 8.9±0.2, 9.5±0.2 and 10.1±0.2 degrees 2θ. The X-ray powder diffraction pattern may further comprise at least one peak selected from 6.5±0.2, 13.2±0.2 and 17.5±0.2 degrees 2θ. In some embodiments, the X-ray powder diffraction pattern further comprises peaks at 6.5±0.2, 13.2±0.2 and 17.5±0.2 degrees 2θ. In some embodiments, the X-ray powder diffraction pattern further comprises at least one peak selected from 14.9±0.2, 18.5±0.2, 19.5±0.2 and 22.8±0.2 degrees 2θ. The X-ray powder diffraction pattern may further comprise peaks at 14.9±0.2, 18.5±0.2, 19.5±0.2 and 22.8±0.2 degrees 2θ.

In some embodiments, polymorph Form I is characterized by an X-ray powder diffraction pattern comprising peaks at 6.5±0.2, 13.2±0.2 and 17.5±0.2 degrees 2θ. The X-ray powder diffraction pattern may further comprise at least one peak selected from 8.9±0.2, 9.5±0.2 and 22.8±0.2 degrees 2θ. In some embodiments, the X-ray powder diffraction pattern further comprises peaks at 8.9±0.2, 9.5±0.2 and 22.8±0.2 degrees 2θ. In some embodiments, the X-ray powder diffraction pattern further comprises at least one peak selected from 10.1±0.2, 14.9±0.2, 18.5±0.2 and 19.5±0.2 degrees 2θ. The X-ray powder diffraction pattern may further comprise peaks at 10.1±0.2, 14.9±0.2, 18.5±0.2 and 19.5±0.2 degrees 2θ.

In some embodiments, polymorph Form I is characterized by an X-ray powder diffraction pattern comprising peaks at 10.5±0.2, 12.3±0.2 and 13.2±0.2 degrees 2θ. The X-ray powder diffraction pattern may further comprise at least one peak selected from 14.9±0.2, 18.0±0.2 and 22.1±0.2 degrees 2θ. In some embodiments, the X-ray powder diffraction pattern further comprises peaks at 14.9±0.2, 18.0±0.2 and 22.1±0.2 degrees 2θ. In some embodiments, the X-ray powder diffraction pattern further comprises at least one peak selected from 8.9±0.2, 9.5±0.2 and 10.1±0.2 degrees 2θ. The X-ray powder diffraction pattern may further comprise peaks at 8.9±0.2, 9.5±0.2 and 10.1±0.2 degrees 2θ.

In some embodiments, polymorph Form I is characterized by an X-ray powder diffraction pattern comprising peaks at 6.5±0.2, 8.9±0.2, 9.5±0.2, 10.1±0.2, 10.5±0.2, 12.3±0.2, 13.2±0.2, 14.9±0.2, 16.7±0.2, 17.5±0.2, 18.0±0.2, 18.5±0.2, 19.0±0.2, 19.5±0.2, 19.8±0.2, 20.4±0.2, 20.5±0.2, 21.0±0.2, 22.1±0.2, 22.5±0.2, 22.8±0.2, 23.3±0.2, 23.5±0.2, 23.7±0.2, 24.1±0.2, 24.9±0.2, 25.5±0.2, 25.6±0.2 and 26.1±0.2 degrees 2θ.

Peak positions of XRPD patterns are relatively less sensitive to experimental parameters, such as sample preparation and instrument geometry, as compared to relative peak heights. The crystalline form may be characterized by an X-ray powder diffraction pattern in which the peak positions are substantially in accordance with the peak positions of the pattern shown in FIG. 1.

Polymorph Form I may also be identified by its characteristic DSC thermogram. As depicted in FIG. 2, an exemplary DSC thermogram of polymorph Form I recorded at a heating rate of 10° C. per minute exhibited a melting endotherm with an onset at about 260.0° C., a peak at about 263.0° C., and an area under the endotherm of 108.1 J/g. In some embodiments, polymorph Form I is characterized by a differential scanning calorimetry thermogram comprising an endotherm in the range of about 240 to 280° C., such as 245-275° C., 250-270° C., 255-270° C., 258-268° C., 259-267° C., 260-266° C., 261-265° C. or 262-264° C. In some embodiments, polymorph Form I is characterized by a differential scanning calorimetry thermogram comprising an endotherm at about 260-266° C., such as about 263° C. In some embodiments, polymorph Form I is characterized by a differential scanning calorimetry thermogram comprising an endotherm at about 263° C. In some embodiments, polymorph Form I is characterized by a differential scanning calorimetry thermogram which shows a maximum in endothermic heat flow at a temperature of about 263.0±3° C., such as about 263.0±2° C. or 263.0±1° C. In some embodiments, polymorph Form I is characterized by a differential scanning calorimetry thermogram substantially in accordance with that shown in FIG. 2.

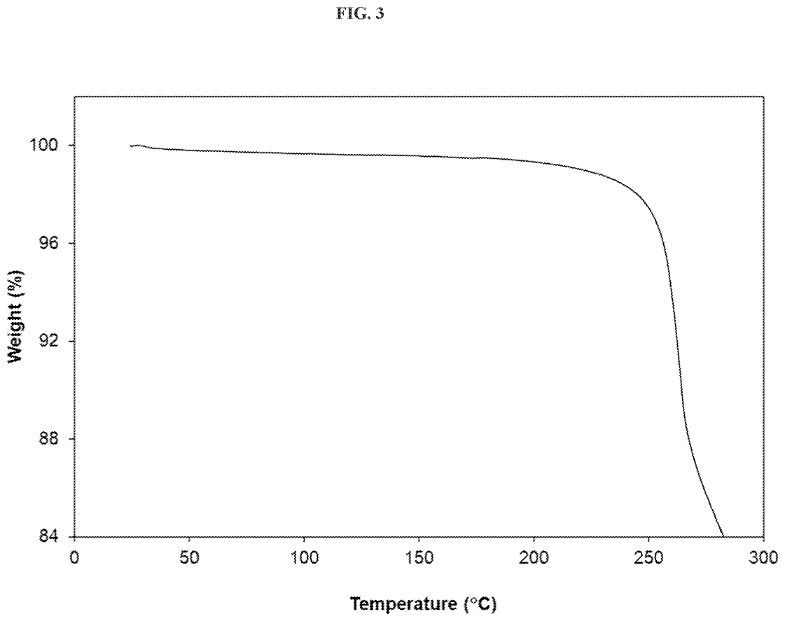

Polymorph Form I may also be identified by its characteristic TGA profile, an exemplary example of which is depicted in FIG. 3. In some embodiments, polymorph Form I shows less than 3% mass loss until greater than about 250° C. Polymorph Form I decomposes after melting, as evidenced by significant weight loss occurring at an onset of about 254° C.

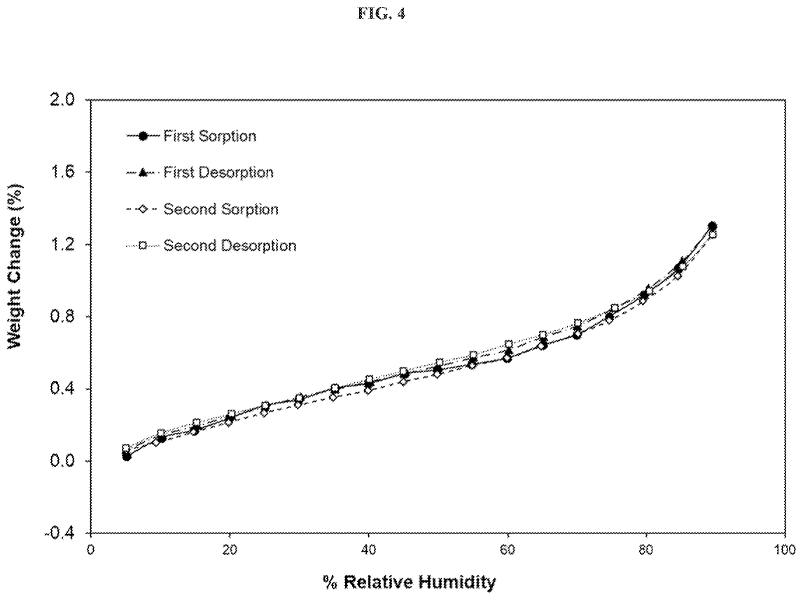

In some embodiments, polymorph Form I is characterized by the DMS isotherm depicted in FIG. 4. The total moisture gain observed is about 1.30% by weight when exposed to 5-90% relative humidity. The solid obtained after two consecutive sorption-desorption cycles showed the same XRPD pattern as the starting material, indicating no change in form after this experiment. These data indicate that polymorph Form I does not convert to a hydrated form in the presence of water and is slightly-hygroscopic.

Polymorph Form I may be identified by microcrystal electron diffraction (MicroED) analysis. Unit cell parameters and space group details are provided in Table 2. Data were collected on a Thermo Fisher Scientific Glacios Cryo Transmission Electron Microscope (Cryo-TEM) operated at 200 kV, equipped with a Ceta-D detector and operated at cryogenic temperature (below −170° C.). The data were collected at a dose rate of approximately 0.1 e−/s/A² and the datasets were indexed, refined, integrated and scaled with the program DIALS (Diffraction Integration for Advanced Light Sources). Hydrogen atoms were modeled with interatomic distances based on values derived from neutron scattering and also modeled as riding.

TABLE 2

| | |
|---|---|
| Temperature of Data Collection | −193° C. |
| Space group | P2₁/n |
| Unit cell dimensions | a = 7.8879(16) Å |
| | b = 18.276(4) Å |
| | c = 19.959(4) Å |
| | α = 90° |
| | β = 94.34(3)° |
| | γ = 90° |

In some embodiments, polymorph Form I is characterized by microcrystal electron diffraction having a P2₁/n space group. A single crystal of polymorph Form I may comprise the following unit cell dimensions: a=7.89±0.10 Å; b=18.28±0.10 Å; c=19.96±0.10 Å; α=90±0.1°; β=94.3±0.1°; and γ=90±0.1°. In some embodiments, a single crystal of polymorph Form I comprises the following unit cell dimensions: a=7.89±0.30 Å; b=18.28±0.30 Å; c=19.96±0.30 Å; α=90±0.3°; β=94.3±0.3°; and γ=90±0.3°.

In some embodiments, the chemical purity of polymorph Form I is greater than 60%, such as greater than 70%, 80%, 90%, 95% or 99%. In some embodiments, the chemical purity of polymorph Form I is greater than about 90%. In some embodiments, the chemical purity of polymorph Form I is greater than about 95%. In some embodiments, the chemical purity of polymorph Form I is greater than about 99%. The chemical purity of polymorph Form I may be measured by any appropriate analytical technique, such as HPLC analysis.

In some embodiments, polymorph Form I is stable at room temperature. Polymorph Form I may be stored at room temperature for an extended period of time-such as at least 10 days, at least 30 days, at least 60 days, at least 90 days, or at least 120 days-without significant chemical degradation or change in crystalline form. In some embodiments, the chemical purity of polymorph Form I is at least 95%, such as at least 98%, after 30 days of storage at room temperature. In some embodiments, the chemical purity of polymorph Form I is at least 95%, such as at least 98%, after 120 days of storage at room temperature. In some embodiments, polymorph Form I is stable at elevated temperatures and/or relative humidity (RH). For example, polymorph Form I may be stored at about 40° C. and about 75% RH for an extended period of time-such as at least 10 days, at least 30 days, at least 60 days, at least 90 days, or at least 120 days-without significant chemical degradation or change in crystalline form. In some embodiments, the chemical purity of polymorph Form I is at least 95%, such as at least 98%, after 30 days of storage at 40° C. and 75% RH. In some embodiments, the chemical purity of polymorph Form I is at least 95%, such as at least 98%, after 120 days of storage at 40° C. and 75% RH. In some embodiments, polymorph Form I may be stored at about 60° C. for an extended period of time-such as at least 10 days, at least 30 days, at least 60 days, at least 90 days, or at least 120 days-without significant chemical degradation or change in crystalline form. In some embodiments, the chemical purity of polymorph Form I is at least 95%, such as at least 98%, after 30 days of storage at 60° C. In some embodiments, the chemical purity of polymorph Form I is at least 95%, such as at least 98%, after 120 days of storage at 60° C.

Polymorph Form II

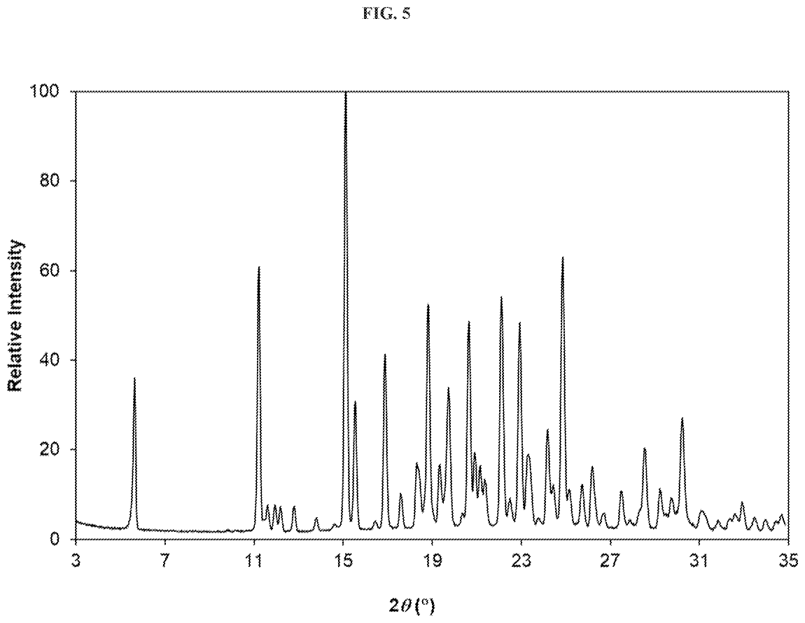

In certain aspects, the crystalline form of the compound of Formula I or salt thereof is polymorph Form II. Polymorph Form II is a mono-fumarate salt of the compound of Formula I. Polymorph Form II may be characterized by an X-ray powder diffraction pattern in which the peak positions are substantially in accordance with those shown in FIG. 5. Peaks with relative intensities greater than 1% in area are listed in Table 3. This pattern shows sharp diffraction peaks in the range of 5-35 degrees 2θ. These and other peaks in the diffraction pattern may be used to identify this form.

TABLE 3

| Degrees 2θ | d(Å) | Area | Area % |
|---|---|---|---|
| 5.61 | 15.74 | 137.6 | 40.1 |
| 11.18 | 7.91 | 199.9 | 58.2 |
| 11.57 | 7.64 | 17.8 | 5.2 |
| 11.92 | 7.42 | 12.6 | 3.7 |
| 12.14 | 7.28 | 16.5 | 4.8 |
| 12.76 | 6.93 | 18.4 | 5.4 |
| 15.08 | 5.87 | 343.3 | 100 |
| 15.49 | 5.71 | 98.7 | 28.8 |
| 16.86 | 5.26 | 133.8 | 39 |
| 17.57 | 5.04 | 28.2 | 8.2 |
| 18.78 | 4.72 | 154.7 | 45.1 |
| 19.33 | 4.59 | 43.7 | 12.7 |
| 19.71 | 4.50 | 131.0 | 38.2 |
| 20.63 | 4.30 | 169.2 | 49.3 |
| 20.89 | 4.25 | 36.5 | 10.6 |
| 22.06 | 4.03 | 180.0 | 52.4 |
| 22.92 | 3.88 | 156.3 | 45.5 |
| 23.26 | 3.82 | 45.5 | 13.2 |
| 24.39 | 3.65 | 31.8 | 9.3 |
| 24.82 | 3.58 | 266.9 | 77.7 |
| 25.71 | 3.46 | 30.3 | 8.8 |
| 26.14 | 3.41 | 51.2 | 14.9 |
| 27.43 | 3.25 | 26.8 | 7.8 |
| 28.51 | 3.13 | 79.4 | 23.1 |
| 29.22 | 3.05 | 41.9 | 12.2 |
| 29.70 | 3.01 | 28.8 | 8.4 |
| 30.20 | 2.96 | 117.1 | 34.1 |

In some embodiments, polymorph Form II is characterized by an X-ray powder diffraction pattern comprising peaks at 5.6±0.2, 11.2±0.2 and 15.5±0.2 degrees 2θ. The X-ray powder diffraction pattern may further comprise at least one peak selected from 18.8±0.2, 20.6±0.2 and 22.9±0.2 degrees 2θ. In some embodiments, the X-ray powder diffraction pattern further comprises peaks at 18.8±0.2, 20.6±0.2 and 22.9±0.2 degrees 2θ. In some embodiments, the X-ray powder diffraction pattern further comprises at least one peak selected from 15.1±0.2, 22.1±0.2 and 24.8±0.2 degrees 2θ. The X-ray powder diffraction pattern may further comprise peaks at 15.1±0.2, 22.1±0.2 and 24.8±0.2 degrees 2θ. In some embodiments, polymorph Form II is characterized by an X-ray powder diffraction pattern comprising at least three peaks selected from 5.6±0.2, 11.2±0.2, 15.1±0.2, 15.5±0.2, 18.8±0.2, 20.6±0.2, 22.1±0.2, 22.9±0.2 and 24.8±0.2 degrees 2θ. In some embodiments, polymorph Form II is characterized by an X-ray powder diffraction pattern comprising peaks at 5.6±0.2, 11.2±0.2, 15.1±0.2, 15.5±0.2, 18.8±0.2, 20.6±0.2, 22.1±0.2, 22.9±0.2 and 24.8±0.2 degrees 2θ. In some embodiments, the X-ray powder diffraction pattern further comprises at least one peak selected from 11.6±0.2, 11.9±0.2, 12.1±0.2, 12.8±0.2, 16.9±0.2, 17.6±0.2, 19.3±0.2, 19.7±0.2, 20.9±0.2, 23.3±0.2, 24.4±0.2, 25.7±0.2, 26.1±0.2, 27.4±0.2, 28.5±0.2, 29.2±0.2, 29.7±0.2 and 30.2±0.2 degrees 2θ.

In some embodiments, polymorph Form II is characterized by an X-ray powder diffraction pattern comprising peaks at 11.2±0.2, 15.1±0.2 and 24.8±0.2 degrees 2θ. The X-ray powder diffraction pattern may further comprise at least one peak selected from 5.6±0.2, 18.8±0.2 and 22.1±0.2 degrees 2θ. In some embodiments, the X-ray powder diffraction pattern further comprises peaks at 5.6±0.2, 18.8±0.2 and 22.1±0.2 degrees 2θ. In some embodiments, the X-ray powder diffraction pattern further comprises at least one peak selected from 15.5±0.2, 20.6±0.2 and 22.9±0.2 degrees 2θ. The X-ray powder diffraction pattern may further comprise peaks at 15.5±0.2, 20.6±0.2 and 22.9±0.2 degrees 2θ.

In some embodiments, polymorph Form II is characterized by an X-ray powder diffraction pattern comprising peaks at 15.5±0.2, 20.6±0.2 and 22.1±0.2 degrees 2θ. The X-ray powder diffraction pattern may further comprise at least one peak selected from 11.2±0.2, 15.1±0.2 and 22.9±0.2 degrees 2θ. In some embodiments, the X-ray powder diffraction pattern further comprises peaks at 11.2±0.2, 15.1±0.2 and 22.9±0.2 degrees 2θ. In some embodiments, the X-ray powder diffraction pattern further comprises at least one peak selected from 5.6±0.2, 18.8±0.2 and 24.8±0.2 degrees 2θ. The X-ray powder diffraction pattern may further comprise peaks at 5.6±0.2, 18.8±0.2 and 24.8±0.2 degrees 2θ.

In some embodiments, polymorph Form II is characterized by an X-ray powder diffraction pattern comprising peaks at 15.1±0.2, 16.9±0.2 and 19.7±0.2 degrees 2θ. The X-ray powder diffraction pattern may further comprise at least one peak selected from 5.6±0.2, 11.2±0.2 and 24.8±0.2 degrees 2θ. In some embodiments, the X-ray powder diffraction pattern further comprises peaks at 5.6±0.2, 11.2±0.2 and 24.8±0.2 degrees 2θ. In some embodiments, the X-ray powder diffraction pattern further comprises at least one peak selected from 15.5±0.2, 18.8±0.2, 20.6±0.2, 22.1±0.2 and 22.9±0.2 degrees 2θ. The X-ray powder diffraction pattern may further comprise peaks at 15.5±0.2, 18.8±0.2, 20.6±0.2, 22.1±0.2 and 22.9±0.2 degrees 2θ.

In some embodiments, polymorph Form II is characterized by an X-ray powder diffraction pattern comprising peaks at 5.6±0.2, 11.2±0.2, 11.6±0.2, 11.9±0.2, 12.1±0.2, 12.8±0.2, 15.1±0.2, 15.5±0.2, 16.9±0.2, 17.6±0.2, 18.8±0.2, 19.3±0.2, 19.7±0.2, 20.6±0.2, 20.9±0.2, 22.1±0.2, 22.9±0.2, 23.3±0.2, 24.4±0.2, 24.8±0.2, 25.7±0.2, 26.1±0.2, 27.4±0.2, 28.5±0.2, 29.2±0.2, 29.7±0.2 and 30.2±0.2 degrees 2θ.

Peak positions of XRPD patterns are relatively less sensitive to experimental parameters, such as sample preparation and instrument geometry, as compared to relative peak heights. The crystalline form may be characterized by an X-ray powder diffraction pattern in which the peak positions are substantially in accordance with the peak positions of the pattern shown in FIG. 5.

Polymorph Form II may also be identified by its characteristic DSC thermogram. As depicted in FIG. 6, an exemplary DSC thermogram of polymorph Form II recorded at a heating rate of 10° C. per minute exhibited a melting endotherm with an onset at about 259.2° C., a peak at about 263.2° C., and an area under the endotherm of 115.9 J/g. In some embodiments, polymorph Form II is characterized by a differential scanning calorimetry thermogram comprising an endotherm in the range of about 240 to 280° C., such as 245-275° C., 250-270° C., 255-270° C., 258-268° C., 259-267° C., 260-266° C., 261-265° C. or 262-264° C. In some embodiments, polymorph Form II is characterized by a differential scanning calorimetry thermogram comprising an endotherm at about 259-267° C., such as about 263° C. In some embodiments, polymorph Form II is characterized by a differential scanning calorimetry thermogram comprising an endotherm at about 263° C. In some embodiments, polymorph Form II is characterized by a differential scanning calorimetry thermogram which shows a maximum in endothermic heat flow at a temperature of about 263.2±3° C., such as about 263.2±2° C. or 263.2±1° C. In some embodiments, polymorph Form II is characterized by a differential scanning calorimetry thermogram substantially in accordance with that shown in FIG. 6.

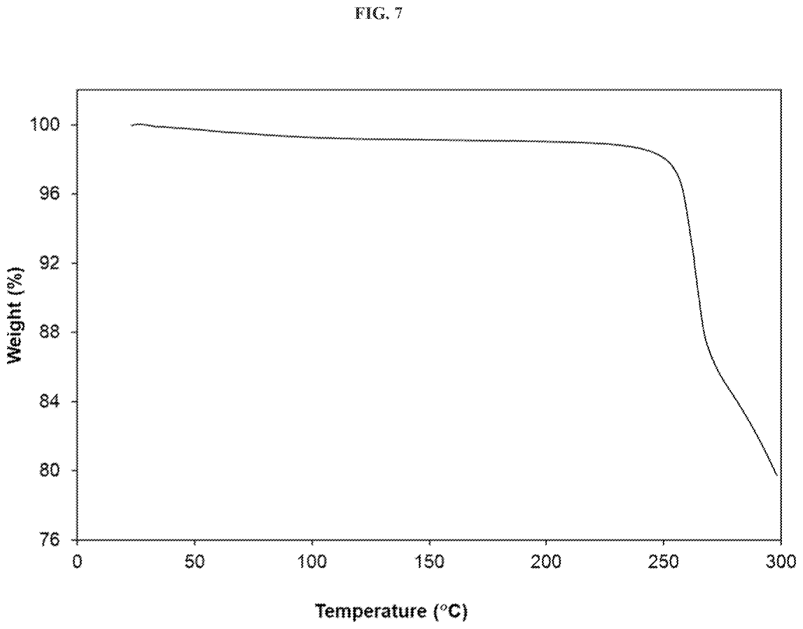

Polymorph Form II may also be identified by its characteristic TGA profile, an exemplary example of which is depicted in FIG. 7. In some embodiments, polymorph Form II shows less than 3% mass loss until greater than about 250° C. Polymorph Form II decomposes after melting, as evidenced by significant weight loss occurring at an onset of about 255° C.

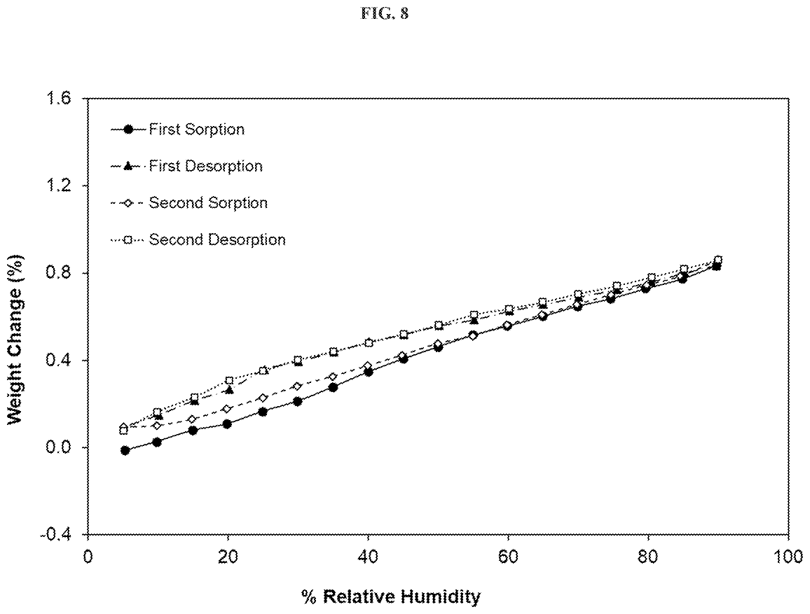

In some embodiments, polymorph Form II is characterized by the DMS isotherm depicted in FIG. 8. The total moisture gain observed is about 0.85% by weight when exposed to 5-90% relative humidity. The solid obtained after two consecutive sorption-desorption cycles showed the same XRPD pattern as the starting material, indicating no change in form after this experiment. These data indicate that polymorph Form II does not convert to a hydrated form in the presence of water and is slightly-hygroscopic. Polymorph Form II may therefore be characterized as anhydrous, slightly-hygroscopic or both.

Polymorph Form II may be identified by single crystal X-ray diffraction analysis. Unit cell parameters and crystal system and space group details are provided in Table 4. Data were collected on a Rigaku Atlas CCD diffractometer equipped with an Oxford Cryosystems Cobra cooling device. The data were collected using Cu-Kα radiation and the crystal structure was solved and refined using the Bruker AXS SHELXTL software. Hydrogen atoms attached to carbon were placed geometrically and allowed to refine with a riding isotropic displacement parameter. Hydrogen atoms attached to the heteroatoms were located in a difference Fourier map and were allowed to refine freely with an isotropic displacement parameter.

TABLE 4

| | |
|---|---|
| Temperature of Data Collection | 293(2) K |
| Wavelength used for Data Collection | 1.54184 Å |
| Crystal system | Triclinic |
| Space group | P-1 |
| Unit cell dimensions | a = 9.4166(2) Å |
| | b = 10.0663(2) Å |
| | c = 16.3436(4) Å |
| | α = 75.490(2)° |
| | β = 87.324(2)° |
| | γ = 73.755(2)° |
| Unit cell volume | 1439.46(6) Å³ |
| Final R indices [F² > 2σ(F²)] | R₁ = 0.0394, wR₂ = 0.1049 |

In some embodiments, polymorph Form II is characterized by single crystal X-ray diffraction having a P-1 space group. A single crystal of polymorph Form II may comprise the following unit cell dimensions: a=9.42±0.10 Å; b=10.07±0.10 Å; c=16.34±0.10 Å; α=75.5±0.1°; β=87.3±0.1°; and γ=73.8±0.1°. In some embodiments, a single crystal of polymorph Form II comprises the following unit cell dimensions: a=9.42±0.30 Å; b=10.07±0.30 Å; c=16.34±0.30 Å; α=75.5±0.3°; β=87.3±0.3°; and γ=73.8±0.3°.

In some embodiments, the chemical purity of polymorph Form II is greater than 60%, such as greater than 70%, 80%, 90%, 95% or 99%. In some embodiments, the chemical purity of polymorph Form II is greater than about 90%. In some embodiments, the chemical purity of polymorph Form II is greater than about 95%. In some embodiments, the chemical purity of polymorph Form II is greater than about 99%. The chemical purity of polymorph Form II may be measured by any appropriate analytical technique, such as HPLC analysis.

In some embodiments, polymorph Form II is stable at room temperature. Polymorph Form II may be stored at room temperature for an extended period of time-such as at least 10 days, at least 30 days, at least 60 days, at least 90 days, or at least 120 days-without significant chemical degradation or change in crystalline form. In some embodiments, the chemical purity of polymorph Form II is at least 95%, such as at least 98%, after 30 days of storage at room temperature. In some embodiments, the chemical purity of polymorph Form II is at least 95%, such as at least 98%, after 120 days of storage at room temperature. In some embodiments, polymorph Form II is stable at elevated temperatures and/or relative humidity (RH). For example, polymorph Form II may be stored at about 40° C. and about 75% RH for an extended period of time-such as at least 10 days, at least 30 days, at least 60 days, at least 90 days, or at least 120 days-without significant chemical degradation or change in crystalline form. In some embodiments, the chemical purity of polymorph Form II is at least 95%, such as at least 98%, after 30 days of storage at 40° C. and 75% RH. In some embodiments, the chemical purity of polymorph Form II is at least 95%, such as at least 98%, after 120 days of storage at 40° C. and 75% RH. In some embodiments, polymorph Form II may be stored at about 60° C. for an extended period of time-such as at least 10 days, at least 30 days, at least 60 days, at least 90 days, or at least 120 days-without significant chemical degradation or change in crystalline form. In some embodiments, the chemical purity of polymorph Form II is at least 95%, such as at least 98%, after 30 days of storage at 60° C. In some embodiments, the chemical purity of polymorph Form II is at least 95%, such as at least 98%, after 120 days of storage at 60° C.

Polymorph Form III

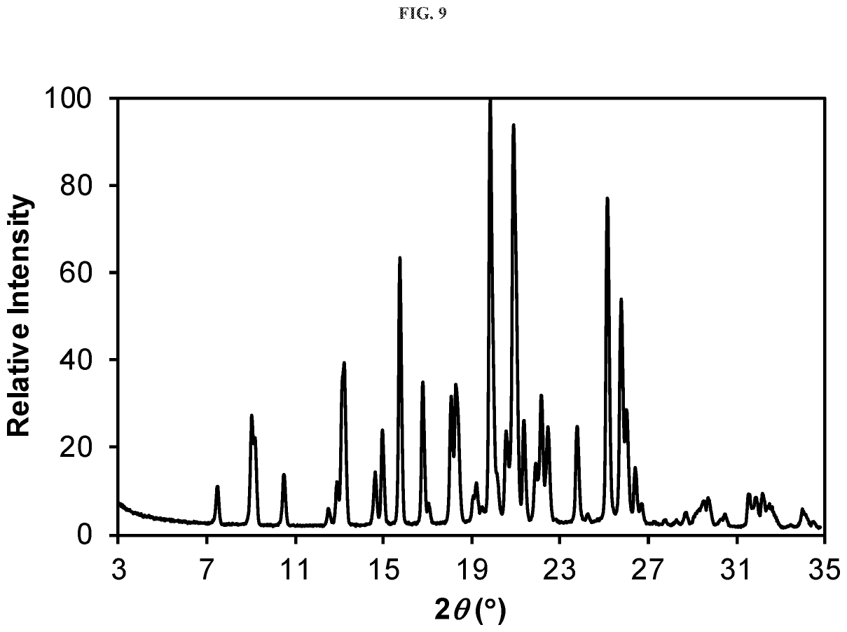

In certain aspects, the crystalline form of the compound of Formula I or salt thereof is polymorph Form III. Polymorph Form III is a freebase of the compound of Formula I. Polymorph Form III may be characterized by an X-ray powder diffraction pattern in which the peak positions are substantially in accordance with those shown in FIG. 9. Certain peaks with relative intensities greater than 1% in area are listed in Table 5. This pattern shows sharp diffraction peaks in the range of 5-35 degrees 2θ. These and other peaks in the diffraction pattern may be used to identify this form.

TABLE 5

| Degrees 2θ | d(Å) | Area |
|---|---|---|
| 7.48 | 12.67 | 0.7 |
| 9.04 | 10.49 | 3.2 |
| 9.19 | 10.32 | 5.1 |
| 10.50 | 9.03 | 1.0 |
| 13.23 | 7.18 | 4.3 |
| 14.63 | 6.49 | 1.1 |
| 14.96 | 6.35 | 1.6 |
| 15.76 | 6.03 | 4.5 |
| 16.79 | 5.66 | 2.6 |
| 18.08 | 5.26 | 2.5 |
| 18.29 | 5.20 | 3.5 |
| 19.85 | 4.80 | 10.4 |
| 20.91 | 4.55 | 10.5 |
| 22.16 | 4.30 | 2.7 |
| 23.79 | 4.01 | 2 |
| 25.15 | 3.80 | 6.7 |
| 25.78 | 3.70 | 4.6 |

In some embodiments, polymorph Form III is characterized by an X-ray powder diffraction pattern comprising peaks at 10.5±0.2, 15.8±0.2 and 25.2±0.2 degrees 2θ. The X-ray powder diffraction pattern may further comprise at least one peak selected from 7.5±0.2, 19.9±0.2 and 20.9±0.2 degrees 2θ. In some embodiments, the X-ray powder diffraction pattern further comprises peaks at 7.5±0.2, 19.9±0.2 and 20.9±0.2 degrees 2θ. In some embodiments, the X-ray powder diffraction pattern further comprises at least one peak selected from 9.0±0.2, 13.2±0.2, 16.8±0.2 and 25.8±0.2 degrees 2θ. The X-ray powder diffraction pattern may further comprise peaks at 9.0±0.2, 13.2±0.2, 16.8±0.2 and 25.8±0.2 degrees 2θ. In some embodiments, polymorph Form III is characterized by an X-ray powder diffraction pattern comprising at least three peaks selected from 7.5±0.2, 9.0±0.2, 10.5±0.2, 13.2±0.2, 15.8±0.2, 16.8±0.2, 19.9±0.2, 20.9±0.2, 25.2±0.2 and 25.8±0.2 degrees 2θ. In some embodiments, polymorph Form III is characterized by an X-ray powder diffraction pattern comprising peaks at 7.5±0.2, 9.0±0.2, 10.5±0.2, 13.2±0.2, 15.8±0.2, 16.8±0.2, 19.9±0.2, 20.9±0.2, 25.2±0.2 and 25.8±0.2 degrees 2θ. In some embodiments, the X-ray powder diffraction pattern further comprises at least one peak selected from 9.2±0.2, 14.6±0.2, 15.0±0.2, 18.1±0.2, 18.3±0.2, 22.2±0.2 and 23.8±0.2 degrees 2θ.

In some embodiments, polymorph Form III is characterized by an X-ray powder diffraction pattern comprising peaks at 19.9±0.2, 20.9±0.2 and 25.2±0.2 degrees 2θ. The X-ray powder diffraction pattern may further comprise at least one peak selected from 13.2±0.2, 15.8±0.2 and 25.8±0.2 degrees 2θ. In some embodiments, the X-ray powder diffraction pattern further comprises peaks at 13.2±0.2, 15.8±0.2 and 25.8±0.2 degrees 2θ. In some embodiments, the X-ray powder diffraction pattern further comprises at least one peak selected from 7.5±0.2, 9.0±0.2, 10.5±0.2 and 16.8±0.2 degrees 2θ. The X-ray powder diffraction pattern may further comprise peaks at 7.5±0.2, 9.0±0.2, 10.5±0.2 and 16.8±0.2 degrees 2θ.

In some embodiments, polymorph Form III is characterized by an X-ray powder diffraction pattern comprising peaks at 7.5±0.2, 15.8±0.2 and 25.2±0.2 degrees 2θ. The X-ray powder diffraction pattern may further comprise at least one peak selected from 9.0±0.2, 13.2±0.2 and 20.9±0.2 degrees 2θ. In some embodiments, the X-ray powder diffraction pattern further comprises peaks at 9.0±0.2, 13.2±0.2 and 20.9±0.2 degrees 2θ. In some embodiments, the X-ray powder diffraction pattern further comprises at least one peak selected from 10.5±0.2, 16.8±0.2, 19.9±0.2 and 25.8±0.2 degrees 2θ. The X-ray powder diffraction pattern may further comprise peaks at 10.5±0.2, 16.8±0.2, 19.9±0.2 and 25.8±0.2 degrees 2θ.

In some embodiments, polymorph Form III is characterized by an X-ray powder diffraction pattern comprising peaks at 9.0±0.2, 16.8±0.2 and 25.8±0.2 degrees 2θ. The X-ray powder diffraction pattern may further comprise at least one peak selected from 7.5±0.2, 10.5±0.2 and 19.9±0.2 degrees 2θ. In some embodiments, the X-ray powder diffraction pattern further comprises peaks at 7.5±0.2, 10.5±0.2 and 19.9±0.2 degrees 2θ. In some embodiments, the X-ray powder diffraction pattern further comprises at least one peak selected from 13.2±0.2, 15.8±0.2 and 20.9±0.2 degrees 2θ. The X-ray powder diffraction pattern may further comprise peaks at 13.2±0.2, 15.8±0.2 and 20.9±0.2 degrees 2θ.

In some embodiments, polymorph Form III is characterized by an X-ray powder diffraction pattern comprising peaks at 7.5±0.2, 9.0±0.2, 9.2±0.2, 10.5±0.2, 13.2±0.2, 14.6±0.2, 15.0±0.2, 15.8±0.2, 16.8±0.2, 18.1±0.2, 18.3±0.2, 19.9±0.2, 20.9±0.2, 22.2±0.2, 23.8±0.2, 25.2±0.2 and 25.8±0.2 degrees 2θ.

Peak positions of XRPD patterns are relatively less sensitive to experimental parameters, such as sample preparation and instrument geometry, as compared to relative peak heights. The crystalline form may be characterized by an X-ray powder diffraction pattern in which the peak positions are substantially in accordance with the peak positions of the pattern shown in FIG. 9.

Polymorph Form III may also be identified by its characteristic DSC thermogram. As depicted in FIG. 10, an exemplary DSC thermogram of polymorph Form III recorded at a heating rate of 10° C. per minute exhibited a melting endotherm with an onset at about 224.0° C., a peak at about 224.8° C., and an area under the endotherm of 168.4 J/g, and a minor pre-melting endotherm having an onset at about 134.8° C., a peak at about 204.2° C., and an area under the endotherm of 3.9 J/g. In some embodiments, polymorph Form III is characterized by a differential scanning calorimetry thermogram comprising an endotherm in the range of about 200 to 240° C., such as 205-235° C., 210-235° C., 215-230° C., 220-230° C., 221-229° C., 222-228° C., 223-227° C. or 224-226° C. In some embodiments, polymorph Form III is characterized by a differential scanning calorimetry thermogram comprising an endotherm at about 221-229° C., such as about 225° C. In some embodiments, polymorph Form III is characterized by a differential scanning calorimetry thermogram comprising an endotherm at about 225° C. In some embodiments, polymorph Form III is characterized by a differential scanning calorimetry thermogram which shows a maximum in endothermic heat flow at a temperature of about 224.8±3° C., such as about 224.8±2° C. or 224.8±1° C. In some embodiments, polymorph Form III is characterized by a differential scanning calorimetry thermogram substantially in accordance with that shown in FIG. 10.

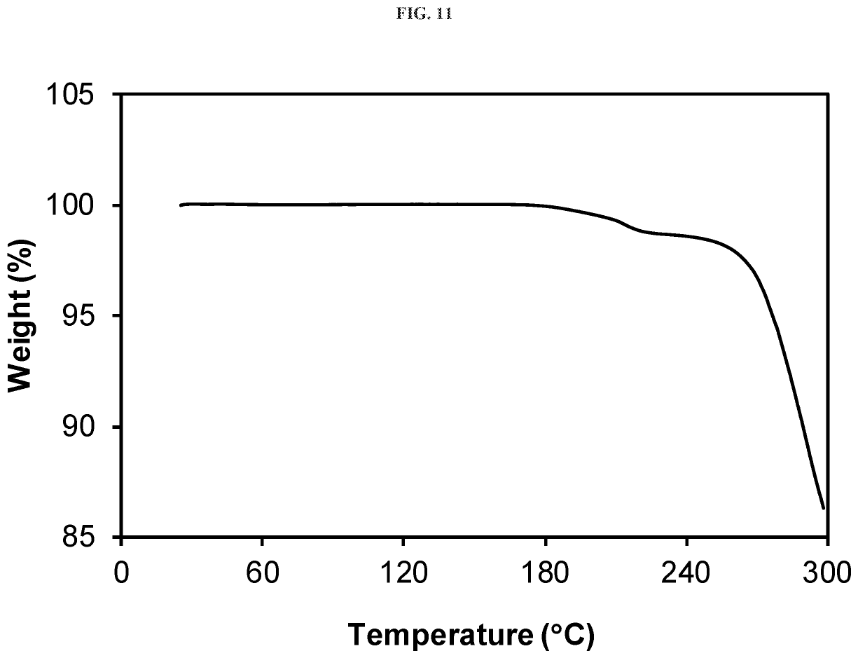

Polymorph Form III may also be identified by its characteristic TGA profile, an exemplary example of which is depicted in FIG. 11. In some embodiments, polymorph Form III shows less than 3% mass loss until greater than about 242° C. Polymorph Form III decomposes after melting, as evidenced by significant weight loss occurring at an onset of about 260° C.

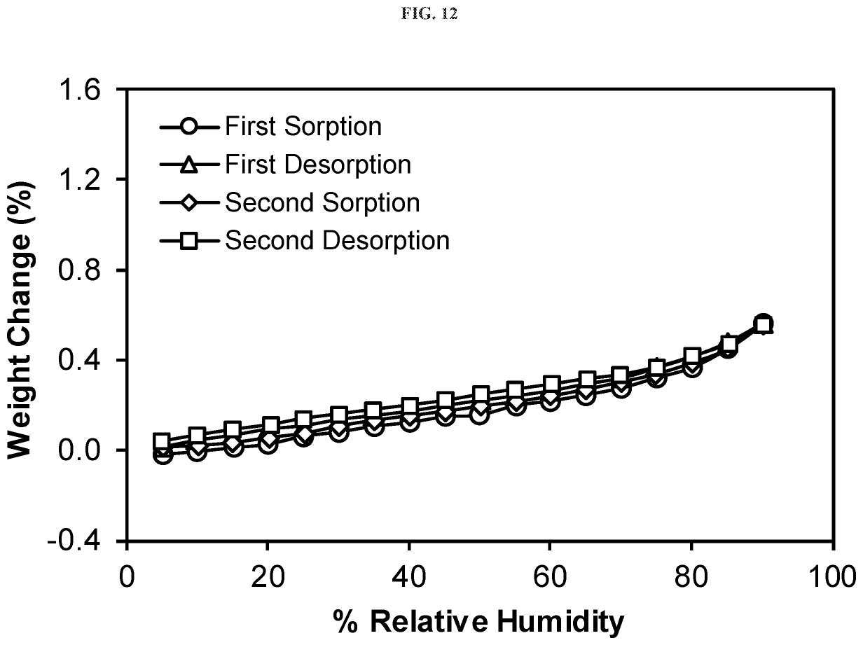

In some embodiments, polymorph Form III is characterized by the DMS isotherm depicted in FIG. 12. The total moisture gain observed is about 0.6% by weight when exposed to 5-90% relative humidity. The solid obtained after two consecutive sorption-desorption cycles showed the same XRPD pattern as the starting material, indicating no change in form after this experiment. These data indicate that polymorph Form III does not convert to a hydrated form in the presence of water and is slightly-hygroscopic.

In some embodiments, the chemical purity of polymorph Form III is greater than 60%, such as greater than 70%, 80%, 90%, 95% or 99%. In some embodiments, the chemical purity of polymorph Form III is greater than about 90%. In some embodiments, the chemical purity of polymorph Form III is greater than about 95%. In some embodiments, the chemical purity of polymorph Form III is greater than about 99%. The chemical purity of polymorph Form III may be measured by any appropriate analytical technique, such as HPLC analysis.

In some embodiments, polymorph Form III is stable at room temperature. Polymorph Form III may be stored at room temperature for an extended period of time-such as at least 10 days, at least 30 days, at least 60 days, at least 90 days, or at least 120 days-without significant chemical degradation or change in crystalline form. In some embodiments, the chemical purity of polymorph Form III is at least 95%, such as at least 98%, after 30 days of storage at room temperature. In some embodiments, the chemical purity of polymorph Form III is at least 95%, such as at least 98%, after 120 days of storage at room temperature. In some embodiments, polymorph Form III is stable at elevated temperatures and/or relative humidity (RH). For example, polymorph Form III may be stored at about 40° C. and about 75% RH for an extended period of time-such as at least 10 days, at least 30 days, at least 60 days, at least 90 days, or at least 120 days-without significant chemical degradation or change in crystalline form. In some embodiments, the chemical purity of polymorph Form III is at least 95%, such as at least 98%, after 30 days of storage at 40° C. and 75% RH. In some embodiments, the chemical purity of polymorph Form III is at least 95%, such as at least 98%, after 120 days of storage at 40° C. and 75% RH. In some embodiments, polymorph Form III may be stored at about 60° C. for an extended period of time-such as at least 10 days, at least 30 days, at least 60 days, at least 90 days, or at least 120 days-without significant chemical degradation or change in crystalline form. In some embodiments, the chemical purity of polymorph Form III is at least 95%, such as at least 98%, after 30 days of storage at 60° C. In some embodiments, the chemical purity of polymorph Form III is at least 95%, such as at least 98%, after 120 days of storage at 60° C.

In certain aspects, the present disclosure provides a composition comprising a crystalline form of a compound of Formula I:

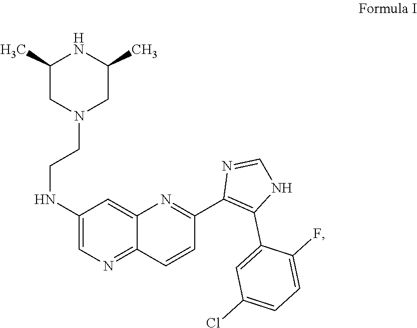 [SMILES: C[C@@H]1CN(CCNc2cnc3ccc(-c4nc[nH]c4-c4cc(Cl)ccc4F)nc3c2)C[C@H](C)N1]

or a pharmaceutically acceptable salt or solvate thereof.

In some embodiments, the composition comprises a crystalline form of a fumarate salt of the compound of Formula I. In some embodiments, the crystalline form is polymorph Form I of the fumarate salt of the compound of Formula I. In some embodiments, at least 90%, such as at least 95%, at least 96%, at least 97%, at least 98%, or at least 99%, of the fumarate salt of the compound of Formula I in the composition is polymorph Form I. The ratio of polymorph Form I to all other polymorphs in the composition may be at least 1:1 w/w, such as at least 2:1, 3:1, 4:1, 5:1, 6:1, 7:1, 8:1, 9:1, or at least 10:1 w/w.

In some embodiments, the crystalline form is polymorph Form II of the fumarate salt of the compound of Formula I. In some embodiments, at least 90%, such as at least 95%, at least 96%, at least 97%, at least 98%, or at least 99%, of the fumarate salt of the compound of Formula I in the composition is polymorph Form II. The ratio of polymorph Form II to all other polymorphs in the composition may be at least 1:1 w/w, such as at least 2:1, 3:1, 4:1, 5:1, 6:1, 7:1, 8:1, 9:1, or at least 10:1 w/w.

In some embodiments, the composition comprises a crystalline form of a freebase of the compound of Formula I. In some embodiments, the crystalline form is polymorph Form III of the freebase of the compound of Formula I. In some embodiments, at least 90%, such as at least 95%, at least 96%, at least 97%, at least 98%, or at least 99%, of the compound of Formula I in the composition is polymorph Form III. The ratio of polymorph Form III to all other polymorphs in the composition may be at least 1:1 w/w, such as at least 2:1, 3:1, 4:1, 5:1, 6:1, 7:1, 8:1, 9:1, or at least 10:1 w/w.

In certain aspects, the present disclosure provides a composition comprising one or more crystalline forms of the compound of Formula I, or a pharmaceutically acceptable salt or solvate thereof. In some embodiments, the composition comprises one or more crystalline forms of a fumarate salt or freebase of the compound of Formula I. The one or more crystalline forms may be selected from polymorph Form I, polymorph Form II and polymorph Form III.

In some embodiments, the composition can be stored at about 40° C. and 75% relative humidity for at least 30 days without significant degradation or change in the crystalline form. In some embodiments, the composition can be stored at about 60° C. and 75% relative humidity for at least 30 days without significant degradation or change in the crystalline form.

Conjugates

In certain aspects, the present disclosure provides a conjugate comprising the compound of Formula I, e.g., covalently linked, either directly or through a linker to an antibody construct or targeting moiety, thereby forming a conjugate. The linker may be a non-cleavable linker or a cleavable linker. A conjugate may be represented by the formula:

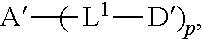

wherein A′ is an antibody construct or targeting moiety; L¹ is a linker; D′ is the compound of Formula I, or a salt thereof, and p is an integer from 1 to 20. In some embodiments, p is an integer from 1 to 10, such as from 1 to 8, 2 to 8, 1 to 6, 3 to 5, or from 1 to 3.

In some embodiments, a conjugate is represented by the formula:

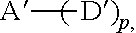

wherein A′ is an antibody construct or targeting moiety; D′ is the compound of Formula I, or a salt thereof, and p is an integer from 1 to 20. In some embodiments, p is an integer from 1 to 10, such as from 1 to 8, 2 to 8, 1 to 6, 3 to 5, or from 1 to 3.

Accordingly, the compound of Formula I, or a salt thereof, may be attached to A′ via a linker, L¹, or directly attached to A′ without an intermediate linker. In some embodiments, the compound or salt is covalently attached to an A′ or L¹. It will be understood by the skilled person that the compound of Formula I may be modified to introduce a suitable attachment site to A′ or L¹.

In some embodiments, L¹ or D′ is bound to A′ via a terminus of an amino acid sequence or via a side chain of an amino acid, such as the side chain of lysine, serine, threonine, cysteine, tyrosine, aspartic acid, glutamine, a non-natural amino acid residue, or glutamic acid residue. In some embodiments, L¹ or D′ is bound to A′ via one or more glycans or short peptide tags of four to six amino acids. L¹ or D′ may be conjugated to A′ via any suitable functional group, such as a thiol, an amine, an amide, an alcohol, a ketone, a carboxylic acid, or an ester.

A linker may be attached to a compound or salt of the present disclosure at any available position. For example, linker L¹ may be attached via a nitrogen atom of the compound of Formula I:

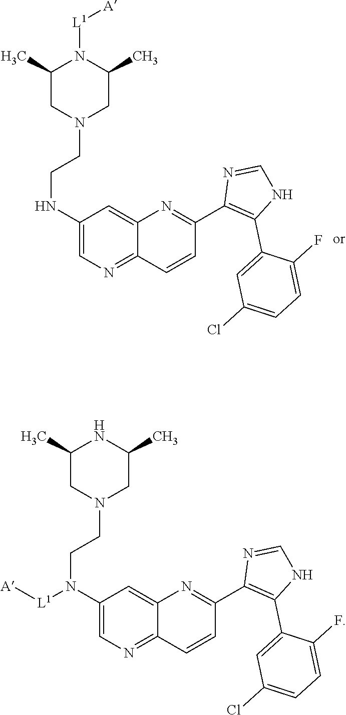

The compound of Formula I is typically depicted herein in its unconjugated form, though it will be understood by the skilled person that linker L¹ may be covalently bound to any suitable atom for attachment, such as a substitutable nitrogen or carbon of the compound. L¹ may be a cleavable or non-cleavable linker. The linker may further be bound to A′. Preferably, L¹ does not affect the binding of the active portions of the conjugate to the binding target(s). Covalent linkages may be formed by reaction between a functional group on the linker with a functional group on the compound, and by reaction between a functional group on the linker with a functional group on A′. As used herein in the context of conjugates, the term “linker” includes (i) unattached forms of the linker comprising a functional group capable of covalently attaching the linker to the compound of Formula I and a functional group capable of covalently attaching the linker to an antibody construct or targeting moiety; (ii) partially attached forms of the linker bound to the compound of Formula I, wherein the linker comprises a functional group capable of covalently attaching the linker to an antibody construct or targeting moiety; (iii) partially attached forms of the linker bound to an antibody construct or targeting moiety, wherein the linker comprises a functional group capable of covalently attaching the linker to the compound of Formula I; and (iv) fully attached forms of the linker bound to both an antibody construct or targeting moiety and the compound of Formula I.

Linker L¹ may be short, flexible, rigid, cleavable (e.g., by a lysosomal enzyme), non-cleavable, hydrophilic, or hydrophobic. A linker may contain segments having different characteristics, such as flexible segments and rigid segments. A linker may be chemically stable to extracellular environments, for example, in the bloodstream, or may include moieties that are not stable or are selectively stable. In some embodiments, a linker comprises a moiety that is selectively cleaved, for example, selectively cleaved in cells, a particular organ, or in plasma. A linker may be sensitive to enzymes, such as proteases. A linker may be insensitive to intracellular processes or proteases. A linker may be acid-labile, protease-sensitive or photolabile. In some embodiments, a linker comprises a peptide, succinimide, maleimide, polyethylene glycol, alkylene, alkenylene, alkynylene, disulfide, hydrazone, polyether, polyester, polyamide, aminobenzyl-carbamate, or a combination thereof.

In some aspects, the present disclosure provides the compound of Formula I, wherein the compound is covalently bound to A′, optionally via linker L¹. In some embodiments, the antibody construct is an antibody. In some embodiments, the present disclosure provides the compound of Formula I, wherein the compound is covalently bound to a linker, L¹, to form a compound-linker. A′ or L¹ may be covalently attached to any position of the compound, valence permitting. A linker L¹ disclosed herein may comprise from about 10 to about 500 atoms, such as 10 to 400 atoms, 10 to 300 atoms, 30 to 400 atoms, or 30 to 300 atoms.

The targets of the antibody, antibody construct, or targeting moiety may depend on the desired therapeutic applications of the conjugate. Typically, the targets are molecules present on the surfaces of cells into which it is desirable to deliver an ALK5 inhibitor, such as T cells, and the antibodies preferably internalize upon binding to the target. For applications in which the conjugates are intended to stimulate the immune system by reducing TGF-β activity, it may be desirable to generate antibodies, antibody constructs, or targeting moieties that bind to T cell surface molecules. Not wishing to be bound by any particular theory, it is believed that the delivery of ALK5 inhibitors to T cells can activate CD4⁺ and/or CD8⁺ T cell activity and inhibit regulatory T cell activity, both of which contribute to immune tolerance of tumors. Accordingly, antibodies, antibody constructs, or targeting moieties (A′) that bind to T cell surface molecules in the conjugates of the present disclosure are useful for the treatment of various cancers, such as those described herein below. In some embodiments, A′ binds to CD4⁺ T cells, CD8⁺ T cells, T_{REG} cells, or any combination thereof. In some embodiments, A′ binds to a pan T cell surface molecule, such as CD1, CD2, CD3, CD4, CD5, CD6, CD7, CD8, CD25, CD28, CD70, CD71, CD103, CD184, Tim3, LAG3, CTLA4, or PD1. Examples of antibodies that bind to T cell surface molecules and are believed to be internalizing include OKT6, OKT11, OKT3, OKT4, OKT8, 7D4, OKT9, CD28.2, UCHT1, M290, FR70, pembrolizumab, nivolumab, cemiplimab, and dostarlimab.

An antibody, antibody construct, or targeting moiety disclosed herein may comprise an antigen binding domain that specifically binds to a tumor antigen or antigen associated with the pathogenesis of fibrosis. In some embodiments, the antigen binding domain specifically binds to an antigen on a T cell, a B cell, a stellate cell, an endothelial cell, a tumor cell, an APC, a fibroblast cell, a fibrocyte cell, or a cell associated with the pathogenesis of fibrosis. In some embodiments, the antigen binding domain targets CTLA4, PD-1, OX40, LAG-3, GITR, GARP, CD25, CD27, PD-L1, TNFR2, ICOS, 41BB, CD70, CD73, CD38 or VTCN1. In some embodiments, the antigen binding domain targets PDGFRβ, integrin αvβ1, integrin αvβ3, integrin αvβ6, αvβ8, endosialin, FAP, ADAM12, LRRC15, MMP14, PDPN, CDH11, F2RL2, ASGR1, or ASGR2.

Methods

In some aspects, the present disclosure provides a method of inhibiting TGFβ signaling, comprising contacting a cell with an effective amount of a crystalline form disclosed herein. In some embodiments, the present disclosure provides a method of inhibiting ALK5, comprising contacting ALK5 with an effective amount of a crystalline form disclosed herein. Inhibition of ALK5 or TGFβ signaling can be assessed by a variety of methods known in the art. Non-limiting examples include a showing of (a) a decrease in kinase activity of ALK5; (b) a decrease in binding affinity between the TGFβ/TGFβ-RII complex and ALK5; (c) a decrease in the levels of phosphorylated intracellular signaling molecules downstream in the TGFβ signaling pathway, such as a decrease in pSMAD2 or pSMAD3 levels; (d) a decrease in binding of ALK5 to downstream signaling molecules, such as SMAD2 and SMAD3; and/or (e) an increase in ATP levels or a decrease in ADP levels. Kits and commercially available assays can be utilized for determining one or more of the above.

In some aspects, the present disclosure provides a method of treating an ALK5-mediated disease or condition in a subject, comprising administering to the subject a therapeutically effective amount of a crystalline form disclosed herein. In some embodiments, the disease or condition is selected from fibrosis and cancer. In some embodiments, the disease or condition is pulmonary fibrosis, such as idiopathic pulmonary fibrosis or virus-induced fibrosis. In some embodiments, the disease or condition is intestinal fibrosis. In some embodiments, the disease or condition is alopecia. In some embodiments, the disease is a neurodegenerative disease, such as Alzheimer's disease. In some embodiments, the present disclosure provides a method of reversing symptoms of aging. For example, the method may enhance neurogenesis, reduce neuroinflammation, improve cognitive performance, regenerate liver tissue, and/or reduce p16 levels.

In some aspects, the present disclosure provides a method of treating fibrosis, comprising administering to a patient an effective amount of a crystalline form disclosed herein. In some embodiments, the fibrosis is mediated by ALK5. In some embodiments, the fibrosis is selected from systemic sclerosis, systemic fibrosis, organ-specific fibrosis, kidney fibrosis, pulmonary fibrosis, liver fibrosis, portal vein fibrosis, skin fibrosis, bladder fibrosis, intestinal fibrosis, peritoneal fibrosis, myelofibrosis, oral submucous fibrosis, and retinal fibrosis. In some embodiments, the fibrosis is pulmonary fibrosis, such as idiopathic pulmonary fibrosis (IPF), familial pulmonary fibrosis (FPF), interstitial lung fibrosis, fibrosis associated with asthma, fibrosis associated with chronic obstructive pulmonary disease (COPD), silica-induced fibrosis, asbestos-induced fibrosis or chemotherapy-induced lung fibrosis. In some embodiments, the fibrosis is idiopathic pulmonary fibrosis (IPF). In some embodiments, the fibrosis is TGF-β-mediated pulmonary fibrosis. In some embodiments, the patient has been diagnosed with acute respiratory distress syndrome (ARDS). In some embodiments, the fibrosis is acute fibrosis. In some embodiments, the fibrosis is chronic fibrosis.

In some aspects, the present disclosure provides a method of treating pulmonary fibrosis induced by a viral infection, comprising administering to a patient an effective amount of a crystalline form disclosed herein. The pulmonary fibrosis may be induced by an erythrovirus, a dependovirus, a papillomavirus, a polyomavirus, a mastadenovirus, an alphaherpesvirinae, a varicellovirus, a gammaherpesvirinae, a betaherpesvirinae, a roseolovirus, an orthopoxvirus, a parapoxvirus, a molluscipoxvirus, an orthohepadnavirus, an enterovirus, a rhinovirus, a hepatovirus, an aphthovirus, a calicivirus, an astrovirus, an alphavirus, a rubivirus, a flavivirus, a Hepatitis C virus, a reovirus, an orbivirus, a rotavirus, an influenzavirus A, an influenzavirus B, an influenzavirus C, a paramyxovirus, a morbillivirus, a rubulavirus, a pneumovirus, a vesiculovirus, a lyssavirus, a bunyavirus, a hantavirus, a nairovirus, a phlebovirus, a coronavirus, an arenavirus, a BLV-HTLV-retrovirus, a lentivirinae, a spumavirnae or a filovirus. In some embodiments, the fibrosis is virus-induced fibrosis, such as virus-induced pulmonary fibrosis. In some embodiments, the fibrosis is selected from EBV-induced pulmonary fibrosis, CMV-induced pulmonary fibrosis, herpesvirus-induced pulmonary fibrosis and coronavirus-induced pulmonary fibrosis. In some embodiments, the fibrosis is selected from EBV-induced pulmonary fibrosis, CMV-induced pulmonary fibrosis, HHV-6-induced pulmonary fibrosis, HHV-7-induced pulmonary fibrosis, HHV-8-induced pulmonary fibrosis, H5N1 virus-induced pulmonary fibrosis, SARS-CoV-induced pulmonary fibrosis, MERS-CoV-induced pulmonary fibrosis and SARS-CoV-2-induced pulmonary fibrosis. In some embodiments, the pulmonary fibrosis is coronavirus-induced pulmonary fibrosis. In some embodiments, the pulmonary fibrosis is SARS-CoV-2-induced pulmonary fibrosis. In some embodiments, the pulmonary fibrosis is COVID-19-induced pulmonary fibrosis.

In some aspects, the present disclosure provides a method of treating acute lung injury (ALI), comprising administering to a patient an effective amount of a crystalline form disclosed herein. In some embodiments, the present disclosure provides a method of treating acute respiratory distress syndrome (ARDS), comprising administering to a patient an effective amount of a crystalline form disclosed herein. The ARDS may be in the early acute injury phase or the fibroproliferative phase. In some embodiments, the ARDS is fibroproliferative ARDS. In some embodiments, the present disclosure provides a method of treating fibrosis resulting from ARDS, comprising administering to a patient an effective amount of a crystalline form disclosed herein. The fibrosis resulting from ARDS may be pulmonary fibrosis. In some embodiments, the present disclosure provides a method of treating fibrosis resulting from ALI, comprising administering to a patient an effective amount of a crystalline form disclosed herein. The fibrosis resulting from ALI may be pulmonary fibrosis.

In some aspects, the present disclosure provides a method of treating intestinal fibrosis, comprising administering to a patient an effective amount of a crystalline form disclosed herein. In some embodiments, the intestinal fibrosis is mediated by ALK5. In some embodiments, the crystalline form is administered in an amount effective to delay progression of, reduce the incidence of, or reduce the degree of one or more characteristics associated with intestinal fibrosis. In some embodiments, the crystalline form is administered, either in a single dose or over multiple doses, in an amount effective to reverse established fibrosis.

In some aspects, the present disclosure provides a method of treating cancer, comprising administering to a patient an effective amount of a crystalline form disclosed herein. In some embodiments, the cancer is mediated by ALK5. In some embodiments, the cancer is selected from breast cancer, colon cancer, prostate cancer, lung cancer, hepatocellular carcinoma, glioblastoma, melanoma and pancreatic cancer. In some embodiments, the cancer is lung cancer, such as non-small cell lung cancer. In some aspects, the present disclosure provides a method of treating cancer, such as non-small cell lung cancer, comprising administering to a patient an effective amount of a crystalline form disclosed herein and an immunotherapeutic agent. In some embodiments, the cancer is stage III non-small cell lung cancer. In some embodiments, the method further comprises administering radiation to the patient. In some embodiments, the immunotherapeutic agent is a PD-1 inhibitor or a CTLA-4 inhibitor. In some embodiments, the immunotherapeutic agent is selected from atezolizumab, avelumab, nivolumab, pembrolizumab, durvalumab, BGB-A317, tremelimumab and ipilimumab. In some embodiments, the immunotherapeutic agent is selected from pembrolizumab and durvalumab.

The crystalline forms described herein, including polymorph Form I, polymorph Form II and polymorph Form III, are ALK5 inhibitors that limit the activity of TGFβ. TGFβ is one of several factors involved in the initiation and development of fibrotic diseases throughout the body. As such, the crystalline forms of the disclosure are expected to be useful for the treatment, prevention and/or reduction of fibrosis in a patient by administering a therapeutically effective amount of a crystalline form disclosed herein. By inhibiting ALK5, the crystalline form is expected to potentiate the formation of fibrosis in areas of the body that suffer from excessive deposition of the extracellular matrix. Those areas are described below.

Systemic Fibrotic Diseases

Systemic sclerosis (SSc) is an autoimmune disorder that affects the skin and internal organs and results in autoantibody production, vascular endothelial activation of small blood vessels, and tissue fibrosis as a result of fibroblast dysfunction. Transforming growth factor β (TGF-β) has been identified as a regulator of pathological fibrogenesis in SSc (Ayers, N. B., et al., Journal of Biomedical Research, 2018, 32(1), pp. 3-12). According to the authors, “understanding the essential role TGF-β pathways play in the pathology of systemic sclerosis could provide a potential outlet for treatment and a better understanding of this severe disease.” In some embodiments, the present disclosure provides a method of treating SSc, comprising administering to a subject an effective amount of a crystalline form disclosed herein.

Multifocal fibrosclerosis (MF) and idiopathic multifocal fibrosclerosis (IMF) are disorders characterized by fibrous lesions at varying sites and include retroperitoneal fibrosis, mediastinal fibrosis and Riedel's thyroiditis. Both multifocal fibrosclerosis and idiopathic multifocal fibrosclerosis are considered to be an outcome of IgG₄-associated fibrosis/disease and TGF-β is believed to be one factor involved in the initiation and development of fibrosis (Pardali, E., et. al., Int. J. Mol. Sci., 18, 2157, pp. 1-22). In some embodiments, the present disclosure provides a method of treating multifocal fibrosclerosis or idiopathic multifocal fibrosclerosis, comprising administering to a subject an effective amount of a crystalline form disclosed herein.

In some embodiments, the present disclosure provides a method of treating nephrogenic systemic fibrosis, comprising administering to a subject an effective amount of a crystalline form disclosed herein. Nephrogenic systemic fibrosis is a rare disease occurring mainly in people with advanced kidney failure with or without dialysis. In a study performed by Kelly et al. (J. Am. Acad. Dermatol., 2008, 58, 6, pp. 1025-1030), it was shown that TGF-β, as well as Smad 2/3, appear to be associated with fibrosis seen in nephrogenic systemic fibrosis.

Sclerodermatous graft-versus-host disease (GVHD) is a prevalent complication of allogeneic hematopoietic stem cell graft appearing two to three months after allogeneic bone marrow transplantation. The disease results in production of autoantibodies and fibrosis of skin and inner organs. Using a murine cutaneous GVHD model, it has been shown that progression of early skin and lung disease can be inhibited with TGF-β neutralizing antibodies (McCormick, L. L., et al., J. Immunol., 1999, 163, pp. 5693-5699). In some embodiments, the present disclosure provides a method of treating sclerodermatous GVHD, comprising administering to a subject an effective amount of a crystalline form disclosed herein.

Organ-Specific Fibrotic Diseases

Cardiac fibrosis refers to the abnormal thickening of heart valves due to the abnormal proliferation of cardiac fibroblasts resulting in excess deposition of ECM in heart muscle. Fibroblasts secrete collagen, which serves as structural support for the heart. However, when collagen is excessively secreted in the heart, wall and valve thickening can result in tissue build-up on the tricuspid and pulmonary valves. This in turn causes loss of flexibility and ultimately valvular dysfunction leading to heart failure. A specific type of cardiac fibrosis is hypertension-associated cardiac fibrosis as described by J. Diez (J. Clin. Hypertens., 2007, July 9(7), pp. 546-550). According to Diez, changes in the composition of cardiac tissue develop in hypertensive patients with left ventricular hypertrophy and lead to structural remodeling of the heart tissue. One change relates to the disruption of the equilibrium between the synthesis and degradation of collagen types I and III molecules, resulting in excessive accumulation of collagen fibers in the heart tissue. Other types of cardiac fibrosis include post-myocardial infarction and Chagas disease-induced myocardial fibrosis. In Chagas disease, transforming growth factor β1 (TGF-β1) has been implicated in Chagas disease physiopathology, where animal models suggest that the TGF-β1-pathway is up-regulated during infection (Araujo-Jorge, T. C., et al., Clin. Pharmacol. Ther., 2012, 92(5), pp. 613-621; Curvo, E., Mem Inst Oswaldo Cruz, 2018, Vol. 113(4), e170440, pp. 1-8). In some embodiments, the present disclosure provides a method of treating various forms of cardiac fibrosis, such as hypertension-associated cardiac fibrosis, post-myocardial infarction or Chagas disease-induced myocardial fibrosis, comprising administering to a subject an effective amount of a crystalline form disclosed herein.

Renal fibrosis encompasses a variety of disorders associated with the aberrant expression and activity of TGF-β, including, but not limited to, diabetic and hypertensive nephropathy, urinary tract obstruction-induced kidney fibrosis, inflammatory/autoimmune-induced kidney fibrosis, aristolochic acid nephropathy, progressive kidney fibrosis, and polysystic kidney disease. As discussed above, fibrosis involves an excess accumulation of the ECM, which in turn causes loss of function when normal tissue is replaced with scar tissue (Wynn, T. A., J Clin Invest., 2007, 117, pp. 524-529). As early as 2005, ALK5 inhibitors were being studied in models for renal disease (Laping, N. J., Current Opinion in Pharmacology, 2003, 3, pp. 204-208). In some embodiments, the present disclosure provides a method of treating renal fibrosis, comprising administering to a subject an effective amount of a crystalline form disclosed herein.

One fibrotic disease that has been particularly difficult to treat is idiopathic pulmonary fibrosis (IPF). IPF is a chronic, progressive and fatal fibrotic lung disease with survival only improved by lung transplantation. Current oral therapies such as nintedanib and pirfenidone have been shown to slow the progression of the disease, but have adverse effects that lead to discontinuation and lack of compliance by the patient. Although there are other therapies in development targeting various pathways, an unmet need remains for patients with IPF.

Although ALK5 is an important and known component in the fibrotic disease pathway, the efficacy of ALK5 inhibitors in IPF have not been realized due to systemic adverse effects, especially in the heart. Thus, one of the goals of this disclosure is to develop ALK5 inhibitors with high lung selectivity and rapid systemic clearance. One preferred embodiment of this disclosure is to treat patients with idiopathic pulmonary fibrosis with a crystalline form described herein, for example, by once or twice daily administration of inhalable ALK5 inhibitor having minimal systemic exposure. The inhaled ALK5 inhibitor may be administered as a monotherapy or co-dosed with other orally available IPF therapies. In some embodiments, the present disclosure provides a method of treating idiopathic pulmonary fibrosis, comprising administering to a subject an effective amount of a crystalline form disclosed herein. In some embodiments, the crystalline form is administered by inhalation.

Familial pulmonary fibrosis is a hereditary disease where two or more family members have confirmed IPF. In some embodiments, the present disclosure provides a method of treating familial pulmonary fibrosis, comprising administering to a subject an effective amount of a crystalline form disclosed herein.

Pulmonary fibrosis is a typical clinical feature associated with viral infection, such as SARS and COVID-19. SARS-mediated TGF-β signaling has been shown to promote fibrosis and block apoptosis of SARS-CoV-infected host cells (Zhao, X. et al., J Biol. Chem., 2008, 283(6), pp. 3272-3280). Increased TGF-β expression was similarly observed in patients infected with SARS-CoV-2, ultimately leading to the development of pulmonary fibrosis. TGF-β signaling mediated by SARS-CoV-2 can promote fibroblast proliferation and myofibroblast differentiation and block host cell apoptosis. (Xiong, Y. et al., Emerging Microbes & Infections, 2020, 9(1), pp. 761-770). Crystalline forms of the present disclosure are expected to inhibit increased TGF-β signaling mediated by viral infection and prevent, halt, slow or reverse the progression of pulmonary fibrosis associated with the infection. Accordingly, in some embodiments, the present disclosure provides a method of treating pulmonary fibrosis induced by a viral infection, comprising administering to a subject an effective amount of a crystalline form disclosed herein. In some embodiments, the pulmonary fibrosis is induced by SARS-CoV or SARS-CoV-2. In some embodiments, the crystalline form is administered by inhalation.

Chronic lung disease, such as interstitial lung disease (ILD), chronic obstructive pulmonary disease (COPD) and idiopathic pulmonary fibrosis (IPF), may lead to pulmonary hypertension (PH). Pulmonary hypertension is a progressive disease characterized by high blood pressure in the lungs. The World Health Organization (WHO) has defined five classifications of PH (WHO Group I: Pulmonary arterial hypertension (PAH); WHO Group II: Pulmonary hypertension due to left heart disease; WHO Group III: Pulmonary hypertension due to lung disease and/or hypoxia; WHO Group IV: Chronic thromboembolic pulmonary hypertension (CTEPH); and WHO Group V: Pulmonary hypertension with unclear multifactorial mechanisms). TGF-β signaling has been implicated in the pathogenesis of PH. Moreover, inhibition of ALK5 in a monocrotaline (MCT) model of severe PH was shown to attenuate the development of PH and reduce pulmonary vascular remodeling in a dose-dependent manner, namely by reducing RV systolic pressure, reducing RV diastolic pressure, increasing cardiac output and reducing RV hypertrophy (Zaiman, A. L.; et al., Am. J. Respir. Crit. Care Med., 2008, 177, pp. 896-905). Crystalline forms of the present disclosure are expected to inhibit TGF-β signaling in lung tissue and prevent, halt, slow or reverse the progression of PH, particularly in WHO Group III PH. Accordingly, in some embodiments, the present disclosure provides a method of treating pulmonary hypertension, comprising administering to a subject an effective amount of a crystalline form disclosed herein. The pulmonary hypertension may be WHO Group III pulmonary hypertension, such as pulmonary fibrosis-related pulmonary hypertension (PH-PF) or interstitial lung disease-related pulmonary hypertension (PH-ILD). In some embodiments, the crystalline form is administered by inhalation.

Other types of interstitial lung diseases include, but are not limited to, (1) interstitial pneumonia caused by bacteria, viruses, or fungi; (2) nonspecific interstitial pneumonitis usually associated with autoimmune conditions such as rheumatoid arthritis or scleroderma; (3) hypersensitivity pneumonitis caused by inhalation of dust, mold, or other irritants; (4) cryptogenic organizing pneumonia; (5) acute interstitial pneumonitis; (6) desquamative interstitial pneumonitis; (7) sarcoidosis; and (8) drug-induced interstitial lung disease. In some embodiments, the present disclosure provides a method of treating an interstitial lung disease, comprising administering to a subject an effective amount of a crystalline form disclosed herein.

Both transforming growth factor (TGF)-beta(1) and activin-A have been implicated in airway remodeling in asthma (Kariyawasam, H. H., J Allergy Clin Immunol., 2009, September, 124(3), pp. 454-462). In some embodiments, the present disclosure provides a method of treating asthma, comprising administering to a subject an effective amount of a crystalline form disclosed herein.

Chronic obstructive pulmonary disease (COPD) is a pulmonary disorder characterized by a poorly reversible and progressive airflow limitation caused by airway inflammation and emphysema, whereas IPF is associated with impaired diffusion capacity (Chilosi, M., et al., Respir. Res., 2012, 13(1), 3, pp. 1-9). Both diseases, however, demonstrate a progressive loss of alveolar parenchyma leading to severe impairment of respiratory function. Fibrosis associated with emphysema is known and research has demonstrated TGF-β1 involvement in chronic sinus disease, pulmonary fibrosis, asthma, and COPD (Yang, Y. C., et al., Allergy, 2012, 67, pp. 1193-1202). In some embodiments, the present disclosure provides a method of treating COPD, comprising administering to a subject an effective amount of a crystalline form disclosed herein.

Other types of lung injury that result in fibrosis include silica-induced pneumoconiosis (silicosis), asbestos-induced pulmonary fibrosis (asbestosis), and chemotherapeutic agent-induced pulmonary fibrosis. In some embodiments, the present disclosure provides a method of treating injury-induced fibrosis, comprising administering to a subject an effective amount of a crystalline form disclosed herein.

In some embodiments, the present disclosure provides a method of treating liver fibrosis, comprising administering to a subject an effective amount of a crystalline form disclosed herein. Fibrosis develops in the liver when it is repeatedly or continuously damaged, for example, in patients with chronic hepatitis. TGF-β signaling participates in all stages of disease progression, from initial liver injury through inflammation and fibrosis, to cirrhosis and cancer (Fabregat, I., et al., The FEBS J., 2016, 283(12), pp. 2219-2232).

A related condition involves fibrosis resulting from idiopathic non-cirrhotic portal hypertension (INCPH). This disease is of uncertain etiology characterized by periportal fibrosis and involvement of small and medium branches of the portal vein. According to Nakanuma et al., small portal veins and skin findings are similar between patients with scleroderma and INCPH (Nakanuma, Y., Hepatol. Res., 2009, 39, pp. 1023-1031). Transforming growth factor-β (TGF-β) and connective tissue growth factor, which are fibrosis-related and vascular endothelial growth factors, respectively, increase in serum, skin, and the portal vein, suggesting that these could be mechanisms of the portal vein occlusion in INCPH. Moreover, endothelial mesenchymal transition (EndMT) theory was proposed by Kitao et al. based on these findings (Kitao, A., et al., Am. J. Pathol., 2009, 175, pp. 616-626). The increase of TGF-β in sera may act as a potent inducer of EndMT. In some embodiments, the present disclosure provides a method of treating INCPH, comprising administering to a subject an effective amount of a crystalline form disclosed herein.

Other types of liver fibrosis include alcoholic and non-alcoholic liver fibrosis, hepatitis C-induced liver fibrosis, primary biliary cirrhosis or cholangitis, and parasite-induced liver fibrosis (schistosomiasis). In some embodiments, the present disclosure provides a method of treating alcoholic liver fibrosis, non-alcoholic liver fibrosis, hepatitis C-induced liver fibrosis, primary biliary cirrhosis, primary biliary cholangitis, or parasite-induced liver fibrosis (schistosomiasis), comprising administering to a subject an effective amount of a crystalline form disclosed herein.

Primary biliary cholangitis (PBC) and primary sclerosing cholangitis (PSC) are two types of chronic liver disease that often lead to cirrhosis and liver failure. Liver biopsies of patients with PBC or PSC typically reveal inflammation and fibrosis. Inhibition of integrin αvβ6, which has been shown to bind to and activate TGFβ1 on epithelial cells, suppresses biliary fibrosis in rodents. (Peng, Z-W., et al., Hepatology, 2016, 63, pp. 217-232). Accordingly, inhibition of the TGF-β pathway is also expected to suppress fibrotic processes in both PBC and PSC. Crystalline forms of the present disclosure are expected to inhibit TGF-β signaling in liver tissue and prevent, halt, slow or reverse the progression of PBC and PSC. Thus, in some embodiments, the present disclosure provides a method of treating primary biliary cholangitis or primary sclerosing cholangitis, comprising administering to a subject an effect amount of a crystalline form described herein. In some embodiments, the present disclosure provides a method of treating liver fibrosis, optionally in a subject that suffers from PBC or PSC, comprising administering to the subject an effective amount of a crystalline form described herein.

Fibrotic skin conditions include, but are not limited to, hypertrophic scarring, keloids, and localized or systemic sclerosis (scleroderma). As discussed previously, TGF-β is a potent stimulus of connective tissue accumulation and has been implicated in the pathogenesis of scleroderma and other fibrotic disorders (Lakos, G., et al., Am. J. Pathol., 2004, 165(1), pp. 203-217). Lakos et. al. demonstrated that Smad3 functions as a key intracellular signal transducer for profibrotic TGF-β responses in normal skin fibroblasts and found that the targeted disruption of TGF-β/Smad3 signaling modulated skin fibrosis in the mouse model of scleroderma. In some embodiments, the present disclosure provides a method of treating skin fibrosis, comprising administering to a subject an effective amount of a crystalline form disclosed herein.

Intestinal fibrosis is a common complication of inflammatory bowel disease (IBD) and is a serious clinical problem. TGF-β has been implicated as a major driving factor of intestinal fibrosis. Moreover, TGF-β1 signaling contributes to stricture formation in fibrostenotic Crohn's disease by inducing insulin-like growth factor I (IGF-I) and mechano-growth factor (MGF) production in intestinal smooth muscle. (Latella, G., Rieder, F., Curr. Opin. Gastroenterol., 2017, 33(4), pp. 239-245). Inhibition of TGF-β signaling could thus slow, halt or reverse the progression of fibrosis in the intestine. However, adverse side effects of concern to patients with IBD—such as inflammation and neoplasia—would likely result from systemic inhibition of TGF-β signaling. One goal of the present disclosure is to develop ALK5 inhibitors with high selectivity for the gastrointestinal tract and rapid clearance. In some embodiments, the present disclosure provides a method of treating intestinal fibrosis, comprising administering to a subject an effective amount of a crystalline form described herein, for example, by once or twice daily administration of an oral ALK5 inhibitor having minimal systemic exposure. In some embodiments, the subject suffers from inflammatory bowel disease, such as Crohn's disease or colitis. The degree of therapeutic efficacy may be with respect to a starting condition of the subject (e.g., a baseline Mayo score, baseline Lichtiger score, or severity or incidence of one or more symptoms), or with respect to a reference population (e.g., an untreated population, or a population treated with a different agent). Severity of intestinal fibrosis may be assessed using any suitable method, such as delayed enhancement MRI, ultrasound elastography, shear wave elastography, magnetization MRI, or by the direct detection of macromolecules such as collagen. Preferably, treatment with a crystalline form of the present disclosure reduces the severity of the fibrosis, such as from severe fibrosis to moderate or mild fibrosis. In some embodiments, the treatment increases intestinal tissue elasticity, reduces tissue stiffness, and/or reduces collagen levels. In some embodiments, the treatment prevents myofibroblast accumulation, inhibits expression of pro-fibrotic factors, and/or inhibits accumulation of fibrotic tissue.

Other types of organ-specific fibrosis or fibrotic diseases involving the TGF-β pathway include, but are not limited to, radiation-induced fibrosis (various organs), bladder fibrosis, intestinal fibrosis, peritoneal sclerosis, diffuse fasciitis, Dupuytren's disease, myelofibrosis, oral submucous fibrosis, and retinal fibrosis. In some embodiments, the present disclosure provides a method of treating radiation-induced fibrosis, bladder fibrosis, intestinal fibrosis, peritoneal sclerosis, diffuse fasciitis, Dupuytren's disease, myelofibrosis, oral submucous fibrosis, or retinal fibrosis, comprising administering to a subject an effective amount of a crystalline form disclosed herein.

Although one of the goals of this disclosure is to treat fibrotic and pulmonary diseases locally or in a targeted way, the crystalline forms described herein may also be used to treat patients systemically. Diseases that may be treated systemically, include, for example, oncologic diseases such as glioblastoma, pancreatic cancer and hepatocellular carcinoma, breast cancer metastasized to lungs, non-small cell lung cancer, small cell lung cancer, cystic fibrosis, and metastasis of other forms of primary cancer subtypes. Some of the forgoing diseases may also be treated locally as well.

Other fibrotic diseases that crystalline forms disclosed herein may treat include angioedema, anti-aging, and alopecia. Alopecia includes alopecia totalis, alopecia universalis, androgenetic alopecia, alopecia areata, diffuse alopecia, postpartum alopecia, and traction alopecia.

Other Indications

In certain aspects, the present disclosure provides a method of reversing one or more symptoms of aging, comprising administering to a subject crystalline form disclosed herein. The method may further comprise administering an activator of the MAPK pathway, such as oxytocin. The method may be effective in one or more of enhancing neurogenesis in the hippocampus, reducing neuroinflammation, improving cognitive ability, reducing liver adiposity, reducing liver fibrosis, and decreasing the number of p16′ cells. In some embodiments, a method described herein increases stem cell activity. The increase in stem cell activity may allow the subject to generate new muscle fibers and/or to form new neurons in the hippocampus. Treatment with an ALK5 inhibitor, such as a crystalline form described herein, may prevent or slow the onset of age-related diseases, such as Alzheimer's disease. (see Mehdipour, M. et al. Aging 2018, 10, 5628-5645).

In practicing any of the subject methods, a crystalline form may be administered directly to a subject, for example, as a solid or suspension, or the crystalline form may be used to prepare a composition, such as a solution, that is administered to the subject. Thus, references to administering to a subject a crystalline form include administering compositions prepared from the crystalline form of the compound of Formula I, whether or not the compound of Formula I is present in crystalline form or in solution when administered to the subject.

Pharmaceutical Compositions

In certain aspects, the present disclosure provides a pharmaceutical composition. The pharmaceutical composition may comprise a crystalline form disclosed herein, such as polymorph Form I, polymorph Form II or polymorph Form III, and a pharmaceutically acceptable carrier. The pharmaceutical composition may comprise a salt disclosed herein, such as a fumarate salt of the compound of Formula I, and a pharmaceutically acceptable carrier. In some embodiments, the pharmaceutical composition is formulated for oral administration. In some embodiments, the pharmaceutical composition is formulated for inhalation. In some embodiments, the pharmaceutical composition is prepared from the crystalline form, even though the compound of Formula I is in solution in the pharmaceutical composition. In some embodiments, the pharmaceutical composition comprises a crystalline form disclosed herein and an additional therapeutic agent. Non-limiting examples of such therapeutic agents are described herein below.

Pharmaceutical compositions typically include at least one pharmaceutically acceptable carrier, diluent or excipient and at least one crystalline form disclosed herein (also referred to herein as the active agent). The active agent may be provided in any form suitable for the particular mode of administration, such as a free base, a free acid, or a pharmaceutically acceptable salt. All tautomers of the compounds described herein are included within the scope of the present disclosure. Additionally, the compounds described herein encompass unsolvated as well as solvated forms with pharmaceutically acceptable solvents such as water, ethanol and the like.

Suitable routes of administration include, but are not limited to, oral, intravenous, rectal, vaginal, aerosol, pulmonary, nasal, transmucosal, topical, transdermal, otic, ocular, and parenteral modes of administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a crystalline form described herein is administered in a local rather than systemic manner, for example, via injection of the crystalline form directly into an organ, often in a depot preparation or sustained release formulation. In some embodiments, a long acting formulation is administered by implantation (e.g., subcutaneously or intramuscularly) or by intramuscular injection. In some embodiments, a crystalline form described herein is provided in the form of a rapid release formulation, an extended release formulation, or an intermediate release formulation. In some embodiments, a crystalline form described herein is provided in the form of a nebulized formulation. In some embodiments, a crystalline form described herein is administered locally to the lungs by inhalation.

Crystalline forms of the present disclosure are effective over a wide dosage range. For example, in the treatment of adult humans, dosages from 0.01 to 1000 mg, 0.5 to 100 mg, 1 to 50 mg, or from 5 to 40 mg per day may be administered to a subject in need thereof. The exact dosage will depend upon the route of administration, the form in which the crystalline form is administered, the subject to be treated, the body weight of the subject to be treated, and the preference and experience of the attending physician.

A crystalline form of the present disclosure may be administered in a single dose. In some embodiments, a crystalline form disclosed herein is administered in multiple doses, such as about once, twice, three times, four times, five times, six times, or more than six times per day. In some embodiments, dosing is about once a month, once every two weeks, once a week, or once every other day. In some embodiments, a crystalline form of the disclosure and an additional therapeutic agent are administered together about once per day to about 6 times per day. In some embodiments, the administration continues for more than about 6, 10, 14, 28 days, two months, six months, or more than about one year. In some embodiments, a dosing schedule is maintained as long as necessary. A crystalline form of the present disclosure may be administered chronically on an ongoing basis, e.g., for the treatment of chronic effects.

Pharmaceutical compositions of the present disclosure typically contain a therapeutically effective amount of a crystalline form of the present disclosure. Those skilled in the art will recognize, however, that a pharmaceutical composition may contain more than a therapeutically effective amount, e.g., bulk compositions, or less than a therapeutically effective amount, e.g., individual unit doses designed for co-administration to achieve a therapeutically effective amount.

Typically, pharmaceutical compositions of the present disclosure contain from about 0.01 to about 95% by weight of the active agent; including, for example, from about 0.05 to about 30% by weight; and from about 0.1% to about 10% by weight of the active agent.

Any conventional carrier or excipient may be used in the pharmaceutical compositions of the present disclosure. The choice of a particular carrier or excipient, or combinations of carriers or excipients, will depend on the mode of administration being used to treat a particular patient or type of medical condition or disease state. Additionally, the carriers or excipients used in the pharmaceutical compositions of this disclosure may be commercially-available. Conventional formulation techniques are described in Remington: The Science and Practice of Pharmacy, 20th Edition, Lippincott Williams & White, Baltimore, Md. (2000); and H. C. Ansel et al., Pharmaceutical Dosage Forms and Drug Delivery Systems, 7th Edition, Lippincott Williams & White, Baltimore, Md. (1999).

Representative examples of materials which can serve as pharmaceutically acceptable carriers include, but are not limited to, the following: sugars, such as lactose, glucose and sucrose; starches, such as corn starch and potato starch; cellulose, such as microcrystalline cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical compositions.

Pharmaceutical compositions are typically prepared by thoroughly and intimately mixing or blending the active agent with a pharmaceutically-acceptable carrier and one or more optional ingredients. The resulting uniformly blended mixture can then be shaped or loaded into tablets, capsules, pills and the like using conventional procedures and equipment.

In one aspect, the pharmaceutical composition is suitable for inhaled administration. Pharmaceutical compositions for inhaled administration are typically in the form of an aerosol or a powder. Such compositions are generally administered using inhaler delivery devices, such as a dry powder inhaler (DPI), a metered-dose inhaler (MDI), a nebulizer inhaler, or a similar delivery device.

In a particular embodiment, the pharmaceutical composition is administered by inhalation using a dry powder inhaler. Such dry powder inhalers typically administer the pharmaceutical composition as a free-flowing powder that is dispersed in a patient's air-stream during inspiration. In order to achieve a free-flowing powder composition, the therapeutic agent is typically formulated with a suitable excipient such as lactose, starch, mannitol, dextrose, polylactic acid (PLA), polylactide-co-glycolide (PLGA) or combinations thereof. Typically, the therapeutic agent is micronized and combined with a suitable carrier to form a composition suitable for inhalation.

A representative pharmaceutical composition for use in a dry powder inhaler comprises lactose and a micronized form of a crystalline form disclosed herein. Such a dry powder composition can be made, for example, by combining dry milled lactose with the therapeutic agent and then dry blending the components. The composition is then typically loaded into a dry powder dispenser, or into inhalation cartridges or capsules for use with a dry powder delivery device.

Dry powder inhaler delivery devices suitable for administering therapeutic agents by inhalation are described in the art and examples of such devices are commercially available. For example, representative dry powder inhaler delivery devices or products include Aeolizer (Novartis); Airmax (IVAX); ClickHaler (Innovata Biomed); Diskhaler (GlaxoSmithKline); Diskus/Accuhaler (GlaxoSmithKline); Ellipta (GlaxoSmithKline); Easyhaler (Orion Pharma); Eclipse (Aventis); FlowCaps (Hovione); Handihaler (Boehringer Ingelheim); Pulvinal (Chiesi); Rotahaler (GlaxoSmithKline); SkyeHaler/Certihaler (SkyePharma); Twisthaler (Schering-Plough); Turbuhaler (AstraZeneca); Ultrahaler (Aventis); and the like.

A pharmaceutical composition of the present disclosure may be administered by inhalation using a metered-dose inhaler. Such metered-dose inhalers typically discharge a measured amount of a therapeutic agent using a compressed propellant gas. Accordingly, pharmaceutical compositions administered using a metered-dose inhaler typically comprise a solution or suspension of the therapeutic agent in a liquefied propellant. Any suitable liquefied propellant may be employed, including hydrofluoroalkanes (HFAs), such as 1,1,1,2-tetrafluoroethane (HFA 134a) and 1,1,1,2,3,3,3-heptafluoro-n-propane, (HFA 227); and chlorofluorocarbons, such as CCl₃F. In a particular embodiment, the propellant is a hydrofluoroalkane. In some embodiments, the hydrofluoroalkane formulation contains a co-solvent, such as ethanol or pentane, and/or a surfactant, such as sorbitan trioleate, oleic acid, lecithin, and glycerin.

A representative pharmaceutical composition for use in a metered-dose inhaler comprises from about 0.010% to about 5% by weight of a crystalline form of the present disclosure; from about 0% to about 20% by weight ethanol; and from about 0% to about 5% by weight surfactant; with the remainder being an HFA propellant. Such compositions are typically prepared by adding chilled or pressurized hydrofluoroalkane to a suitable container containing the therapeutic agent, ethanol (if present) and the surfactant (if present). To prepare a suspension, the therapeutic agent is micronized and then combined with the propellant. The composition is then loaded into an aerosol canister, which typically forms a portion of a metered-dose inhaler device.

Metered-dose inhaler devices suitable for administering therapeutic agents by inhalation are described in the art and examples of such devices are commercially available. For example, representative metered-dose inhaler devices or products include AeroBid Inhaler System (Forest Pharmaceuticals); Atrovent Inhalation Aerosol (Boehringer Ingelheim); Flovent (GlaxoSmithKline); Maxair Inhaler (3M); Proventil Inhaler (Schering); Serevent Inhalation Aerosol (GlaxoSmithKline); and the like.

A pharmaceutical composition of the present disclosure may be administered by inhalation using a nebulizer inhaler. Such nebulizer devices typically produce a stream of high velocity air that causes the pharmaceutical composition to spray as a mist that is carried into the patient's respiratory tract. Accordingly, when formulated for use in a nebulizer inhaler, the crystalline form can be dissolved in a suitable carrier to form a solution of the compound of Formula I. Alternatively, the crystalline form can be micronized or nanomilled and combined with a suitable carrier to form a suspension.

A representative pharmaceutical composition for use in a nebulizer inhaler comprises a solution or suspension comprising from about 0.05 μg/mL to about 20 mg/mL of a crystalline form of the present disclosure and excipients compatible with nebulized formulations. In one embodiment, the solution has a pH of about 3 to about 8.

Nebulizer devices suitable for administering therapeutic agents by inhalation are described in the art and examples of such devices are commercially available. For example, representative nebulizer devices or products include the Respimat® Softmist™ Inhalaler (Boehringer Ingelheim); the AERx® Pulmonary Delivery System (Aradigm Corp.); the PARI LC Plus®Reusable Nebulizer or PARI eFlow®rapid Nebulizer System (Pari GmbH); and the like.

A pharmaceutical composition of the present disclosure may be prepared in a dosage form intended for oral administration. Suitable pharmaceutical compositions for oral administration may be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a crystalline form of the present disclosure as an active ingredient.

When intended for oral administration in a solid dosage form, the pharmaceutical compositions of the disclosure will typically comprise the active agent and one or more pharmaceutically-acceptable carriers, such as sodium citrate or dicalcium phosphate. Optionally or alternatively, such solid dosage forms may also comprise: fillers or extenders, binders, humectants, solution retarding agents, absorption accelerators, wetting agents, absorbents, lubricants, coloring agents, and buffering agents. Release agents, wetting agents, coating agents, sweetening, flavoring and perfuming agents, preservatives and antioxidants can also be present in the present pharmaceutical compositions.

Alternative formulations may include controlled release formulations, liquid dosage forms for oral administration, transdermal patches, and parenteral formulations. Conventional excipients and methods of preparation of such alternative formulations are described, for example, in the reference by Remington, supra.

The following non-limiting examples illustrate representative pharmaceutical compositions of the present disclosure.

Dry Powder Composition

A micronized crystalline form of the present disclosure (1 g) is blended with milled lactose (25 g). This blended mixture is then loaded into individual blisters of a peelable blister pack in an amount sufficient to provide between about 0.1 mg to about 4 mg of the crystalline form per dose. The contents of the blisters are administered using a dry powder inhaler.

Dry Powder Composition

A micronized crystalline form of the present disclosure (1 g) is blended with milled lactose (20 g) to form a bulk composition having a weight ratio of crystalline form to milled lactose of 1:20. The blended composition is packed into a dry powder inhalation device capable of delivering between about 0.1 mg to about 4 mg of the crystalline form per dose.

Metered-Dose Inhaler Composition

A micronized crystalline form of the present disclosure (10 g) is dispersed in a solution prepared by dissolving lecithin (0.2 g) in demineralized water (200 mL). The resulting suspension is spray dried and then micronized to form a micronized composition comprising particles having a mean diameter less than about 1.5 μm. The micronized composition is then loaded into metered-dose inhaler cartridges containing pressurized 1,1,1,2-tetrafluoroethane in an amount sufficient to provide about 0.1 mg to about 4 mg of the compound of Formula I per dose when administered by the metered dose inhaler.

Nebulizer Composition

A representative nebulizer composition is as follows. A crystalline form of the present disclosure (2 g of free-base equivalents) is dissolved in a solution containing 80 mL reverse-osmosis water, 0.1-1% by weight of anhydrous citric acid, and 0.5-1.5 equivalents of hydrochloric acid, followed by addition of sodium hydroxide to adjust the pH to 3.5 to 5.5. Thereafter, between 4-6% by weight of D-mannitol is added and solution q.s. to 100 mL. The solution is then filtered through a 0.2 μm filter and stored at room temperature prior to use. The solution is administered using a nebulizer device that provides about 0.1 mg to about 4 mg of the compound of Formula I per dose.

Kits

In certain aspects, the present disclosure provides a kit comprising one or more unit doses of a crystalline form or pharmaceutical composition described herein, optionally wherein the kit further comprises instructions for using the crystalline form or pharmaceutical composition. In some embodiments, the kit comprises a carrier, package, or container that is compartmentalized to receive one or more containers, such as vials, tubes, and the like, each of the container(s) comprising one of the separate elements to be used in a method described herein. Suitable containers include, for example, bottles, vials, syringes, and test tubes. The containers may be formed from a variety of materials, such as glass or plastic.

The articles of manufacture provided herein may contain packaging materials. Packaging materials for use in packaging pharmaceutical products include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) may include one or more crystalline forms described herein, optionally in a composition or in combination with another agent as disclosed herein. The container(s) may optionally have a sterile access port (for example, the container is an intravenous solution bag or a vial having a stopper pierceable by a hypodermic injection needle). Such kits may optionally comprise a crystalline form with an identifying description or label or instructions relating to its use in the methods described herein.

In some embodiments, a kit includes one or more containers, each with one or more of various materials (such as reagents, optionally in concentrated form, and/or devices) desirable from a commercial and user standpoint for use of a crystalline form described herein. Nonlimiting examples of such materials include, but are not limited to, buffers, diluents, filters, needles, syringes, carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded, or etched onto the container itself, a label is associated with a container when it is present within a receptacle or cater that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms containing a crystalline form provided herein. The pack may contain metal or plastic foil, such as a blister pack. In some embodiments, the pack or dispenser device is accompanied by instructions for administration. Optionally, the pack or dispenser is accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a crystalline form provided herein formulated in a compatible pharmaceutical carrier are prepared, placed in an appropriate container, and labeled for treatment of an indicated condition.

Combination Therapy

The crystalline forms and pharmaceutical compositions of the disclosure may be used in combination with one or more therapeutic agents which act by the same mechanism or by a different mechanism to treat a disease. The one or more agents may be administered sequentially or simultaneously, in separate compositions or in the same composition. Useful classes of agents for combination therapy include, but are not limited to, compounds used to treat cardiac, kidney, pulmonary, liver, skin, immunological and oncological conditions.

In practicing any of the subject methods, an ALK5 inhibitor (e.g., a crystalline form disclosed herein, such as polymorph Form I, polymorph Form II or polymorph Form III) and a second therapeutic agent can be administered sequentially, wherein the two agents are introduced into a subject at two different time points. The two time points can be separated by more than 2 hours, 1 or more days, 1 or more weeks, 1 or more months, or according to any intermittent regimen schedule disclosed herein.

In some embodiments, the ALK5 inhibitor and the second therapeutic agent are administered simultaneously. The two agents may form part of the same composition, or the two agents may be provided in one or more unit doses.

In some embodiments, the ALK5 inhibitor or the second therapeutic agent are administered parenterally, orally, inhalatively, intraperitoneally, intravenously, intraarterially, transdermally, intramuscularly, liposomally, via local delivery by catheter or stent, subcutaneously, intraadiposally, or intrathecally. As used herein, a therapeutically effective amount of a combination of an ALK5 inhibitor and a second therapeutic agent refers to a combination of an ALK5 inhibitor and a second therapeutic agent, wherein the combination is sufficient to affect the intended application, including but not limited to, disease treatment, as defined herein. Also contemplated in the subject methods is the use of a sub-therapeutic amount of an ALK5 inhibitor and a second therapeutic agent in combination for treating an intended disease condition. The individual components of the combination, though present in sub-therapeutic amounts, synergistically yield an efficacious effect and/or reduced a side effect in an intended application.

The amount of the ALK5 inhibitor and the second therapeutic agent administered may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

Measuring an immune response and/or the inhibition of biological effects of ALK5 can comprise performing an assay on a biological sample, such as a sample from a subject. Any of a variety of samples may be selected, depending on the assay. Examples of samples include, but are not limited to blood samples (e.g. blood plasma or serum), exhaled breath condensate samples, bronchoalveolar lavage fluid, sputum samples, urine samples, and tissue samples.

A subject being treated with an ALK5 inhibitor and a second therapeutic agent may be monitored to determine the effectiveness of treatment, and the treatment regimen may be adjusted based on the subject's physiological response to treatment. For example, if inhibition of a biological effect of ALK5 inhibition is above or below a threshold, the dosing amount or frequency may be decreased or increased, respectively. Alternatively, the treatment regimen may be adjusted with respect to an immune response. The methods can further comprise continuing the therapy if the therapy is determined to be efficacious. The methods can comprise maintaining, tapering, reducing, or stopping the administered amount of a compound or compounds in the therapy if the therapy is determined to be efficacious. The methods can comprise increasing the administered amount of a compound or compounds in the therapy if it is determined not to be efficacious. Alternatively, the methods can comprise stopping therapy if it is determined not to be efficacious. In some embodiments, treatment with an ALK5 inhibitor and a second therapeutic agent is discontinued if inhibition of the biological effect is above or below a threshold, such as in a lack of response or an adverse reaction. The biological effect may be a change in any of a variety of physiological indicators.

Specific agents that may be used in combination with the crystalline forms disclosed herein include, but are not limited to, OFEV® (nintedanib) and Esbriet® (pirfenidone). In some embodiments, a crystalline form disclosed herein is administered in combination with pirfenidone, optionally wherein the pirfenidone is administered by inhalation. In some embodiments, the present disclosure provides a method of treating fibrosis, such as idiopathic pulmonary fibrosis, in a subject, comprising administering to the subject an ALK5 inhibitor, such as polymorph Form I, polymorph Form II or polymorph Form III, and nintedanib or pirfenidone. In some embodiments, the present disclosure provides a method of treating cancer, such as lung cancer, in a subject, comprising administering to the subject an ALK5 inhibitor, such as polymorph Form I, polymorph Form II or polymorph Form III, and nintedanib or pirfenidone.

In some embodiments, the present disclosure provides a method for treating a proliferative disorder (e.g., lung cancer) in a subject in need thereof, comprising administering to said subject an ALK5 inhibitor and an immunotherapeutic agent. TGF-β has been shown to regulate lymphocyte differentiation, suppress T cell proliferation and to enhance tumor growth. Moreover, TGF-β has been shown to prevent optimal activation of the immune system in immunotherapy-resistant patients (see Loffek, S. J. Oncolo. 2018, 1-9; incorporated herein by reference in its entirety). Not wishing to be bound by any particular theory, the present inventors expect that inhibition of ALK5 may enhance the efficacy of a particular immunotherapy. As such, treatment with an immunotherapeutic agent, such as durvalumab or pembrolizumab, and an ALK5 inhibitor, such as a crystalline form of the present disclosure, is expected to improve the clinical outcome of a subject with cancer, such as a subject with non-small cell lung cancer. The combination is expected to produce a synergistic effect. A synergistic combination is also expected for a triple combination of radiation therapy, immunotherapy, and ALK5 inhibition. In addition, the ALK5 inhibitor, even when administered locally (e.g., to the lung by inhalation), may stimulate both local and systemic immune responses, allowing for the treatment of primary or metastatic tumors in tissues beyond the site of the local delivery. For example, an inhaled ALK5 inhibitor may be administered in combination with an immunotherapeutic agent to treat melanoma, renal cell carcinoma, colon cancer, or breast cancer.

In some embodiments, the ALK5 inhibitor and the immunotherapeutic agent are administered sequentially or simultaneously. In some embodiments, the ALK5 inhibitor and the immunotherapeutic agent are more effective in treating the proliferative disorder than either agent alone. In some embodiments, the ALK5 inhibitor and the immunotherapeutic agent yield a synergistic effect in treating the proliferative disorder. The synergistic effect may be a therapeutic effect that is greater than either agent used alone in comparable amounts under comparable conditions. The synergistic effect may be a therapeutic effect that is greater than results expected by adding the effects of each agent alone. In some embodiments, the proliferative disorder is a cancer condition. In some embodiments, the cancer condition is lung cancer, such as non-small cell lung cancer.

The term “immunotherapeutic agent” refers to any agent that induces, enhances, suppresses or otherwise modifies an immune response. This includes the administration of an active agent to, or any type of intervention or process performed on, the subject, with the objective of modifying an immune response. An immunotherapeutic agent may, for example, increase or enhance the effectiveness or potency of an existing immune response in a subject, for example, by stimulating mechanisms that enhance the endogenous host immune response or overcoming mechanisms that suppress the endogenous host immune response.

“Immune response” refers to the action of a cell of the immune system including, for example, B lymphocytes, T lymphocytes, natural killer (NK) cells, macrophages, eosinophils, mast cells, myeloid-derived suppressor cells, dendritic cells and neutrophils and soluble macromolecules produced by any of these cells or the liver (including antibodies, cytokines and complement), that results in selective targeting, binding to, damage to, destruction of, and/or elimination of invading pathogens, cells or tissues infected with pathogens, cancerous or other abnormal cells, or, in cases of autoimmunity or pathological inflammation, normal human cells or tissues, from the body of a subject.

In one embodiment, an immunotherapeutic agent may comprise a PD-1 inhibitor. In another embodiment, an immunotherapeutic agent may comprise a CTLA-4 inhibitor. In still another embodiment, an immunotherapeutic agent may comprise a B7 inhibitor.

Exemplary PD-1 inhibitors: A PD-1 inhibitor suitable for use in the subject methods can be selected from a variety of types of molecules. For example, the PD-1 inhibitor can be a biological or chemical compound, such as an organic or inorganic molecule, peptide, peptide mimetic, antibody or an antigen-binding fragment of an antibody. Some exemplary classes of agents suitable for use in the subject methods are detailed in the sections below. A PD-1 inhibitor for use in the present disclosure can be any PD-1 inhibitor that is known in the art, and can include any entity that, upon administration to a patient, results in inhibition of the PD-1 pathway in the patient. A PD-1 inhibitor can inhibit PD-1 by any biochemical mechanism, including disruption of any one or more of PD-1/PD-L1, PD1/PD-L2 and PD-L1/CD80 interactions.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-1 inhibitor is a molecule that inhibits the binding of PD-L1 to its binding partners. In a specific aspect, PD-L1 binding partners are PD1 and/or CD80. In another embodiment, the PD-1 inhibitor is a molecule that inhibits the binding of PD-L2 to its binding partners. In a specific aspect, a PD-L2 binding partner is PD1. The inhibitor may be an antibody, an antigen binding fragment thereof, an immunoadhesin, a fusion protein or oligopeptide.

In some embodiments, the PD-1 inhibitor is an anti-PD-1 antibody. In some further embodiments, the anti-PD-1 antibody is capable of inhibiting binding between PD-1 and PD-L1. In another embodiment, the anti-PD-1 antibody is capable of inhibiting binding between PD-1 and PD-L2. In some embodiments, the PD-1 inhibitor is an anti-PD-L1 antibody. In some embodiments, the anti-PD-L1 antibody is capable of inhibiting binding between PD-L1 and PD-1 and/or between PD-L1 and CD80. In some embodiments, the PD-1 inhibitor is an anti-PD-L2 antibody. In some further embodiments, the anti-PD-L2 antibody is capable of inhibiting binding between PD-1 and PD-L2. In yet another embodiment, the PD-1 inhibitor is nivolumab or pembrolizumab. In some embodiments, the PD-1 inhibitor is selected from atezolizumab, avelumab, nivolumab, pembrolizumab, durvalumab and BGB-A317.

Inhibition of the PD-1 pathway can enhance the immune response to cancerous cells in a patient. The interaction between PD-1 and PD-L1 impairs T cell response as manifested by a decrease in tumor-infiltrating lymphocytes (TILs) and a decrease in T-cell receptor mediated proliferation, resulting in T cell anergy, exhaustion or apoptosis, and immune evasion by the cancerous cells. This immune suppression can be reversed by inhibiting the local interaction between PD-L1 and PD-1 using a PD-1 inhibitor, including, for example, an anti-PD-1 and/or an anti-PD-L1 Ab. A PD-1 inhibitor may improve or restore antitumor T-cell functions.

Anti-PD-1 antibodies suitable for use in the disclosure can be generated using methods well known in the art. Exemplary PD-1 inhibitors include, but are not limited to: nivolumab (BMS936558), pembrolizumab (MK-3475), pidilizumab (CT-011), AMP-224, AMP-514, BMS-936559, RG7446 (MPDL3280A), MDX-1106 (Medarex Inc.), MSB0010718C, MED14736, and HenGrui mAB005 (WO 15/085847). Further PD-1 antibodies and other PD-1 inhibitors include those described in WO 04/056875, WO 06/121168, WO 07/005874, WO 08/156712, WO 09/014708, WO 09/114335, WO 09/101611, WO 10/036959, WO 10/089411, WO 10/027827, WO 10/077634, WO 11/066342, WO 12/145493, WO 13/019906, WO 13/181452, WO 14/022758, WO 14/100079, WO 14/206107, WO 15/036394, WO 15/085847, WO 15/112900, WO 15/112805, WO 15/112800, WO 15/109124, WO 15/061668, WO 15/048520, WO 15/044900, WO 15/036927, WO 15/035606; U. S. Pub. No. 2015/0071910; and U.S. Pat. Nos. 7,488,802; 7,521,051; 7,595,048; 7,722, 868; 7,794,710; 8,008,449; 8,354,509; 8,383,796; 8,652,465; and 8,735,553; all of which are incorporated herein by reference. Some anti-PD-1 antibodies are commercially available, for example from ABCAM (AB137132), BIOLEGEND (EH12.2H7, RMP 1-14) and AFFYMETRIX EBIOSCIENCE (J105, J116, M1H4).

Exemplary CTLA-4 inhibitors: A CTLA-4 inhibitor suitable for use in the subject methods can be selected from a variety of types of molecules. For example, the CTLA-4 inhibitor can be a biological or chemical compound, such as an organic or inorganic molecule, peptide, peptide mimetic, antibody or an antigen-binding fragment of an antibody. Some exemplary classes of agents suitable for use in the subject methods are detailed in the sections below. A CTLA-4 inhibitor for use in the present disclosure can be any CTLA-4 inhibitor that is known in the art, and can include any entity that, upon administration to a patient, results in inhibition of the CTLA-4 pathway in the patient. A CTLA-4 inhibitor can inhibit CTLA-4 by any biochemical mechanism, including disruption of either one or both of CTLA-4/CD80 and CTLA-4/CD86 interactions.

In some embodiments, the CTLA-4 inhibitor is a molecule that inhibits the binding of CTLA-4 to its ligand binding partners. In a specific aspect, the CTLA-4 ligand binding partners are CD80 and/or CD86. In another embodiment, a CTLA-4 inhibitor is a molecule that inhibits the binding of CD80 to its binding partners. In a specific aspect, a CD80 binding partner is CTLA-4. In another embodiment, the CTLA-4 inhibitor is a molecule that inhibits the binding of CD86 to its binding partners. In a specific aspect, a CD86 binding partner is CTLA-4. The inhibitor may be an antibody, an antigen binding fragment thereof, an immunoadhesin, a fusion protein or oligopeptide.

In some embodiments, the CTLA-4 inhibitor is an anti-CTLA-4 antibody. In some further embodiments, the anti-CTLA-4 antibody is capable of inhibiting binding between CTLA-4 and CD80. In another embodiment, the anti-CTLA-4 antibody is capable of inhibiting binding between CTLA-4 and CD86. In some embodiments, the CTLA-4 inhibitor is an anti-CD80 antibody. In some embodiments, the anti-CD80 antibody is capable of inhibiting binding between CTLA-4 and CD80. In some embodiments, the CTLA-4 inhibitor is an anti-CD86 antibody. In some further embodiments, the anti-CD86 antibody is capable of inhibiting binding between CTLA-4 and CD86. In yet another embodiment, the CTLA-4 inhibitor is tremelimumab or ipilimumab.

Inhibition of the CTLA-4 pathway can enhance the immune response to cancerous cells in a patient. The interaction between CTLA-4 and one of its natural ligands, CD80 and CD86, delivers a negative regulatory signal to T cells. This immune suppression can be reversed by inhibiting the local interaction between CD80 or CD86 and CTLA-4 using a CTLA-4 inhibitor, including, for example, an anti-CTLA-4 Ab, anti-CD80 Ab or an antiCD86 Ab. A CTLA-4 inhibitor may improve or restore antitumor T-cell functions.

Anti-CTLA-4 antibodies suitable for use in the disclosure can be generated using methods well known in the art. Exemplary CTLA-4 inhibitors include but are not limited to tremelimumab and ipilimumab (also known as 10D1 or MDX-010). Further CTLA-4 antibodies and other CTLA-4 inhibitors include those described in WO 98/042752, WO 00/037504, WO 01/014424 and WO 04/035607; U. S. Pub. Nos. 2002/0039581, 2002/086014 and 2005/0201994; U.S. Pat. Nos. 5,811,097; 5,855,887; 5,977,318; 6,051,227; 6,207, 156; 6,682,736; 6,984,720; 7, 109,003; 7, 132,281; 7,605,238; 8, 143,379; 8,318,916; 8,435,516; 8,784,815; and 8,883,984; EP Pat. No. 1212422; Hurwitz et al., PNAS 1998, 95(17): 10067-10071; Camacho et al., J Clin Oncology 2004, 22(145): abstract no. 2505 (antibody CP675206); and Mokyr, et al., Cancer Research 1998, 58:5301-5304; each of which is incorporated herein by reference.

Also provided herein is a pharmaceutical composition comprising a crystalline form of the disclosure and one or more other therapeutic agents. The therapeutic agent may be selected from the classes of agents specified above and from the lists of specific agents described above. In some embodiments, the pharmaceutical composition is suitable for delivery to the lungs. In some embodiments, the pharmaceutical composition is suitable for inhaled or nebulized administration. In some embodiments, the pharmaceutical composition is a dry powder or a liquid composition.

Further, in a method aspect, the disclosure provides a method of treating a disease or disorder in a mammal comprising administering to the mammal a crystalline form of the disclosure and one or more other therapeutic agents.

When used in combination therapy, the agents may be formulated in a single pharmaceutical composition, or the agents may be provided in separate compositions that are administered simultaneously or at separate times, by the same or by different routes of administration. Such compositions can be packaged separately or may be packaged together as a kit. The two or more therapeutic agents in the kit may be administered by the same route of administration or by different routes of administration.

### EXAMPLES

The following examples are given for the purpose of illustrating various embodiments of the disclosure and are not meant to limit the present disclosure in any fashion. The present examples, along with the methods and compositions described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

The following abbreviations have the following meanings unless otherwise indicated and any other abbreviations used herein and not defined have their standard, generally accepted meaning:
AcOH=acetic acid
Atm=atmosphere
Boc₂O=di-tert-butyl dicarbonate
BSA=bovine serum albumin, Fraction V
d=day(s)
DCM=dichloromethane or methylene chloride
DMF=N,N-dimethylformamide
DMSO=dimethyl sulfoxide
DTT=dithiothreitol
EDTA=ethylenediaminetetraacetic acid
EGTA=ethylene glycol-bis(O-aminoethyl ether)-N,N,N′,N′-tetraacetic acid
EtOAc or EA=ethyl acetate
g=gram(s)
h=hour(s)
HEPES=4-(2-hyrdroxyethyl)-1-piperazine ethanesulfonic acid
KHMDS=potassium bis(trimethylsilyl)amide
MeOH=methanol
min=minute(s)
Pd₂(dba)₃=tris(dibenzylideneacetone)dipalladium(O)
PE=petroleum ether
RT, rt, or r.t.=room temperature
SEMCl=2-(trimethylsilyl)ethoxymethyl chloride
SiO₂=silicon dioxide or silica
TFA=trifluoroacetic acid
THF=tetrahydrofuran
Tris-HCl=tris(hydroxymethyl)aminomethane hydrochloride
Tween-20=polyoxyethylene sorbitan monolaurate
Xantphos=4,5-bis(diphenylphosphino)-9,9-dimethylxanthene

Unless noted otherwise, all materials, such as reagents, starting materials and solvents, were purchased from commercial suppliers, such as Sigma-Aldrich, Fluka Riedel-de Haën, and the like, and were used without further purification.

Reactions were run under nitrogen atmosphere, unless noted otherwise. The progress of reactions was monitored by thin layer chromatography (TLC), analytical high performance liquid chromatography (anal. HPLC), and mass spectrometry, the details of which are given in specific examples.

Reactions were worked up as described specifically in each preparation; commonly, reaction mixtures were purified by extraction and other purification methods such as temperature- and solvent-dependent crystallization, and precipitation. In addition, reaction mixtures were routinely purified by preparative HPLC, typically using Microsorb C18 and Microsorb BDS column packings and conventional eluents. Progress of reactions was typically measured by liquid chromatography mass spectrometry (LCMS). Characterization of isomers was typically done by Nuclear Overhauser effect spectroscopy (NOE). Characterization of reaction products was routinely carried out by mass spectrometry and/or ¹H-NMR spectroscopy. For NMR measurement, samples were dissolved in deuterated solvent (CD₃OD, CDCl₃, or DMSO-d₆), and ¹H-NMR spectra were acquired with a Varian Gemini 2000 instrument (400 MHz) under standard observation conditions. Mass spectrometric identification of compounds was typically conducted using an electrospray ionization method (ESMS) with an Applied Biosystems (Foster City, Calif.) model API 150 EX instrument or an Agilent (Palo Alto, Calif.) model 1200 LC/MSD instrument.

### Example 1: Synthesis of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, trifluoroacetic acid (I trifluoroacetate)

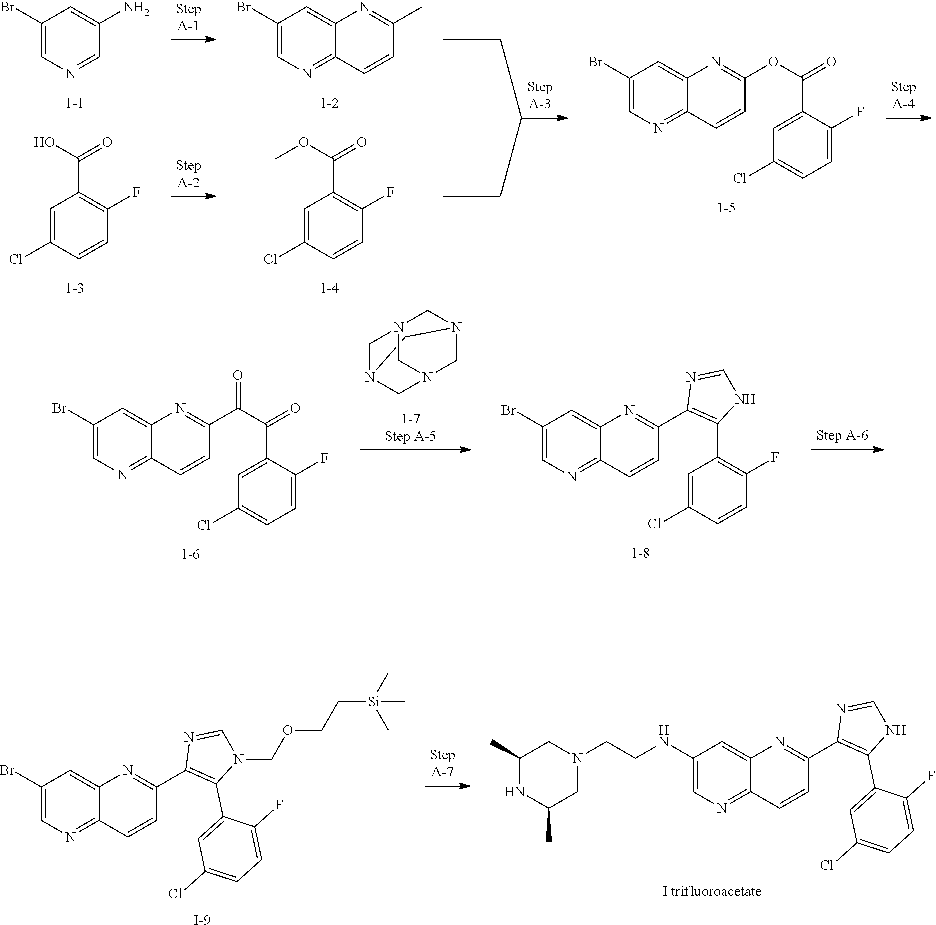

Step A-1: Synthesis of 7-bromo-2-methyl-1,5-napthyridine (1-2). (E)-but-2-enal (30.66 g, 437 mmol) in toluene (90 mL) was added dropwise to 5-bromopyridin-3-amine (18.0 g, 104.0 mmol) in HCl (1.8 L, 6 M) at 100° C. and the mixture was stirred for 1 h at 100° C. A further amount of (E)-but-2-enal (30.66 g, 437 mmol) in toluene (90 mL) was added in one portion and the mixture was stirred at 100° C. for another 4 h. The solvent was removed in vacuum to dryness and the pH of the residue was adjusted to pH 8.0 with NaHCO₃ solid. This procedure was repeated four times and the crude products were combined and purified by column chromatography (PE:EA=100:1 to 5:1) to yield 1-2 as a yellow solid (71 g, 95% purity, 15.3% yield). [M+H]⁺ calcd for C₉H₈BrN₂ 222.99, found 222.9. ¹H NMR (400 MHz, CDCl₃) δ 8.89 (d, J=1.6 Hz, 1H), 8.46 (d, J=1.6 Hz, 1H), 8.23 (d, J=8.8 Hz, 1H), 7.50 (d, J=8.4 Hz, 1H), 2.76 (s, 3H).

Step A-2: Synthesis of methyl 5-chloro-2-fluorobenzoatone (1-4). To a mixture of 5-chloro-2-fluorobenzoic acid 1-3 (80 g, 458.3 mmol) in MeOH (800 mL), SOCl₂ (162 g, 1374.9 mmol) was added dropwise. The reaction was stirred at 15° C. for 16 h before being concentrated in vacuo. The concentrate was then diluted with H₂O (500 mL) and adjusted to pH 8 by adding saturated aqueous NaHCO₃. The mixture was extracted with EtOAc (3×300 mL). The combined organic layers were washed with brine (2×300 mL), dried with Na₂SO₄, concentrated in vacuum and purified by column chromatography (PE:EA=100:1 to 20:1) to yield compound 1-4 as a colorless oil (80 g, 95% purity, 93% yield). [M+H]⁺ calcd for C₈H₇ClFO₂ 189.01, found 189.0. ¹H NMR (400 MHz, Chloroform-d) δ 7.95 (dd, J=6.0, 2.8 Hz, 1H), 7.52-7.46 (m, 1H), 7.14 (t, J=5.6 Hz, 1H), 3.96 (s, 3H).

Step A-3: Synthesis of 2-(7-bromo-1,5-naphthyridin-2-yl)-1-(5-chloro-2-fluorophenyl)ethan-1-one (1-5). KHMDS (81 mL, 81.08 mmol, 1M) was added dropwise to a mixture of compound 1-2 (9 g, 40.54 mmol) and compound 1-4 (23 g, 121.62 mmol) in THF (250 mL) at −78° C. The mixture was then stirred at −78° C. for 1 h before being warmed to 15° C. and stirred for another 30 min. The mixture was quenched with H₂O (400 mL) to form a yellow solid precipitate. This procedure was repeated once and the combined precipitate was filtered. The resultant filter cake was washed with H₂O (50 mL) and further titrated with PE/EA=5:1 (180 mL) to yield intermediate 1-5 as a yellow solid (27 g, 95% purity, 88% yield). [M+H]⁺ calcd for C₁₆H₉BrClFN₂O 378.96, found 378.9.

Step A-4: Synthesis of 1-(7-bromo-1,5-naphthyridin-2-yl)-2-(5-chloro-2-fluorophenyl)ethane-1,2-dione (1-6). A solution of 1-5 (10.9 g, 28.7 mmol) and SeO₂ (15.9 g, 144 mmol) in dioxane (200 mL) was stirred at 100° C. for 3.5 h. The reaction mixture was filtered through a pad of celite. The filtrate was concentrated in vacuum to give 1-6 as a yellow solid (11 g, 97% yield, 82% purity). [M+H]⁺ calcd for C₁₆H₇BrClFN₂O₂ 392.95, found 393.0.

Step A-5: Synthesis of 7-bromo-2-(5-(5-chloro-2-fluorophenyl)-1H-imidazol-4-yl)-1,5-naphthyridine (1-8). A solution of 1-6 (11.0 g, 22.92 mmol), 1,3,5,7-tetraazaadamantane 1-7 (9.6 g, 68.76 mmol) and NH₄OAc (10.6 g, 137.5 mmol) in AcOH (100 mL) was heated to 95° C. After 30 min., additional AcOH (100 mL) was added, and the reaction mixture was stirred for 2 h. The reaction mixture was concentrated in vacuo and basified with sat. aq. NaHCO₃ (300 mL) to pH 9. The mixture was extracted with EA (3×400 mL). The combined organic phase was washed with brine (2×400 mL), dried over Na₂SO₄, filtered, and concentrated under vacuum to give a residue. The residue was purified by column (EA:MeOH=1:0 to 10:1) to yield 1-8 as a yellow solid (6.0 g, 55% yield, 95% purity). [M+H]⁺ calcd for C₁₇H₉BrClFN₄ 402.98, found 403.1.

Step A-6: Synthesis of 7-bromo-2-(5-(5-chloro-2-fluorophenyl)-1-((2-(trimethylsilyl)ethoxy)methyl)-1H-imidazol-4-yl)-1,5-naphthyridine (1-9). To a solution of 1-8 (6.0 g, 14.86 mmol) in DMF (120 mL) was added NaH (773 mg, 19.32 mmol) at 0° C. The reaction mixture was stirred at 0° C. for 1 h. SEMCl (3.0 g, 17.83 mmol) was then added to the mixture. The reaction mixture was stirred at 20° C. for 2 h, then diluted with H₂O (200 mL) and extracted with EA (3×300 mL). The organic layer was washed with brine (2×300 mL), dried over Na₂SO₄, filtered, and concentrated in vacuo. The residue was purified by column (PE:EA=10:0 to 3:1) to afford 1-9 as a yellow solid (3.3 g, 42% yield, 98% purity. [M+H]⁺ calcd C₂₃H₂₃BrClFN₄OSi 533.06, found 533.2. ¹H NMR (400 MHz, DMSO-d₆) δ 8.93 (d, J=2.0 Hz, 1H), 8.49-8.38 (m, 2H), 8.25 (s, 1H), 7.87-7.81 (m, 1H), 7.72-7.62 (m, 2H), 7.42 (t, J=8.9 Hz, 1H), 5.32 (s, 2H), 3.42 (t, J=8.2 Hz, 2H), 0.78 (t, J=8.2 Hz, 2H), −0.08 (s, 9H) and ¹H NMR (400 MHz, DMSO-d₆) δ 9.08 (d, J=2.2 Hz, 1H), 8.77 (dd, J=2.2, 0.9 Hz, 1H), 8.39 (dd, J=8.8, 1.0 Hz, 1H), 8.26 (s, 1H), 7.71 (dd, J=6.3, 2.8 Hz, 1H), 7.59 (d, J=8.8 Hz, 1H), 7.48 (ddd, J=8.8, 4.2, 2.8 Hz, 1H), 7.20 (dd, J=9.9, 8.8 Hz, 1H), 5.87 (s, 2H), 3.36-3.29 (m, 2H), 0.60 (dd, J=8.6, 7.4 Hz, 2H), −0.29 (s, 9H).

Step A-7: Synthesis of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, trifluoroacetic acid (I trifluoroacetate). To a solution of 1-9 (33.30 mg, 0.062 mmol) and tert-butyl (2S,6R)-4-(2-((tert-butoxycarbonyl)amino)ethyl)-2,6-dimethylpiperazine-1-carboxylate (27.0 mg, 0.075 mmol) in PhCH₃ (0.249 mL) was added Pd₂dba₃ (1.43 mg, 1.56 μmol), XantPhos (0.90 mg, 1.56 μmol) and sodium tert-butoxide (18.0 mg, 0.187 mmol). The resulting mixture was degassed with N₂ and heated to 100° C. for 2 h. The reaction was filtered through a plug of celite and concentrated in vacuo. The resulting residue was dissolved in TFA (0.50 mL) and heated to 50° C. for 1 h. The crude product was concentrated in vacuo and purified by preparative HPLC chromatography using a gradient (2 to 60%) of acetonitrile in water with 0.05% trifluoroacetic acid to yield the title TFA salt (33.4 mg). [M+H]⁺ calcd for C₂₅H₂₈ClFN₇ 480.2073 found 480.2067. ¹H NMR (601 MHz, Methanol-d₄) δ 9.29 (s, 1H), 8.88 (d, J=2.7 Hz, 1H), 8.39 (d, J=8.8 Hz, 1H), 8.02 (d, J=2.7 Hz, 1H), 7.80 (dd, J=6.0, 2.7 Hz, 1H), 7.72 (ddd, J=9.0, 4.4, 2.7 Hz, 1H), 7.55 (dd, J=8.9, 0.9 Hz, 1H), 7.42 (t, J=9.1 Hz, 1H), 4.02-3.93 (comp m, 6H), 3.67 (t, J=6.1 Hz, 2H), 3.39 (t, J=13.3 Hz, 2H), 1.46 (d, J=6.5 Hz, 6H).

### Example 2: Synthesis of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine (I)

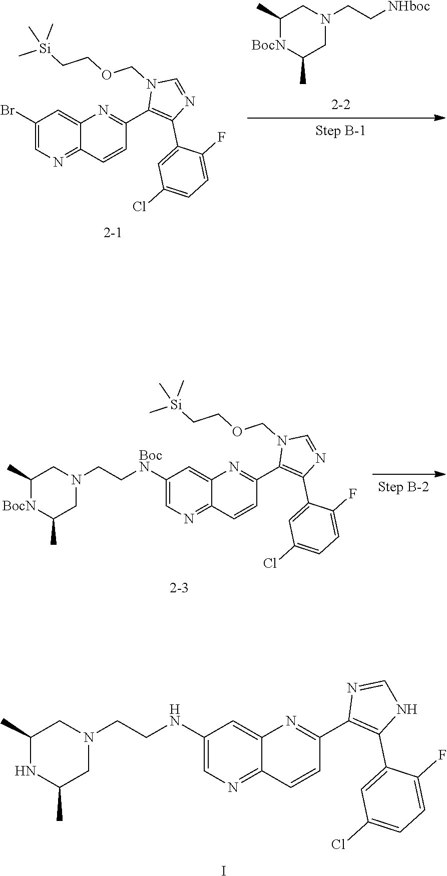

Step B-1: Synthesis of tert-butyl (2S,6R)-4-(2-((tert-butoxycarbonyl)(6-(4-(5-chloro-2-fluorophenyl)-1-((2-(trimethylsilyl)ethoxy)methyl)-1H-imidazol-5-yl)-1,5-naphthyridin-3-yl)amino)ethyl)-2,6-dimethylpiperazine-1-carboxylate (2-3). To a solution of 2-1 (10.00 g, 18.73 mmol), 2-2 (8.04 g, 22.48 mmol) and XantPhos (0.434 g, 0.749 mmol) in toluene (50 mL) was added t-BuONa (5.40 g, 56.2 mmol). The resulting mixture was transferred to a mixture of Pd₂dba₃ (0.686 g, 0.749 mmol) in toluene (15 mL) with rinsing (35 mL toluene). The resulting mixture was degassed with N₂ and heated to 100° C. for 12 h. The reaction was filtered through a plug of celite, rinsed with 1.5 volumes of toluene and the filtrate concentrated to afford a crude oil. The crude product was dissolved in DCM and purified by preparative silica gel chromatography using a gradient (20 to 50%) of EtOAc in hexanes to afford compound 2-3 as an oil (7.61 g, 50.1% yield).

Step B-2: Synthesis of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine (I). To a solution of 2-3 (23.0 g, 28.4 mmol) in toluene (100 mL) was added 5M aq. HCl (56.8 mL, 284 mmol). The resulting mixture was heated to 80° C. and stirred for 5 hours, then cooled to room temperature and water (50 mL) added. The toluene layer was split off, then discarded. The aqueous phase was extracted with EtOAc (100 mL) and the EtOAc layer was split off and discarded. MeTHF (100 mL) was added to the aqueous phase and the resulting mixture was adjusted to pH 12 with 5M aq. NaOH. The aqueous phase was extracted with MeTHF (2×) and the combined organic layers were dried over Na₂SO₄ and concentrated to reduce the volume to 70 mL. Trifluorotoluene (200 mL) was added and the total volume was distilled to 115 mL. Fresh trifluorotoluene (115 mL) was added and the resulting mixture stirred overnight to afford a fine slurry. Filtration and drying afforded the title compound (10.2 g, 73.5% yield, 98.2% purity). [M+H]⁺ calcd for C₂₅H₂₇ClFN₇ 480.20 found 480.2.

### Example 3: Synthesis of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, trihydrochloride (I.3HCl)

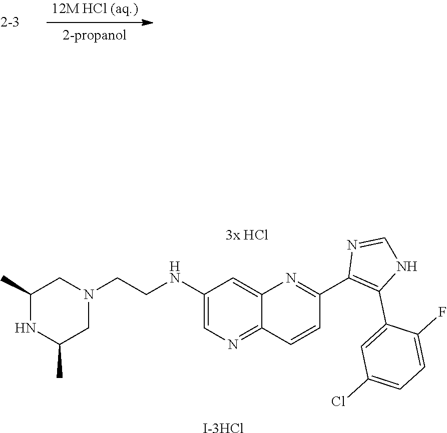 [SMILES: C[C@@H]1CN(CCNc2cnc3ccc(-c4nc[nH]c4-c4cc(Cl)ccc4F)nc3c2)C[C@H](C)N1 ; "I-3HCl"]

A solution of compound 2-3 (11.0 g, 13.57 mmol) in toluene (89 mL) was added to 12M HCl aq. (30.6 mL, 373 mmol) over 35 minutes under high agitation at 20° C. After 1 hour, agitation was stopped, and the toluene layer was split off and discarded. 2-Propanol (45.9 mL) was then charged to the aqueous solution over 30 minutes at 20° C. Seeds of I.3HCl were charged to the solution and the mixture was allowed to stir 16 hours, after which a thick slurry had developed. More 2-propanol (107 mL) was charged to the slurry over 3 hours. After an additional 24 hour hold at 20° C., the product was filtered and rinsed with 2-propanol (24 mL). The cake was dried for 20 hours under vacuum at 45° C., rendering I.3HCl (5.65 g, 69% yield, 98.2% purity).

### Example 4: Preparation of polymorph Form I of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric Acid (I fumarate)

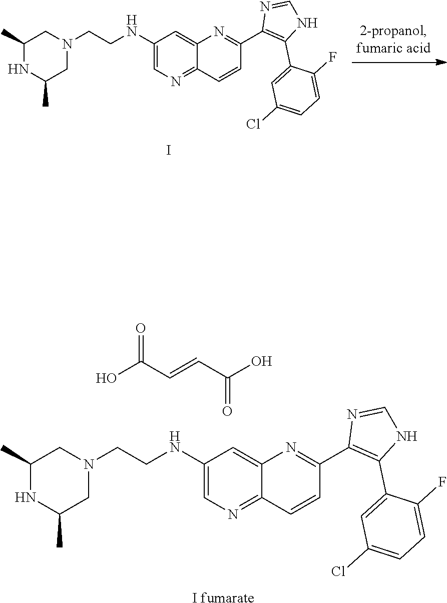

To a suspension of I (2.0 g, 4.17 mmol) in 2-propanol (20 mL) was added fumaric acid (0.484 g, 4.17 mmol). The slurry was heated to an internal temperature of 80° C. and held for 2 hours. Water (2 mL) was then added, and the slurry was held at 80° C. for an additional hour, then cooled to 50° C. and held for 3 days. Following this hold, the slurry was cooled to ambient temperature, filtered, and the wet cake was dried under nitrogen. The resulting fumarate salt was isolated as polymorph Form I in 81% yield (2 g) with an HPLC purity of 98.5%.

### Example 5: Preparation of polymorph Form II of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric Acid (I fumarate)

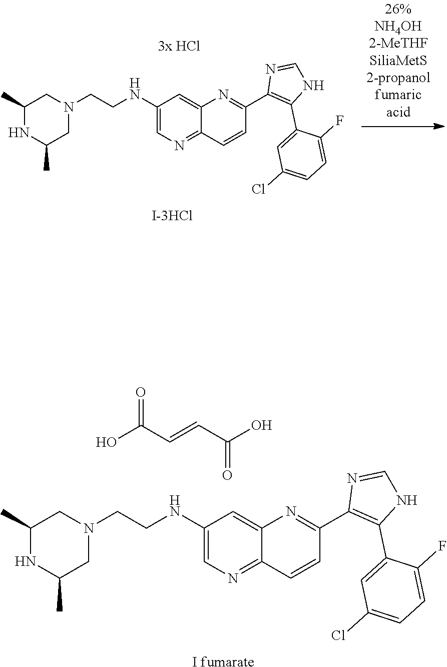

To a solution of compound I.3HC1 (4.0 g, 6.79 mmol) in water (40.0 mL) under nitrogen blanket was added a mixture of 2-methyltetrahydrofuran (40.0 mL) and 26% NH₄OH aq. (8.0 mL, 53.4 mmol) under agitation over 30 min. at 20° C. The mixture was then heated to 40° C. Agitation was stopped and the mixture was allowed to settle. The bottom layer (aqueous) was split off to waste. To the organic layer was charged additional 2-methyltetrahydrofuran (12.0 mL), and the mixture was vacuum distilled to a volume of 36 mL. To this organic solution was charged 0.8 g of Silicycle siliametS thiol (1.41 mmol/g, 40-63 μm). After a 2 hour stir at 20° C., the Silicycle siliametS thiol was removed via filtration and rinsed forward with 2-methyltetrahydrofuran (4.0 mL). The organic filtrate was then vacuum distilled to 16 mL. This solution was then diluted with 2-propanol (48.0 mL) and vacuum distilled to 16 mL, resulting in a slurry. In a separate vessel, fumaric acid (0.867 g, 7.47 mmol) was dissolved in a mixture of 2-propanol (64.0 mL) and water (4.0 mL) at 25° C. The fumaric acid solution was charged to the slurry of I free base over 20 min. The slurry was then heated to 80° C., resulting in complete dissolution of solids. While holding the internal temperature between 60° C. and 85° C., the solution was vacuum distilled to 40 mL. The resulting slurry was then held at 80° C. for 1 h, then the internal temperature was ramped to 25° C. over 3 hours. After an overnight hold at 25° C., the slurry was filtered and rinsed forward with 2-propanol (16.0 mL). The wet cake of I fumarate was dried in the oven at 40° C. overnight under vacuum and with a nitrogen bleed, resulting in a 98.2% yield (3.25 g) of polymorph Form II of I fumarate with an HPLC purity of 98.2%. ¹H NMR (600 MHz, DMSO-d₆) δ 8.50 (d, J=2.7 Hz, 1H), 8.11 (d, J=8.7 Hz, 1H), 8.00 (s, 1H), 7.72 (dd, J=2.7, 6.4 Hz, 1H), 7.48 (m, 2H), 7.22 (t, J=9.1 Hz, 1H), 7.13 (d, J=2.6 Hz, 1H), 6.73 (s, 2H), 3.42 (m, 4H), 3.20 (dd, J=2.9, 12.8 Hz, 2H), 2.82 (t, J=6.2, 2H), 2.16 (t, J=11.8 Hz, 2H), 1.34 (d, J=6.6 Hz, 6H). ¹³C NMR (150 MHz, DMSO-d₆) δ 170.0, 158.5, 151.3, 145.9, 145.3, 143.8, 136.7, 135.8, 134.8, 134.4, 132.4, 131.2, 129.9, 128.9, 127.7, 123.0, 117.7, 117.1, 108.4, 56.0, 55.1, 51.7, 39.6, 15.1. IR: 3400 (N—H), 2450, 2359 (aliphatic C—H), 1607, 1450, 1354 (heteroaromatic ring skeleton), 1491 (CH₃ δ asymmetric stretch), 1213 (C—F) cm⁻¹.

### Example 6: Alternative preparation of polymorph Form II of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric Acid (I fumarate)

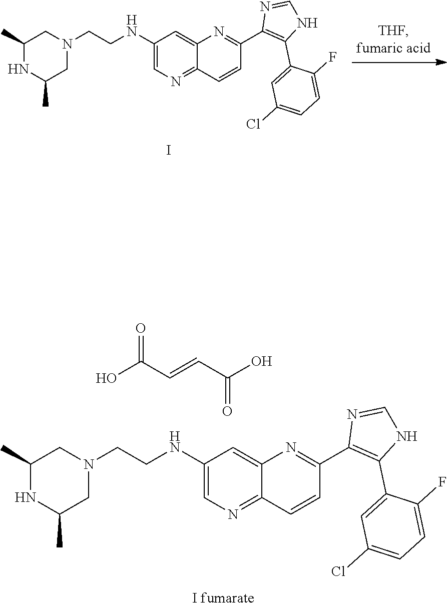

I (32.9 mg) and fumaric acid (10.5 mg) were suspended in THF (0.5 mL). The resulting suspension was stirred at room temperature for 1 day, then filtered, washed with THF (2 mL) and dried under ambient conditions for a few hours to provide polymorph Form II of I fumarate.

### Example 7: Preparation of polymorph Form III of 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine (I freebase)

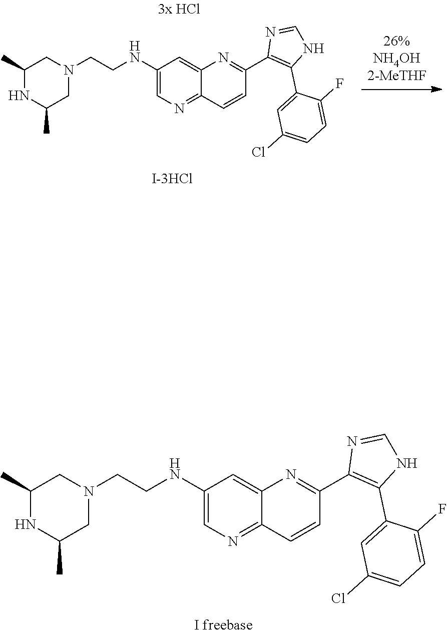 [SMILES: C[C@@H]1CN(CCNc2cnc3ccc(-c4nc[nH]c4-c4cc(Cl)ccc4F)nc3c2)C[C@H](C)N1 ; "I-3HCl" ; C[C@@H]1CN(CCNc2cnc3ccc(-c4nc[nH]c4-c4cc(Cl)ccc4F)nc3c2)C[C@H](C)N1 ; "I freebase"]

To a solution of compound I.3HC1 (10.0 g, 16.97 mmol) in water (200 mL) under nitrogen blanket was added a mixture of 2-methyltetrahydrofuran (200 mL) and 26% NH₄OH aq. (7.5 mL, 59.4 mmol) under agitation over 5 min. The mixture was then heated to 40° C. for 10 min. under nitrogen. Agitation was stopped and the mixture was allowed to settle. The bottom layer (aqueous) was split off to waste. The organic layer was vacuum distilled to a volume of approximately 70 mL. To the organic layer was charged degassed isopropanol (200 mL) and the resulting mixture was vacuum distilled to a volume of 30 mL. Additional isopropanol (20 mL) was added and the resulting mixture was degassed 3 times at 40° C. before adding seed crystals and stirring overnight at 40° C. The resulting slurry was cooled to room temperature and stirred under nitrogen for 3 days. The slurry was filtered and dried under nitrogen to afford crystalline I freebase (5.5 g, 67% yield, 99% purity).

### Example 8: X-Ray Powder Diffraction Analysis

The X-ray powder diffraction patterns depicted in FIG. 1, FIG. 5 and FIG. 9 were obtained with a Bruker D8-Advance X-ray diffractometer equipped with a Lynxeye 1D detector using Cu-Kα radiation (λ=1.54051 Å) with output voltage of 45 kV and current of 40 mA. The instrument was operated in Bragg-Brentano geometry with incident, divergence and scattering slits set to maximize the intensity at the sample. For measurement, a small amount of powder (5-25 mg) was gently pressed onto a sample holder to form a smooth surface and subjected X-ray exposure. The samples were scanned in 0-20 mode from 2° to 35° in 2θ with a step size of 0.02° and a scan speed of 2 seconds per step for a total of 1 hour scan time. The data acquisition was controlled by Bruker DiffracSuite measurement software and analyzed by Jade software (version 7.7). Representative XRPD patterns of polymorph Form I and polymorph Form II of a fumarate salt of the compound of Formula I and polymorph Form III of the compound of Formula I (freebase) are depicted in FIG. 1, FIG. 5 and FIG. 9, respectively. Observed XRPD 2° peak positions and d-spacings for polymorph Form I and polymorph Form II of a fumarate salt of the compound of Formula I and polymorph Form III of the compound of Formula I (freebase) are shown in Table 1, Table 3 and Table 5, respectively.

### Example 9: Thermal Analysis

Differential scanning calorimetry (DSC) measurements were performed using a TA Instruments Model Discovery DSC instrument. Data were collected with TRIOS software and analyzed using TA Instruments Universal Analysis software. A sample of the crystalline form of the compound of Formula I was weighed into an aluminum pan covered with a TZero hermetic lid. The sample was heated using a linear heating ramp of 10° C./min from 40° C. to 280° C. Representative DSC thermograms of polymorph Form I and polymorph Form II of a fumarate salt of the compound of Formula I and polymorph Form III of the compound of Formula I (freebase) are shown in FIG. 2, FIG. 6 and FIG. 10, respectively.

Thermogravimetric analysis (TGA) measurements were performed using a TA Instruments Model Discovery TGA module equipped with high resolution capability. Data were collected using TA Instruments TRIOS software and analyzed using TA Instruments Universal Analysis software. A weighed sample was placed onto a platinum pan and scanned with a heating rate of 10° C. per minute from ambient temperature to 300° C. The balance and furnace chambers were purged with nitrogen flow during use. Representative TGA traces of polymorph Form I and polymorph Form II of a fumarate salt of the compound of Formula I and polymorph Form III of the compound of Formula I (freebase) are shown in FIG. 3, FIG. 7 and FIG. 11, respectively.

### Example 10: Dynamic Moisture Sorption Analysis

Dynamic moisture sorption (DMS) analysis was performed on weighed samples of the crystalline forms of the compound of Formula I using a VTI atmospheric microbalance, SGA-100 system (VTI Corp., Hialeah, Fla. 33016). Following an initial drying step (approximately 0% RH) of 2 hours, two cycles of sorption and desorption were completed isothermally at 25° C. at a scan rate of 5% RH/step over a humidity range of 5 to 90% RH. Representative DMS traces of polymorph Form I and polymorph Form II of a fumarate salt of the compound of Formula I and polymorph Form III of the compound of Formula I (freebase) are shown in FIG. 4, FIG. 8 and FIG. 12, respectively.

### Example 11: Microcrystal Electron Diffraction

A continuous carbon grid was pressed against a sample of polymorph Form I of a fumarate salt of the compound of Formula I. The grid was gently tapped to remove excess sample and clipped at room temperature. The grid was dried in a vacuum oven set to 40° C. for 15 minutes, then stored in liquid nitrogen.

Electron microscopy was performed using a Thermo Fisher Scientific Glacios Cryo Transmission Electron Microscope (Cryo-TEM) operated at 200 kV, equipped with a Ceta-D detector and operated at cryogenic temperature (below −170° C.). Diffraction datasets were collected under parallel illumination conditions with a very low dose. A 20 μm condenser aperture was used during data collection, which resulted in approximately a 0.6 μm diameter beam on the specimen. Automated data collection was carried out using Leginon software. Leginon recorded the diffraction tilt series through the TEM User Interface with the camera set to record continuously in rolling shutter mode with 2×2 binning. The acquisition was synchronized with a slow tilting function.

Datasets were indexed, refined, integrated and scaled with the program DIALS (Diffraction Integration for Advanced Light Sources). Xia2 was used to convert file types and XPREP was used to analyze possible space groups. Initial phasing was carried out by dual space phasing in SHELXD. Refinement was carried out in SHELXL in Olex2 and validation was carried out in PLATON (CheckCIF). Hydrogens were modeled with interatomic distances based on values derived from neutron scattering. Hydrogens were also modeled as “riding”, based on idealized geometry and not allowed to freely refine against the experimental density. Unit cell parameters and space group details are provided in Table 2.

### Example 12: Single Crystal X-Ray Diffraction

A crystal of polymorph Form II of a fumarate salt of the compound of Formula I was mounted on a glass fiber. Data were collected on a Rigaku Atlas CCD diffractometer equipped with an Oxford Cryosystems Cobra cooling device, using Cu-Kα radiation. The crystal structure was solved and refined using Bruker AXS SHELXTL software. Hydrogen atoms attached to carbon atoms were placed geometrically and allowed to refine with a riding isotropic displacement parameter. Hydrogen atoms attached to heteroatoms were located in a difference Fourier map and were allowed to refine freely with an isotropic displacement parameter. Unit cell parameters, along with crystal system and space group details, are provided in Table 4.

### Example 13: Stability Study

Samples of the crystalline forms disclosed herein, such as polymorph Form I and polymorph Form II of a fumarate salt of the compound of Formula I and polymorph Form III of the compound of Formula I (freebase), are stored at 25° C. and 60% relative humidity (RH) or at accelerated conditions of 40° C. and 75% RH, then analyzed by HPLC. Relative peak areas of the compound of Formula I and detected impurities are recorded.

### Example 14: Biochemical ALK5 (TGF-βR1) Assay to Measure pKi

Apparent pKi values for the compound of Formula I were determined using a recombinant human ALK5 (TGF-βR1) protein (Product No. PR9075A or equivalent, Life Technologies) and a commercially-available kinase assay (LANCE® (lanthanide chelate excite) Ultra ULight™ kinase assay, Product Nos. TRF0130-M and TRF02108-M, Perkin Elmer) as described below.

The assays were performed in a 384-well plate (24 columns×16 wells/rows). An Echo®550 Liquid Handler (Labcyte) was used to prepare various intermediate concentrations of the compound of Formula I in 100% DMSO. From the intermediate concentrations, a range of concentrations (from 10 μM to 25 μM corresponding to volumes up to 105 nL) were prepared and ejected into a final assay plate to be used to create individual dose response curves. To a separate column within the assay plate, 105 nL of DMSO in each well was used to establish a maximum assay signal. Additionally, 105 nL of 100 μM SD-208, a selective TGF-βR1 inhibitor (Catalog #S7624, Selleck Chemicals), was used in another column of wells to establish a minimal assay signal.

With a multidrop dispenser, 8 μL of enzyme mixture (1.25× final) was added to each well. The enzyme mixture consisted of 250 μM ALK5 enzyme and 62.5 nM peptide substrate (LANCE® (lanthanide chelate excite) Ultra ULight™-DNA Topoisomerase 2-alpha (Thr1342)) prepared in assay buffer (50 mM HEPES, 10 mM MgCl₂, 1 mM EGTA, 0.01% Tween-20, pH 7.5 at room temperature) with 2 mM DTT added prior to use. The plate was then sealed with an adhesive seal and allowed to equilibrate for 60 minutes at room temperature.

Next, 2 μL of 125 μM ATP (5× final, 125 μM ATP prepared in assay buffer with 2 mM DTT) was added to the incubated mixtures, covered with a MicroClime® Environmental Lid (Product No. LLS-0310, Labcyte) and immediately transferred to 37° C. The reactions were allowed to proceed at 37° C. for 60 minutes before terminating with the addition of 10 μL of detection antibody (LANCE® (lanthanide chelate excite) Ultra Europium-anti-phospo-DNA Topoisomerase 2-alpha (Thr1342)) in detection mixture (12 mM EDTA, 4 nM detection antibody prepared in detection buffer (50 mM Tris-HCl, 150 mM NaCl, 0.5% BSA (Fraction V), pH 7.0)) at room temperature. The plate was then read on a Perkin Elmer EnVision Plate Reader using europium specific reader settings with excitation and emission wavelengths set to 320 or 340 nm and 665 nm, respectively. These data were used to calculate percent enzyme inhibition values based on DMSO and SD-208 background controls.

For dose-response analyses, percent inhibition versus compound concentrations were plotted, and pIC₅₀ values were determined from a 4-parameter robust fit model with GraphPad Prism V5 Software (GraphPad Software, Inc., La Jolla, Calif.). This model obtains pIC₅₀ values by fitting the sigmoidal dose-response (variable slope) equation to the data. Results were expressed as pIC₅₀ (negative logarithm of IC₅₀) and subsequently converted to pKᵢ (negative logarithm of dissociate constant, Kᵢ) using the Cheng-Prusoff equation. The higher the value of pKᵢ (lower value of Kᵢ), the greater the inhibition of ALK5 activity. The compound of Formula I exhibited a pKᵢ value between 9.5 and 10.4 when tested in the biochemical ALK5 assay.

### Example 15: Cellular ALK5 Potency Assay to Measure pIC₅₀, Inhibition of TGF-β Stimulated pSMAD3 Formation in BEAS-2B Cells

The potency of the compound of Formula I for inhibition of TGF-β-stimulated SMAD3 phosphorylation was measured in BEAS-2B cells, a human lung epithelial cell line. TGF-β signals through activin receptor-like kinase 5 (ALK5) immediately prior to SMAD3 phosphorylation. As the AlphaLISA SureFire Ultra kit (Perkin Elmer) quantitatively measures pSMAD3 levels in lysate, the assay demonstrates the ALK5 cellular potency of a test compound.

BEAS-2B cells were grown using 50% DMEM (Life Technologies) and 50% F-12 (Life Technologies) media, supplemented with 10% Fetal Bovine Serum (ATCC), 25 mM HEPES (Life Technologies), and 1× Pen-Strep (Life Technologies). Cells were cultured in a humidified incubator set at 37° C., 5% CO₂, and trypsonized using 0.25% Trypsin with 0.5% polyvinylpyrrolidone (PVP).

For the assay, BEAS-2B cells were seeded at 7,500 cells/well (25 μL/well) in a 384-well plate and cultured overnight. Before dosing, growth media was aspirated and the wells were rinsed with HBSS Buffer (HBSS with Calcium and Magnesium, Life Technologies) supplemented with 25 mM HEPES (Life Technologies) and 1% Bovine Serum Albumin (Roche). Compounds were serially diluted in DMSO, then further diluted with supplemented HBSS Buffer (50 μL/well) to create a compound plate 3× of the final assay concentration, at 0.3% DMSO. The diluted compounds were then added to the cells (8 μL/well) and incubated at 37° C., 5% CO₂ for 1 hour. After the compound incubation, TGF-β (R&D Systems) reconstituted in supplemented HBSS Buffer was added to the cells (12 μL/well, final concentration 10 ng/mL) and incubated for a further 30 minutes, after which the cells were immediately lysed with AlphaLISA lysis buffer (PerkinElmer). AlphaLISA Acceptor and Detector beads (PerkinElmer) were added 2 hours apart, then incubated overnight to be read the next day. The potency of the compound was determined through analysis of dose-dependent quantified changes in pSMAD3 signal from baseline (non-compound treated TGF-β stimulated cells). Data are expressed as pIC₅₀ (negative decadic logarithm IC₅₀) values. The compound of Formula I exhibited a pIC₅₀ value between 6.8 and 7.6 when tested in BEAS-2B cells.

### Example 16: Cytotoxicity Measured by Premature Chromosome Condensation [15] (pCC₁₅)

The impact of the compound of Formula I on cellular adenosine triphosphate (ATP) levels was measured in Beas2B cells, a human lung epithelial cell line. Levels of ATP are correlated with the viability of cells and are often measured to determine the potential cytotoxicity of compounds. CellTiter-Glo, which lyses the cells and produces a luminescent signal proportional to the amount of ATP present, was used to determine the effect of test compound on cell viability.

Beas2B cells were grown in 50% DMEM (Life Technologies) and 50% F-12 (Life Technologies) media, supplemented with 10% Fetal Bovine Serum (ATCC), 25 mM HEPES (Life Technologies), and 1× Pen-Strep (Life Technologies). Cells were cultured in a humidified incubator set at 37° C., 5% CO₂, and trypsinized using 0.25% Trypsin with 0.5% polyvinylpyrrolidone (PVP).

For the assay, Beas2B cells were seeded at 500 cells/well (25 μL/well) in a 384-well plate and cultured overnight. Compounds were serially diluted in DMSO, then further diluted with growth media (40 μL/well) to create a compound plate 6× of the final assay concentration, at 0.6% DMSO. The diluted compounds were then added to the cells (5 μL/well) and incubated at 37° C., 5% CO₂ for 48 hours. After the compound incubation, CellTiter-Glo (Promega) was added directly to the cells (30 μL/mL). The assay plate was sealed and shaken at 700 rpm for 15 minutes in a darkened environment, then centrifuged for 2 minutes at 1500 rpm to settle the lysate at the bottom of the well. The effect of the compound on cell viability was determined through analysis of dose-dependent quantified changes in ATP from baseline (non-compound treated cells) and wells treated with 60 μM AT9283, a well-characterized cytotoxic compound. Data are expressed as pCC₁₅ (negative decadic logarithm CC₁₅) values. The compound of Formula I exhibited a pCC₁₅ value between 5.1 and 5.7 when tested in Beas2B cells.

### Example 17: In Vitro Human Liver Microsome Intrinsic Clearance (HLM Clᵢₙₜ)

Liver microsomes were used for in vitro determination of hepatic clearance of the compound of Formula I. A microsomal incubation cofactor solution was prepared with 100 mM potassium phosphate buffered to pH 7.4 (BD Biosciences, Woburn, Mass.) supplemented with 2 mM NADPH (Sigma-Aldrich, St. Louis, Mo.). 10 mM DMSO stocks of test compound were diluted and spiked into the cofactor solution to yield a 0.2 μM concentration (0.02% v/v DMSO). Aliquots of frozen human liver microsomes (Bioreclamation IVT, Baltimore Md.) were thawed and diluted into 100 mM potassium phosphate buffer to yield microsomal protein concentrations of 0.2 mg/mL. Cofactor/drug and microsomal solutions were pre-warmed separately for 4 minutes in a water bath held at 37° C. Incubations (n=1) were started by the combination of equal volumes of cofactor/drug solution with microsomal solution. The final concentration of test compound was 0.1 μM with a final protein concentration of 0.1 mg/mL and final NADPH concentration of 1 mM. Samples were collected at times 0, 3, 8, 15, 30, and 45 minutes to monitor the disappearance of test compound. At each time point, 50 μL of incubation sample was removed and spiked into 25 μL of water plus 3% formic acid plus Internal Standard for reaction termination. Samples were then injected onto an AB Sciex API 4000 triple quadrupole mass spectrometer for quantitation by LC-MS/MS. Mobile Phase A consisted of HPLC grade water with 0.2% formic acid and Mobile Phase B consisted of HPLC grade acetonitrile with 0.2% formic acid with all samples run through a Thermo HyPURITY C18 50×2.1 mm column (Waltham, Mass.). HLM Clᵢₙₜ data was reported in units of μL/min/mg. See Riley, R. J., et al., Drug Metab. Dispos., 2005, September, 33(9), pp. 1304-1311. The compound of Formula I exhibited an HLM Clᵢₙₜ of greater than 250 μL/min/mg.

### Example 18: Lung PK/PD

In-Life Portion

C57bl/6n mice were acclimated for at least 3 days before use. On the day of the experiment, animals were grouped into sample sizes of 5 (n=10 for the TGF-β stimulated group). The compound of Formula I (formulated in 3% glycerol in PBS; pH=4) was pre-treated via oral aspiration (OA; animals are forced to aspirate solution into the lungs by covering their nose). All oral aspirations were performed using a 50 μL dosing volume and accompanied by the appropriate vehicle control groups. Following compound OA treatment, the animals were returned to their home cages and monitored. Compound pre-treatment occurred 4 hours prior to harvest for screening and dose-response studies; duration studies had variable compound pre-treatment times. One hour prior to harvest, animals were challenged via oral aspiration a second time with PBS vehicle or recombinant human TGF-β1 protein (0.01 μg per animal dissolved in 1 part 4 mM HCl and 2 parts 3% glycerol in PBS). Five minutes prior to harvest, animals were deeply anesthetized under isoflurane and euthanized via cervical dislocation. Bronchoalveolar lavage fluid (BALF), plasma and left lung lobes were collected during harvest.

Sample Collection and Processing

Blood plasma was collected via open cardiac puncture. After whole blood collection, the samples were placed in EDTA-coated tubes to prevent coagulation. Blood samples were spun at 15300×g's for 4 minutes at 4° C. to separate the plasma. Plasma was immediately isolated, frozen and submitted for bioanalytical (BA) analysis.

In order to collect BALF, the lungs were flushed via the trachea with 0.7 mL of PBS 3 times. The BALF, which consists almost entirely of tissue-derived macrophages, was immediately centrifuged at 700×g's for 15 minutes. After centrifugation, the supernatant was removed, the BALF was re-suspended in 1× cell lysis buffer, and immediately frozen. Prior to BA submission, the BALF was dethawed and sonicated for 30 minutes on cold water to lyse open the cells

Left lung lobes were harvested immediately after BALF collection. Lung samples were homogenized in 500 μL of 1× cell lysis buffer. After homogenization, the samples were split: half of the sample was immediately placed on a rotisserie for 10 minutes while the other half was immediately frozen for BA analysis. The samples placed on the rotisserie were then centrifuged at 10,000×g's for 10 minutes in order to separate the protein in the supernatant from pelleted debris. Following collection of the supernatant, a total protein quantification assay (Bradford) was performed to normalize the concentrations of all samples. Using the Hamilton star liquid handling system, each sample was diluted in 1× cell lysis buffer to 2 mg/mL of protein. Samples were stored at −80° C. or immediately processed using the Meso-scale Discovery system.

Phospho-SMAD3 (pSMAD3) and Total-SMAD3 (tSMAD3) Quantification Using Meso-Scale Discovery

Meso-scale Discovery (MSD) is an electrochemical protein quantification assay that requires specialized microplates with carbon electrodes attached to the bottom. These carbon electrodes allow for greater attachment of biological reagent to microplates, thus allowing for a more sensitive read-out when compared to a traditional ELISA. Similar to a standard sandwich ELISA, MSD requires use of a coating antibody that binds the target protein(s) within the sample. After sample incubation, a primary antibody is used to bind the epitope of interest. Following addition of the primary antibody, a secondary-antibody with a SULFO-TAG detection is used to allow for quantification of the epitope of interest. Lastly, the microplate is read via an electric pulse that causes the SULFO-TAG to emit light, which serves as the final read-out of the assay.

The coating antibody (SMAD3, clone=5G-11) was incubated overnight in the specialized MSD microplates at 4° C. The next day, the microplates were blocked in 3% BSA (bovine serum albumin) for 70 minutes to prevent non-specific protein binding to the bottom of the microplate. After a wash step, 50 μg of lung samples were loaded into the MSD-plate and incubated for 2 hours at room temperature. The plates were washed again to remove unbound sample; either phospo-SMAD3 (pSMAD3; clone=EP568Y) or total-SMAD3 (tSMAD3) primary antibody were incubated for 1 hour. Following a wash step, the anti-rabbit SULFO-tag detection antibody was incubated for 50 minutes. After a final wash step, MSD-read buffer was added to each sample. pSMAD3 and tSMAD3 quantification was performed using an MSD-specific plate reader (Sector S 600).

Data Analysis

Samples were immediately analyzed using an outlier analysis (Grubbs test, a=0.05). After outlier removal, the raw pSMAD3 were divided by the tSMAD3 luminescent readings. In screening and dose-response studies, the pSMAD3/tSMAD3 ratio was normalized to the TGF-β induction group (set to 100%) in order to minimize the variability between stimulation. First, the 3% glycerol/PBS group was compared with the 3% glycerol/TGF-β with a student's t-test (cut-off: p=0.05) to ensure a pSMAD3 window was present. A one-way ANOVA (fisher's uncorrected LSD) was used to compare all drug treated groups with the 3% glycerol/TGF-β group to determine if statistically significant differences are observed. Percent pSMAD3 inhibition was calculated using the vehicle pSMAD3 as a baseline value and displayed as the final readout. Dose-response curves were fitted with a 4-parameter non-linear regression algorithm; the minimum response was set to 0% pSMAD3 inhibition and the maximum response set to 100% pSMAD3 inhibition. Compound potencies were obtained from the regression and reported as ID50s.

PK Study

Plasma, lung and macrophage drug concentrations were quantified. Total macrophage concentration was normalized to the total macrophage cell volume over the total drug recovered in the BALF. The alveolar macrophage volume used in the calculation was based on a publication by Krombach et al. (Environmental Health Perspectives, September 1997, Vol. 105, Supplement 5, pp. 1261-1263) which estimated the rat alveolar macrophage volume to be approximately 1200 μm³ or 1.2e⁻⁹ mL. The assumption was made that the mouse alveolar macrophage volume is similar to that of the rat. Normalized total macrophage concentration recovered=(total drug recovered from BALF)/(total cell counts*1.2e⁻⁹ mL). The compound of Formula I exhibited a (lung AUC₀₋₄):(plasma AUC₀₋₄) ratio of greater than 75.

### Example 19: Cardiac PK/PD

In-Life Portion

C57bl/6n mice were acclimated for at least 3 days before use. On the day of the experiment, animals were grouped into sample sizes of 5-10. Test compounds were pre-treated via oral aspiration (OA; animals are forced to aspirate solution into the lungs by covering their nose). All oral aspirations were performed using a 50 μL dosing volume and accompanied by a vehicle control group (3% glycerol in PBS, pH=4). Following compound OA treatment, the animals were returned to their home cages and monitored. Compound pre-treatment occurred either 2 or 4 hours prior to harvest. One hour prior to harvest, animals were challenged via tail-vein intravenous injection with PBS vehicle or recombinant human TGF-β1 protein (1 μg per animal dissolved in 1 part 4 mM HCl and 2 parts 3% glycerol in PBS). Five minutes prior to harvest, animals were deeply anesthetized under isoflurane and euthanized via cervical dislocation. Plasma, left lung lobes and whole hearts were collected during harvest.

Sample Collection and Processing

Blood plasma was harvested as described above in the Lung PK/PD experiment. Whole hearts were processed in the same manners as left lung lobes in the Lung PK/PD experiment. Left lung lobes were homogenized in 500 μL of water and submitted for BA Analysis.

Phospho-SMAD3 (pSMAD3) and Total-SMAD3 (tSMAD3) Quantification Using Meso-Scale Discovery

Heart samples were processed using MSD in the same manner as the left lung lobes above. Data analysis was performed in the same manners as the lung PK/PD experiment. Plasma, lung and heart drug concentrations were quantified. There was minimal target engagement systemically following treatment with the compound of Formula I, as measured by SMAD3 phosphorylation inhibition.

### Example 20: Efficacy Study in Syngeneic Cancer Model

The compound of Formula I is expected to suppress tumor growth in syngeneic cancer models when administered alone or in combination with an immunotherapeutic agent. Six- to 8-week old BALB/c mice are used for in vivo efficacy studies in accordance with IACUC guidelines. Commercially available 4T1 cells (0.5-2.0×10⁴ cells/mouse) are implanted subcutaneously into the right flanks of BALB/c mice. When the tumor reaches a palpable size of approximately 8-10 mm in diameter, the primary tumors are surgically removed, and the mice are randomly assigned to vehicle control or compound treatment groups. Alternatively, CT26 cells (0.5-2.0×10⁴ cells/mouse) are injected intravenously into BALB/c mice to generate the cancer model. Two days following the surgery, or 7 days following injection of CT26 cells, the mice are treated with either (1) vehicle control, (2) the compound of Formula I at an appropriate amount and frequency (formulated in 3% glycerol in PBS; pH=4) via oral aspiration or intranasally, (3) an immunotherapeutic agent (e.g., pembrolizumab or durvalumab) at an appropriate amount and frequency, or (4) the compound of Formula I and an immunotherapeutic agent, each at an appropriate amount and frequency.

Body weight is measured twice weekly. Following 2- to 4-weeks of treatment, the lung and liver of each animal is harvested, and the number of metastatic cells in each tissue sample determined using a clonogenic metastasis assay. Cells may be further subjected to one or more of FACS analysis, T-cell function assay, and RNA extraction. It is expected that the animal group treated with the compound of Formula I exhibits reduction in lung tumor burden. Activation of an immune response by the ALK5 inhibitor may stimulate both local and systemic antitumor T-cell activation, thus a reduction in liver tumor burden may also be observed. When administered in combination with an immunotherapeutic agent, the compound of Formula I, is expected to produce an increased reduction in lung tumor burden relative to the reduction in tumor burden observed in animals treated with either single agent alone. The compound of Formula I is expected to interact synergistically with an immunotherapeutic agent to suppress tumor growth and increase survival.

### Example 21: Prophylactic Study in Murine DSS-Induced Intestinal Fibrosis Model

The compound of Formula I is expected to slow, halt or reverse the progression of intestinal fibrosis in a murine colitis model. Six to 8-week old male C57BL/6J mice are tagged and weighed. The drinking water of the animals is treated with 2.5% dextran sulfate sodium (DSS) for 7 days to induce acute colitis, followed by 2 days of normal drinking water. Three, 3-week cycles of 2.5% DSS treatment (1 week of 2.5% DSS in water; 2 weeks of normal water) are then completed to induce intestinal fibrosis.

Starting on day one of DSS administration, mice are treated with either vehicle control or the compound of Formula I at an appropriate amount and frequency via oral gavage (e.g., once daily). The animals are sacrificed 9 weeks after the first DSS administration, then distal, mid and proximal sections of the colon harvested for histologic analysis, RNA extraction and cytokine measurement. The compound of Formula I is expected to decrease ALK5 activity in the colon and to slow or prevent intestinal fibrosis as evidenced by one or more of (1) reduction in the ratio of colon weight to colon length; (2) reduction in deposition of extracellular matrix as observed by histology; (3) reduction in expression of collagen 1 (Colla1) and connective tissue growth factor (Ctg) in colon tissue; and (4) reduction in production of TGF-β1 and IL6 in the colon, relative to vehicle-treated controls.

### Example 22: Efficacy Study in Murine DSS-Induced Intestinal Fibrosis Model

The compound of Formula I is expected to slow, halt or reverse the progression of intestinal fibrosis in a murine colitis model. Six to 8-week old male C57BL/6J mice are tagged and weighed. The drinking water of the animals is treated with 2.5% dextran sulfate sodium (DSS) for 7 days to induce acute colitis, followed by 2 days of normal drinking water. Three, 3-week cycles of 2.5% DSS treatment (1 week of 2.5% DSS in water; 2 weeks of normal water) are then completed to induce intestinal fibrosis.

Following the second of the 3 cycles of DSS administration, mice are treated with either vehicle control or the compound of Formula I at an appropriate amount and frequency via oral gavage (e.g., once daily). Animals are sacrificed at either 6, 9 or 12 weeks after the first DSS cycle, then distal, mid and proximal sections of the colon harvested for histologic analysis, RNA extraction and cytokine measurement. The compound of Formula I is expected to decrease ALK5 activity in the colon and to slow, halt or reverse intestinal fibrosis as evidenced by one or more of (1) reduction in the ratio of colon weight to colon length; (2) reduction in deposition of extracellular matrix as observed by histology; (3) reduction in expression of collagen 1 (Colla1) and connective tissue growth factor (Ctgf) in colon tissue; and (4) reduction in production of TGF-β1 and IL6 in the colon, relative to vehicle-treated controls.

### Example 23: Efficacy Study in Adoptive T-Cell Transfer Model of Colitis

The compound of Formula I is expected to slow, halt or reverse the progression of intestinal fibrosis in an adoptive T-cell transfer model of colitis. Six- to 8-week old female CB17 SCID mice are tagged and weighed, then administered CD4⁺ CD25⁻ CD62L⁺ naïve T cells isolated from the spleens of Balb/C mice (IP; 1×10⁶ cells) to induce colitis.

Once diarrhea and a 10% or greater decrease in body weight are observed (typically around week 2), mice are treated with either vehicle control or the compound of Formula I at an appropriate amount and frequency via oral gavage (e.g., once daily). Animals are sacrificed 45 days after induction of colitis, then distal, mid and proximal sections of the colon harvested for histologic analysis, RNA extraction and cytokine measurement. The compound of Formula I is expected to decrease ALK5 activity in the colon and to slow, halt or reverse intestinal fibrosis as evidenced by one or more of (1) reduction in the ratio of colon weight to colon length; (2) reduction in deposition of extracellular matrix as observed by histology; (3) reduction in expression of collagen 1 (Colla1) and connective tissue growth factor (Ctgf) in colon tissue; and (4) reduction in production of TGF-β1 and IL6 in the colon, relative to vehicle-treated controls.

### Example 24: Efficacy Study in Monocrotaline Model of Severe Pulmonary Hypertension

The compound of Formula I is expected to slow, halt or reverse the progression of pulmonary hypertension in a monocrotaline (MCT) model of severe pulmonary hypertension. Male Sprague-Dawley rats are tagged, weighed, and randomly divided into control and MCT-treated groups. The rats in the MCT-treated group are administered a single dose of MCT (60 mg/kg, s.c.), then treated with either (1) vehicle control; (2) sildenafil (30 mg/kg, p.o., b.i.d.); or (3) the compound of Formula I at an appropriate amount and frequency (formulated in 3% glycerol in PBS; pH=4) via oral aspiration.

Following 2-weeks of treatment, the animals are anesthetized with ketamine/xylazine for terminal monitoring of pulmonary and systemic arterial pressures along with heart rate. The lungs of each animal are then harvested for histologic analysis. The compound of Formula I is expected to decrease ALK5 activity in the lung and slow, halt or reverse the progression of pulmonary hypertension as evidenced by one or more of (1) reduction in systolic pulmonary arterial pressure; (2) reduction in right ventricular (RV) systolic pressure; (3) reduction in RV diastolic pressure; (4) increase in cardiac output; (5) reduction in RV hypertrophy; (6) reduction in pSmad2 or pSmad3 staining within vascular and/or alveolar cells; (7) reduction in medial thickness; (8) reduction in vascular smooth muscle cell proliferation; (9) reduction in vascular smooth muscle hypertrophy; and (10) reduction in expression of matrix metalloproteinase (MMP)-2 and/or MMP-9.

1-48. (canceled)

49. A fumarate salt of a compound of Formula I.

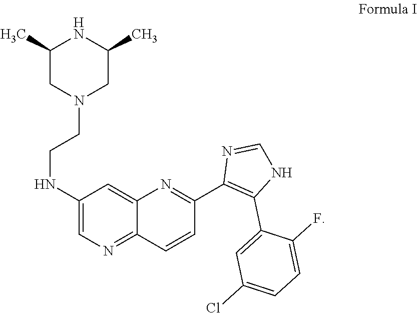 [SMILES: C[C@@H]1CN(CCNc2cnc3ccc(-c4nc[nH]c4-c4cc(Cl)ccc4F)nc3c2)C[C@H](C)N1]

50. A fumarate salt which is 6-(5-(5-chloro-2-fluorophenyl)-1H-imidazol-4-yl)-N-(2-((3S,5R)-3,5-dimethylpiperazin-1-yl)ethyl)-1,5-naphthyridin-3-amine, fumaric acid.

51. A pharmaceutical composition comprising the fumarate salt of claim 49 and a pharmaceutically acceptable carrier.

52. The pharmaceutical composition of claim 51, wherein the pharmaceutical composition is formulated for inhalation.

53. (canceled)

54. A method of treating an ALK5-mediated disease or condition in a subject, comprising administering to the subject a therapeutically effective amount of the fumarate salt of claim 49.

55. (canceled)

56. The method of claim 54, wherein the disease or condition is fibrosis.

57. (canceled)

58. (canceled)

59. The method of claim 56, wherein the fibrosis is selected from cardiac fibrosis, kidney fibrosis, pulmonary fibrosis, liver fibrosis, portal vein fibrosis, skin fibrosis, bladder fibrosis, intestinal fibrosis, peritoneal fibrosis, myelofibrosis, oral submucous fibrosis, and retinal fibrosis.

60. The method of claim 59, wherein the pulmonary fibrosis is selected from idiopathic pulmonary fibrosis (IPF), familial pulmonary fibrosis (FPF), interstitial lung fibrosis, fibrosis associated with asthma, fibrosis associated with chronic obstructive pulmonary disease (COPD), silica-induced fibrosis, asbestos-induced fibrosis and chemotherapy-induced lung fibrosis.

61. The method of claim 56, wherein the fibrosis is idiopathic pulmonary fibrosis (IPF).

62. (canceled)

63. The method of claim 56, wherein the fibrosis is intestinal fibrosis.

64. (canceled)

65. The method of claim 54, wherein the disease or condition is selected from breast cancer, colon cancer, prostate cancer, lung cancer, hepatocellular carcinoma, glioblastoma, melanoma, and pancreatic cancer.

66. The method of claim 65, wherein the lung cancer is non-small cell lung cancer.

67. The method of claim 54, comprising administering a second therapeutic agent.

68. The method of claim 67, wherein the second therapeutic agent is an immunotherapeutic agent.

69-71. (canceled)

72. The method of claim 54, wherein the fumarate salt is administered by inhalation.

73-87. (canceled)

88. A pharmaceutical composition comprising the fumarate salt of claim 50 and a pharmaceutically acceptable carrier.

89. A method of treating an ALK5-mediated disease or condition in a subject, comprising administering to the subject a therapeutically effective amount of the fumarate salt of claim 50.

90. The method of claim 89, wherein the disease or condition is idiopathic pulmonary fibrosis (IPF).

91. The method of claim 89, wherein the disease or condition is intestinal fibrosis.

92. The method of claim 89, wherein the disease or condition is non-small cell lung cancer.
